SEQUENCE LISTING (77 sequences)

SEQ ID NO: 1 - DNA, 2682 bp, Streptococcus pneumoniae TIGR4
atgcttaacagggagacacacatgaaaaaagtaagaaagatatttcagaaggcagttgcaggactgtgctgtatatctcagttgacagctttttcttcgatagttgctttagcagaaacgcctgaaaccagtccagcgataggaaaagtagtgattaaggagacaggcgaaggaggagcgcttctaggagatgccgtctttgagttgaaaaacaatacggatggcacaactgtttcgcaaaggacagaggcgcaaacaggagaagcgatattttcaaacataaaacctgggacatacaccttgacagaagcccaacctccagttggttataaaccctctactaaacaatggactgttgaagttgagaagaatggtcggacgactgtccaaggtgaacaggtagaaaatcgagaagaggctctatctgaccagtatccacaaacagggacttatccagatgttcaaacaccttatcagattattaaggtagatggttcggaaaaaaacggacagcacaaggcgttgaatccgaatccatatgaacgtgtgattccagaaggtacactttcaaagagaatttatcaagtgaataatttggatgataaccaatatggaatcgaattgacggttagtgggaaaacagtgtatgaacaaaaagataagtctgtgccgctggatgtcgttatcttgctcgataactcaaatagtatgagtaacattcgaaacaagaatgctcgacgtgcggaaagagctggtgaggcgacacgttctcttattgataaaattacatctgattcagaaaatagggtagcgcttgtgacttatgcttccactatctttgatgggaccgagtttacagtagaaaaaggggtagcagataaaaacggaaagcgattgaatgattctcttttttggaattatgatcagacgagttttacaaccaataccaaagattatagttatttaaagctgactaatgataagaatgacattgtagaattaaaaaataaggtacctaccgaggcagaagaccatgatggaaatagattgatgtaccaattcggtgccacttttactcagaaagctttgatgaaggcagatgagattttgacacaacaagcgagacaaaatagtcaaaaagtcattttccatattacggatggtgtcccaactatgtcgtatccgattaattttaatcatgctacgtttgctccatcatatcaaaatcaactaaatgcattttttagtaaatctcctaataaagatggaatactattaagtgattttattacgcaagcaactagtggagaacatacaattgtacgcggagatgggcaaagttaccagatgtttacagataagacagtttatgaaaaaggtgctcctgcagctttcccagttaaacctgaaaaatattctgaaatgaaggcggctggttatgcagttataggcgatccaattaatggtggatatatttggcttaattggagagagagtattctggcttatccgtttaattctaatactgctaaaattaccaatcatggtgaccctacaagatggtactataacgggaatattgctcctgatgggtatgatgtctttacggtaggtattggtattaacggagatcctggtacggatgaagcaacggctactagttttatgcaaagtatttctagtaaacctgaaaactataccaatgttactgacacgacaaaaatattggaacagttgaatcgttatttccacaccatcgtaactgaaaagaaatcaattgagaatggtacgattacagatccgatgggtgagttaattgatttgcaattgggcacagatggaagatttgatccagcagattacactttaactgcaaacgatggtagtcgcttggagaatggacaagctgtaggtggtccacaaaatgatggtggtttgttaaaaaatgcaaaagtgctctatgatacgactgagaaaaggattcgtgtaacaggtctgtaccttggaacggatgaaaaagttacgttgacctacaatgttcgtttgaatgatgagtttgtaagcaataaattttatgataccaatggtcgaacaaccttacatcctaaggaagtagaacagaacacagtgcgcgacttcccgattcctaagattcgtgatgtgcggaagtatccagaaatcacaatttcaaaagagaaaaaacttggtgacattgagtttattaaggtcaataaaaatgataaaaaaccactgagaggtgcggtctttagtcttcaaaaacaacatccggattatccagatatttatggagctattgatcaaaatggcacttatcaaaatgtgagaacaggtgaagatggtaagttgacctttaaaaatctgtcagatgggaaatatcgattatttgaaaattctgaaccagctggttataaacccgttcaaaataagcctatcgttgccttccaaatagtaaatggagaagtcagagatgtgacttcaatcgttccacaagatataccagcgggttacgagtttacgaatgataagcactatattaccaatgaacctattcctccaaagagagaatatcctcgaactggtggtatcggaatgttgccattctatctgataggttgcatgatgatgggaggagttctattatacacacggaaacatccgtaa

SEQ ID NO: 2 - protein, 893 aa, Streptococcus pneumoniae TIGR4
MLNRETHMKKVRKIFQKAVAGLCCISQLTAFSSIVALAETPETSPAIGKVVIKETGEGGALLGDAVFELKNNTDGTTVSQRTEAQTGEAIFSNIKPGTYTLTEAQPPVGYKPSTKQWTVEVEKNGRTTVQGEQVENREEALSDQYPQTGTYPDVQTPYQIIKVDGSEKNGQHKALNPNPYERVIPEGTLSKRIYQVNNLDDNQYGIELTVSGKTVYEQKDKSVPLDVVILLDNSNSMSNIRNKNARRAERAGEATRSLIDKITSDSENRVALVTYASTIFDGTEFTVEKGVADKNGKRLNDSLFWNYDQTSFTTNTKDYSYLKLTNDKNDIVELKNKVPTEAEDHDGNRLMYQFGATFTQKALMKADEILTQQARQNSQKVIFHITDGVPTMSYPINFNHATFAPSYQNQLNAFFSKSPNKDGILLSDFITQATSGEHTIVRGDGQSYQMFTDKTVYEKGAPAAFPVKPEKYSEMKAAGYAVIGDPINGGYIWLNWRESILAYPFNSNTAKITNHGDPTRWYYNGNIAPDGYDVFTVGIGINGDPGTDEATATSFMQSISSKPENYTNVTDTTKILEQLNRYFHTIVTEKKSIENGTITDPMGELIDLQLGTDGRFDPADYTLTANDGSRLENGQAVGGPQNDGGLLKNAKVLYDTTEKRIRVTGLYLGTDEKVTLTYNVRLNDEFVSNKFYDTNGRTTLHPKEVEQNTVRDFPIPKIRDVRKYPEITISKEKKLGDIEFIKVNKNDKKPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPAGYEFTNDKHYITNEPIPPKREYPRTGGIGMLPFYLIGCMMMGGVLLYTRKHP

SEQ ID NO: 3 - DNA, 1998 bp, Streptococcus pneumoniae TIGR4
atgaaatcaatcaacaaatttttaacaatgcttgctgccttattactgacagcgagtagcctgttttcagctgcaacagtttttgcggctgggacgacaacaacatctgttaccgttcataaactattggcaacagatggggatatggataaaattgcaaatgagttagaaacaggtaactatgctggtaataaagtgggtgttctacctgcaaatgcaaaagaaattgccggtgttatgttcgtttggacaaatactaataatgaaattattgatgaaaatggccaaactctaggagtgaatattgatccacaaacatttaaactctcaggggcaatgccggcaactgcaatgaaaaaattaacagaagctgaaggagctaaatttaacacggcaaatttaccagctgctaagtataaaatttatgaaattcacagtttatcaacttatgtcggtgaagatggagcaaccttaacaggttctaaagcagttccaattgaaattgaattaccattgaacgatgttgtggatgcgcatgtgtatccaaaaaatacagaagcaaagccaaaaattgataaagatttcaaaggtaaagcaaatccagatacaccacgtgtagataaagatacacctgtgaaccaccaagttggagatgttgtagagtacgaaattgttacaaaaattccagcacttgctaattatgcaacagcaaactggagcgatagaatgactgaaggtttggcattcaacaaaggtacagtgaaagtaactgttgatgatgttgcacttgaagcaggtgattatgctctaacagaagtagcaactggttttgatttgaaattaacagatgctggtttagctaaagtgaatgaccaaaacgctgaaaaaactgtgaaaatcacttattcggcaacattgaatgacaaagcaattgtagaagtaccagaatctaatgatgtaacatttaactatggtaataatccagatcacgggaatactccaaagccgaataagccaaatgaaaacggcgatttgacattgaccaagacatgggttgatgctacaggtgcaccaattccggctggagctgaagcaacgttcgatttggttaatgctcagactggtaaagttgtacaaactgtaactttgacaacagacaaaaatacagttactgttaacggattggataaaaatacagaatataaattcgttgaacgtagtataaaagggtattcagcagattatcaagaaatcactacagctggagaaattgctgtcaagaactggaaagacgaaaatccaaaaccacttgatccaacagagccaaaagttgttacatatggtaaaaagtttgtcaaagttaatgataaagataatcgtttagctggggcagaatttgtaattgcaaatgctgataatgctggtcaatatttagcacgtaaagcagataaagtgagtcaagaagagaagcagttggttgttacaacaaaggatgctttagatagagcagttgctgcttataacgctcttactgcacaacaacaaactcagcaagaaaaagagaaagttgacaaagctcaagctgcttataatgctgctgtgattgctgccaacaatgcatttgaatgggtggcagataaggacaatgaaaatgttgtgaaattagtttctgatgcacaaggtcgctttgaaattacaggccttcttgcaggtacatattacttagaagaaacaaaacagcctgctggttatgcattactaactagccgtcagaaatttgaagtcactgcaacttcttattcagcgactggacaaggcattgagtatactgctggttcaggtaaagatgacgctacaaaagtagtcaacaaaaaaatcactatcccacaaacgggtggtattggtacaattatctttgctgtagcgggggctgcgattatgggtattgcagtgtacgcatatgttaaaaacaacaaagatgaggatcaacttgcttaa

SEQ ID NO: 4 - protein, 665 aa, Streptococcus pneumoniae TIGR4
MKSINKFLTMLAALLLTASSLFSAATVFAAGTTTTSVTVHKLLATDGDMDKIANELETGNYAGNKVGVLPANAKEIAGVMFVWTNTNNEIIDENGQTLGVNIDPQTFKLSGAMPATAMKKLTEAEGAKFNTANLPAAKYKIYEIHSLSTYVGEDGATLTGSKAVPIEIELPLNDVVDAHVYPKNTEAKPKIDKDFKGKANPDTPRVDKDTPVNHQVGDVVEYEIVTKIPALANYATANWSDRMTEGLAFNKGTVKVTVDDVALEAGDYALTEVATGFDLKLTDAGLAKVNDQNAEKTVKITYSATLNDKAIVEVPESNDVTFNYGNNPDHGNTPKPNKPNENGDLTLTKTWVDATGAPIPAGAEATFDLVNAQTGKVVQTVTLTTDKNTVTVNGLDKNTEYKFVERSIKGYSADYQEITTAGEIAVKNWKDENPKPLDPTEPKVVTYGKKFVKVNDKDNRLAGAEFVIANADNAGQYLARKADKVSQEEKQLVVTTKDALDRAVAAYNALTAQQQTQQEKEKVDKAQAAYNAAVIAANNAFEWVADKDNENVVKLVSDAQGRFEITGLLAGTYYLEETKQPAGYALLTSRQKFEVTATSYSATGQGIEYTAGSGKDDATKVVNKKITIPQTGGIGTIIFAVAGAAIMGIAVYAYVKNNKDEDQLA

SEQ ID NO: 5 - DNA, 1182 bp, Streptococcus pneumoniae TIGR4
atgattagtcgtatcttctttgttatggctctgtgtttttctcttgtatggggtgcacatgcagtccaagcgcaagaagatcacacgttggtcttgcaattggagaactatcaggaggtggttagtcaattgccatctcgtgatggtcatcggttgcaagtatggaagttggatgattcgtattcctatgatgatcgggtgcaaattgtaagagacttgcattcgtgggatgagaataaactttcttctttcaaaaagacttcgtttgagatgaccttccttgagaatcagattgaagtatctcatattccaaatggtctttactatgttcgctctattatccagacggatgcggtttcttatccagctgaatttctttttgaaatgacagatcaaacggtagagcctttggtcattgtagcgaaaaaaacagatacaatgacaacaaaggtgaagctgataaaggtggatcaagaccacaatcgcttggagggtgtcggctttaaattggtatcagtagcaagagatgtttctgaaaaagaggttcccttgattggagaataccgttacagttcttctggtcaagtagggagaactctctatactgataaaaatggagagatttttgtgacaaatcttcctcttgggaactatcgtttcaaggaggtggagccactggcaggctatgctgttacgacgctggatacggatgtccagctggtagatcatcagctggtgacgattacggttgtcaatcagaaattaccacgtggcaatgttgactttatgaaggtggatggtcggaccaatacctctcttcaaggggcaatgttcaaagtcatgaaagaagaaagcggacactatactcctgttcttcaaaatggtaaggaagtagttgtaacatcagggaaagatggtcgtttccgagtggaaggtctagagtatgggacatactatttatgggagctccaagctccaactggttatgttcaattaacatcgcctgtttcctttacaatcgggaaagatactcgtaaggaactggtaacagtggttaaaaataacaagcgaccacggattgatgtgccagatacaggggaagaaaccttgtatatcttgatgcttgttgccattttgttgtttggtagtggttattatcttacgaaaaaaccaaataactga

SEQ ID NO: 6 - protein, 393 aa, Streptococcus pneumoniae TIGR4
MISRIFFVMALCFSLVWGAHAVQAQEDHTLVLQLENYQEVVSQLPSRDGHRLQVWKLDDSYSYDDRVQIVRDLHSWDENKLSSFKKTSFEMTFLENQIEVSHIPNGLYYVRSIIQTDAVSYPAEFLFEMTDQTVEPLVIVAKKTDTMTTKVKLIKVDQDHNRLEGVGFKLVSVARDVSEKEVPLIGEYRYSSSGQVGRTLYTDKNGEIFVTNLPLGNYRFKEVEPLAGYAVTTLDTDVQLVDHQLVTITVVNQKLPRGNVDFMKVDGRTNTSLQGAMFKVMKEESGHYTPVLQNGKEVVVTSGKDGRFRVEGLEYGTYYLWELQAPTGYVQLTSPVSFTIGKDTRKELVTVVKNNKRPRIDVPDTGEETLYILMLVAILLFGSGYYLTKKPNN

SEQ ID NO: 7 - protein, 20 aa, Streptococcus pneumoniae TIGR4
LAGAEFVIANADNAGQYLAR

SEQ ID NO: 8 - protein, 5 aa, Artificial Sequence
  Synthetically generated peptide
YPXTG

SEQ ID NO: 9 - protein, 5 aa, Artificial Sequence
  Synthetically generated peptide
IPXTG

SEQ ID NO: 10 - protein, 5 aa, Artificial Sequence
  Synthetically generated peptide
VPXTG

SEQ ID NO: 11 - protein, 5 aa, Artificial Sequence
  Synthetically generated peptide
LPXTG

SEQ ID NO: 12 - protein, 5 aa, Artificial Sequence
  Synthetically generated peptide
VVXTG

SEQ ID NO: 13 - protein, 5 aa, Artificial Sequence
  Synthetically generated peptide
EVXTG

SEQ ID NO: 14 - protein, 5 aa, Artificial Sequence
  Synthetically generated peptide
QVXTG

SEQ ID NO: 15 - protein, 5 aa, Artificial Sequence
  Synthetically generated peptide
LPXAG

SEQ ID NO: 16 - protein, 5 aa, Artificial Sequence
  Synthetically generated peptide
QVPTG

SEQ ID NO: 17 - protein, 5 aa, Artificial Sequence
  Synthetically generated peptide
FPXTG

SEQ ID NO: 18 - DNA, 32 bp, Artificial Sequence
  Primer
tttttgggcccttcgtgttcgtgctgacttgc

SEQ ID NO: 19 - DNA, 33 bp, Artificial Sequence
  Primer
tttttggatccgatgttgctgattaagacgagc

SEQ ID NO: 20 - DNA, 21 bp, Artificial Sequence
  Primer
aacttcttttacgtttccgcc

SEQ ID NO: 21 - DNA, 19 bp, Artificial Sequence
  Primer
accgaaagacagacgagcc

SEQ ID NO: 22 - DNA, 32 bp, Artificial Sequence
  Primer
ttggatccctttaaatactgtagaaaagagga

SEQ ID NO: 23 - DNA, 31 bp, Artificial Sequence
  Primer
ttgggccctaaaacaattcatccagtaaaat

SEQ ID NO: 24 - DNA, 33 bp, Artificial Sequence
  Primer
tctatgcctattccagaggaaatggatcggatc

SEQ ID NO: 25 - DNA, 28 bp, Artificial Sequence
  Primer
ctagggccctttccttatgcttttggac

SEQ ID NO: 26 - DNA, 22 bp, Artificial Sequence
  Primer
aggagacattccttccgtatct

SEQ ID NO: 27 - DNA, 20 bp, Artificial Sequence
  Primer
caagagcacagcgtggtgct

SEQ ID NO: 28 - DNA, 21 bp, Artificial Sequence
  Primer
caaggtccaaacctactgaac

SEQ ID NO: 29 - DNA, 29 bp, Artificial Sequence
  Primer
gcgggcccctgagatatacagcacagtcc

SEQ ID NO: 30 - DNA, 29 bp, Artificial Sequence
  Primer
cgggatccctggcatttctgggaatcctg

SEQ ID NO: 31 - DNA, 23 bp, Artificial Sequence
  Primer
cgtttcaagtgctatcactgttc

SEQ ID NO: 32 - DNA, 26 bp, Artificial Sequence
  Primer
atataacatgaacagttgggttcttg

SEQ ID NO: 33 - DNA, 33 bp, Artificial Sequence
  Primer
atatagggcccaacctcttgcaattataccaca

SEQ ID NO: 34 - DNA, 32 bp, Artificial Sequence
  Primer
atataggatcccgcgtttgaactgtacctcaa

SEQ ID NO: 35 - DNA, 27 bp, Artificial Sequence
  Primer
atatacagtaactgtctcatccaaatc

SEQ ID NO: 36 - DNA, 29 bp, Artificial Sequence
  Primer
atatactgcttcaatccattagttatttc

SEQ ID NO: 37 - DNA, 30 bp, Artificial Sequence
  Primer
atatattgattgtaaaaattccatctatag

SEQ ID NO: 38 - DNA, 30 bp, Artificial Sequence
  Primer
ttggatccttatttccctcgtagtaaacgt

SEQ ID NO: 39 - DNA, 32 bp, Artificial Sequence
  Primer
ttgggcccaaagaaatgaaaggaaagctaagg

SEQ ID NO: 40 - DNA, 21 bp, Artificial Sequence
  Primer
caaggtccaaacctactgaac

SEQ ID NO: 41 - DNA, 29 bp, Artificial Sequence
  Primer
gcgggcccctgagatatacagcacagtcc

SEQ ID NO: 42 - DNA, 29 bp, Artificial Sequence
  Primer
cgggatccctggcatttctgggaatcctg

SEQ ID NO: 43 - DNA, 23 bp, Artificial Sequence
  Primer
cgtttcaagtgctatcactgttc

SEQ ID NO: 44 - DNA, 22 bp, Artificial Sequence
  Primer
gccccatcttgccctcactgcg

SEQ ID NO: 45 - DNA, 26 bp, Artificial Sequence
  Primer
atataacatgaacagttgggttcttg

SEQ ID NO: 46 - DNA, 33 bp, Artificial Sequence; identical to SEQ ID NO: 33
  Primer

SEQ ID NO: 47 - DNA, 32 bp, Artificial Sequence; identical to SEQ ID NO: 34
  Primer

SEQ ID NO: 48 - DNA, 27 bp, Artificial Sequence
  Primer
atatacagtaactgtctcatccaaatc

SEQ ID NO: 49 - DNA, 29 bp, Artificial Sequence
  Primer
atatactgcttcaatccattagttatttc

SEQ ID NO: 50 - DNA, 30 bp, Artificial Sequence; identical to SEQ ID NO: 37
  Primer

SEQ ID NO: 51 - DNA, 43 bp, Artificial Sequence
  Primer
cgcggatccaaaggagaatcatcatgctaaacaaatacattga

SEQ ID NO: 52 - DNA, 30 bp, Artificial Sequence
  Primer
ccctctagattataacaaatagtgagcctt

SEQ ID NO: 53 - protein, 6 aa, Artificial Sequence
  Synyhetically generated peptide
GSGGGG

SEQ ID NO: 54 - DNA, 6 bp, Artificial Sequence
  Examplary motif
gtcgtt

SEQ ID NO: 55 - DNA, 6 bp, Artificial Sequence
  Examplary motif
ttcgtt

SEQ ID NO: 56 - protein, 13 aa, Artificial Sequence
  Synthetically generated peptide
AGTTTTSVTVHXL

SEQ ID NO: 57 - protein, 5 aa, Artificial Sequence
  Synthetically generated peptide
LPXSG

SEQ ID NO: 58 - protein, 19 aa, Artificial Sequence
  Synthetically generated peptide
WLQDVHVYPKHQXXXXXXK

SEQ ID NO: 59 - protein, 19 aa, Artificial Sequence
  Synthetically generated peptide
WNYNVVAYPKNTXXXXXXK

SEQ ID NO: 60 - protein, 19 aa, Artificial Sequence
  Synthetically generated peptide
WLYDVNVFPKNGXXXXXXK

SEQ ID NO: 61 - protein, 19 aa, Artificial Sequence
  Synthetically generated peptide
WIYDVHVYPKNEXXXXXXK

SEQ ID NO: 62 - protein, 19 aa, Artificial Sequence
  Synthetically generated peptide
WNYNVHVYPKNTXXXXXXK

SEQ ID NO: 63 - protein, 19 aa, Artificial Sequence
  Synthetically generated peptide
FLSEINIYPKNVXXXXXXK

SEQ ID NO: 64 - protein, 19 aa, Artificial Sequence
  Synthetically generated peptide
DVVDAHVYPKNTXXXXXXK

SEQ ID NO: 65 - protein, 19 aa, Artificial Sequence
  Consensus sequence
XXXXXXXXPKXXXXXXXXX

SEQ ID NO: 66 - protein, 12 aa, Artificial Sequence
  Synthetically generated peptide
FCLVETATASGY

SEQ ID NO: 67 - protein, 12 aa, Artificial Sequence
  Synthetically generated peptide
FCLKETKAPAGY

SEQ ID NO: 68 - protein, 12 aa, Artificial Sequence
  Synthetically generated peptide
YVLVETEAPTGF

SEQ ID NO: 69 - protein, 12 aa, Artificial Sequence
  Synthetically generated peptide
YCLVETKAPYGY

SEQ ID NO: 70 - protein, 12 aa, Artificial Sequence
  Synthetically generated peptide
YKLKETKAPYGY

SEQ ID NO: 71 - protein, 12 aa, Artificial Sequence
  Synthetically generated peptide
YPITEEVAPSGY

SEQ ID NO: 72 - protein, 12 aa, Artificial Sequence
  Synthetically generated peptide
YRLFENSEPAGY

SEQ ID NO: 73 - protein, 12 aa, Artificial Sequence
  Synthetically generated peptide
YYLWELQAPTGY

SEQ ID NO: 74 - protein, 12 aa, Artificial Sequence
  Synthetically generated peptide
YYLEETKQPAGY

SEQ ID NO: 75 - protein, 12 aa, Artificial Sequence
  Consensus sequence
XXXXETXXXXGX

SEQ ID NO: 76 - protein, 10 aa, Artificial Sequence
  Synthetically generated peptide
WXXXVXVYPK

SEQ ID NO: 77 - protein, 4 aa, Artificial Sequence
  Synthetically generated peptide
LXET

(19) United States
(12) Patent Application Publication
(10) Pub. No.: US 2011/0275132 A1
(43) Pub. Date: Nov. 10, 2011

(54) PURIFICATION OF BACTERIAL ANTIGENS

(75) Inventors: Antonello COVACCI, Siena (IT); Markus HILLERINGMANN, Siena (IT); Ilaria FERLENGHI, Siena (IT)

(73) Assignee: NOVARTIS AG, Basel (CH)

(21) Appl. No.: 13/116,241

(22) Filed: May 26, 2011

Related U.S. Application Data
(63) Continuation of application No. 12/411,730, filed on Mar. 26, 2009, now abandoned.
Application No. 12/411,730 is a continuation of application No. 11/707,433, filed on Feb. 16, 2007, now abandoned.
(60) Provisional application No. 60/774,450, filed on Feb. 17, 2006.

(51) Int. Cl. C12N 13/00; C12N 1/06; C07K 14/315

(52) U.S. Cl. 435/173.7; 530/350; 435/259

(57) ABSTRACT

Presented are methods of isolation of pili and pilus-like structures from Gram-positive bacteria including Streptococcus pneumoniae and compositions that include such isolated pili. These compositions are useful as immunogenic compositions for the production of antibodies and immunostimulation. Also presented are methods of inhibiting Streptococcus pneumoniae, and methods of identifying inhibitors of Streptococcus pneumoniae.

This application is a continuation of Ser. No. 12/411,730 filed Mar. 26, 2009, which is a continuation of Ser. No. 11/707,433 filed Feb. 16, 2007, which claims the benefit of Ser. No. 60/774,450 filed Feb. 17, 2006. The contents of each of these applications are incorporated herein by reference in their entireties.

This application incorporates by reference the contents of a 39.3 kb text file created on Mar. 26, 2009 and named “52042_sequence_listing.txt,” which is the sequence listing for this application.

### FIELD OF THE INVENTION

The present invention relates to pili obtained from Gram-positive bacteria including Streptococcus pneumoniae, methods of producing and isolating the pili and the use of the pili for inducing an immune response against Gram-positive bacteria. The present invention also provides, inter alia, methods of detecting Gram-positive bacterial infection, methods of treating Gram-positive bacterial infection, and methods of identifying inhibitors of Gram-positive bacterial pili binding to a substrate. Antibodies which bind to the pili are also provided.

### BACKGROUND

The Gram-positive bacterium Streptococcus pneumoniae (also known as pneumococcus) is a major cause of morbidity and mortality world-wide and represents one of the four major infectious disease killers, together with HIV, malaria, and tuberculosis (1-5). It is a main cause of respiratory tract infections such as otitis media, sinusitis, and community acquired pneumonia, but also an important pathogen in invasive diseases such as septicemia and meningitis. Even though pneumococcus is a devastating pathogen, it also harmlessly colonizes healthy children attending day-care centers to a high extent (6, 7). A major virulence factor in pneumococcal disease is the polysaccharide capsule, by which pneumococci are grouped into at least ninety different serotypes (8). Other genetic factors, such as CbpA (choline-binding protein A) and pneumolysin, have been described to be of importance for virulence (9-11).

Infection by S. pneumoniae leads to invasive disease triggered by initial colonization of the nasopharynx, but the mechanisms of adhesion are not well understood. In vitro adhesion of encapsulated pneumococci is much lower than for nonencapsulated nonvirulent derivatives (4), even though capsule expression is essential for successful colonization of the upper airways. These observations suggest that in vivo, pneumococci are adhesive despite the production of a thick capsule (5).

In other Gram-positive bacteria, such as Corynebacterium diphtheriae (12, 13), Actinomyces spp. (14), and recently group A streptococci (GAS) and group B streptococci (GBS) (15, 16), pili-like surface structures have been identified by electron microscopy and characterized genetically as well as biochemically (12, 13, 15, 16). In Actinomyces spp. type 1 fimbrial genes mediate adhesion to dental and mucosal surfaces (17). However, there is a need for functional data on the physiological role and function in infectious disease of pili in pathogenic Streptococcus spp.

Gram-positive pili are extended polymers formed by a transpeptidase reaction involving covalent cross-linking subunit proteins containing specific amino acid motifs, which are assembled by specific sortases. Sortases are also responsible for covalent attachment of the pilus to the peptidoglycan cell wall.

### SUMMARY OF THE INVENTION

The present disclosure describes, inter alia, the isolation and characterization of pili from the Gram-positive bacterium Streptococcus pneumoniae. Pili play roles in the pathogenesis of S. pneumoniae and other Gram-positive bacteria and are useful, inter alia, in methods of treatment for and immunization against Gram-positive bacterial infections.

In some aspects, the disclosure provides isolated Gram-positive bacterial pili, e.g., Streptococcus pneumoniae pili, group A streptococcus (GAS) pili, or group B streptococcus (GBS) pili. In some embodiments, the pili comprise at least one of a S. pneumoniae RrgA protein, a S. pneumoniae RrgB protein and a S. pneumoniae RrgC protein, e.g., a polypeptide having the amino acid sequence of SEQ ID NO:2, SEQ ID NO:4, or SEQ ID NO:6, or a processed form thereof. In some embodiments, the isolated pili have a molecular weight from about 1×10⁵ to 1×10⁷ Da, or, in some embodiments, from 2×10⁶ to 3×10⁶ Da. In some embodiments, the isolated pili have a filament length from about 0.1 to 2 μm (e.g., about 0.1, 0.2, 0.5, 1, 1.5 or 2 μm). In some embodiments, the isolated pili have a diameter of about 10 nm (e.g., about 8, 9, 10, 11, or 12 nm). In some embodiments, the isolated pili comprise three protofilaments.

In some embodiments, the pili are separated from cells by enzymatic digestion (e.g., with one or more lytic enzymes such as peptidoglycan hydrolases (e.g., mutanolysin, lysostaphin, and lysozyme)). In some embodiments, the pili are separated from cells by mechanical shearing (e.g., by ultrasonication). In some embodiments, the pili are separated from cells by decreasing or inhibiting SrtA activity. In some embodiments, the pili are separated from cells by treating the cells with a compound that interferes with cell wall integrity (e.g., an antibiotic). In some embodiments, the pili are substantially free of bacterial cells. In some embodiments, the pili are substantially free of peptidoglycans. In some embodiments, the disclosure features methods of producing the isolated Gram-positive bacterial pili (e.g., S. pneumoniae pili), wherein the methods include subjecting a bacterial cell that produces Gram-positive bacterial pili (e.g., S. pneumoniae pili) to enzymatic digestion or mechanical shearing and isolating the pili from the cell.

In some aspects, the disclosure features immunogenic compositions that comprise one or more of the isolated Gram-positive bacterial pili (e.g., S. pneumoniae pili).

In some aspects, the disclosure features methods of isolating Gram-positive bacterial pili (e.g., S. pneumoniae, GAS, or GBS pili), wherein the methods comprise separating pili from bacterial cells that produce Gram-positive bacterial pili (e.g., Gram-positive bacterial cells or bacterial cells transformed to produce Gram-positive pili) and isolating the pili from the cells. In some embodiments, the pili are separated from cells by enzymatic digestion (e.g., with one or more lytic enzymes such as peptidoglycan hydrolases (e.g., mutanolysin, lysostaphin, and lysozyme). In some embodiments, the pili are separated from cells by mechanical shearing (e.g., by ultrasonication). In some embodiments, the pili are separated from cells by decreasing or inhibiting SrtA activity. In some embodiments, the pili are separated from cells by treating the cells with a compound that interferes with cell wall integrity (e.g., an antibiotic). In some embodiments, isolating comprises use of a density gradient centrifugation. In some embodiments, the isolating comprises reduction of polydispersity, such as separating components by size, e.g., using gel filtration chromatography. In some embodiments, the isolating includes one or more chromatography steps, e.g., gel filtration chromatography, ion-exchange chromatography, reverse phase chromatography, or affinity chromatography. In some embodiments, the method further comprises one or more concentrating steps.

In some aspects, the disclosure features antibodies that bind specifically to an isolated Gram-positive bacterial pilus (e.g., a S. pneumoniae pilus). In some embodiments, the antibodies are monoclonal antibodies, polyclonal antibodies, chimeric antibodies, human antibodies, humanized antibodies, single-chain antibodies, or Fab fragments. In some embodiments, the antibodies are labeled, e.g., with an enzyme, radioisotope, toxin, contrast agent (e.g., a gold particle), or fluorophore. In some embodiments, the antibodies bind preferentially to an isolated bacterial pilus or a fragment thereof, as compared to binding of the antibodies to the individual proteins that make up the pilus. In some embodiments, the antibodies preferentially bind to a pilus complex as compared to the binding of the antibody to an uncomplexed pilus protein selected from the group consisting of RrgA, RrgB, and RrgC. In some embodiments, the antibodies do not bind specifically to uncomplexed RrgA, RrgB, or RrgC.

In some aspects, the disclosure features methods of inducing an immune response against a Gram positive bacterium (e.g., S. pneumoniae), wherein the methods include administering an effective amount of Gram-positive bacterial pili, e.g., S. pneumoniae pili (e.g., isolated S. pneumoniae pili), to a subject, e.g., a human or non-human animal.

In some aspects, the disclosure features methods of detecting a Gram-positive bacterial infection (e.g., a S. pneumoniae infection) in a subject, e.g., a human, wherein the methods include assaying a sample from the subject, e.g., serum or sputum, for evidence of the presence of Gram-positive bacterial pili (e.g., S. pneumoniae pili). In some embodiments, evidence of presence of Gram-positive bacterial pili (e.g., S. pneumoniae pili) is provided by the presence of an antibody to Gram-positive bacterial pili (e.g., S. pneumoniae pili). In some embodiments, the antibody preferentially binds to a pilus complex as compared to the binding of the antibody to an uncomplexed pilus protein (e.g., RrgA, RrgB, and RrgC). In some embodiments, the antibody does not bind specifically to an uncomplexed pilus protein (e.g., RrgA, RrgB, or RrgC).

In some aspects, the disclosure features methods of detecting a Gram-positive bacterial infection, e.g., a S. pneumoniae infection, in a subject, wherein the methods include contacting a sample with an agent (e.g., an antibody) that binds specifically to a Gram-positive bacterial pilus, e.g., a S. pneumoniae pilus, and detecting binding of the agent to a component of the sample. In some embodiments, the antibody preferentially binds to a pilus complex as compared to the binding of the antibody to an uncomplexed pilus protein (e.g., RrgA, RrgB, and RrgC). In some embodiments, the antibody does not bind specifically to an uncomplexed pilus protein (e.g., RrgA, RrgB, or RrgC).

In some aspects, the disclosure features methods of treating a subject (e.g., a human subject) having or suspected of having a Gram-positive bacteria (e.g., S. pneumoniae) infection, wherein the methods include administering to the subject an effective amount of an agent that binds specifically to Gram-positive pili. In some embodiments, the agent is an antibody (e.g., a monoclonal antibody, a polyclonal antibody, a chimeric antibody, a human antibody, a humanized antibody, a single-chain antibody, or a Fab fragment). In some embodiments, the agent (e.g., an antibody) blocks attachment or binding of Gram-positive bacteria to cells such as host cells. The cells can be epithelial cells, e.g., lung or nasopharyngeal epithelia cells. In some embodiments, the antibody binds preferentially to an isolated bacterial pilus or a fragment thereof, as compared to the individual proteins that make up the pilus. In some embodiments, the agent (e.g., an antibody) binds specifically to one or more S. pneumoniae pili proteins, e.g., RrgA, RrgB, or RrgC (e.g., one or more polypeptides having the amino acid sequence of SEQ ID NOs:2, 4, or 6, or processed forms of any thereof). In some embodiments, the agent (e.g., an antibody) specifically binds to a polypeptide having amino acid residues 316-419 of SEQ ID NO:4. In some embodiments the agent (e.g., an antibody) blocks at least 50% of S. pneumoniae attachment to A549 lung epithelial cells as compared to a control, as measured in an attachment assay.

In some aspects, the disclosure features methods of determining a course of treatment for a subject (e.g., a human subject) having or suspected of having a Gram-positive bacterial (e.g., S. pneumoniae) infection, wherein the methods include assaying a sample from the subject for the presence of an antibody to Gram-positive pili and choosing a course of treatment based on the presence or absence of the antibody. The method can further include treating the subject with an antibiotic agent if the presence of the antibody is not detected. The method can also include treating the subject with an anti-inflammatory agent if the presence of the antibody is detected.

The disclosure also features isolated Gram-positive pili that include polypeptides that include an amino acid sequence of a Gram-positive (e.g., S. pneumoniae) pilus protein with up to 50 (e.g., up to 40, 30, 20, 10, or 5) amino acid substitutions, insertions, or deletions. In some embodiments, the amino acid substitutions are conservative amino acid substitutions. In some embodiments, the Gram-positive pilus protein is RrgA (e.g., SEQ ID NO:2), RrgB (e.g., SEQ ID NO:4), or RrgC (e.g., SEQ ID NO:6). In some embodiments, the polypeptides include the amino acid sequences of two or more of SEQ ID NOs:2, 4, or 6, or immunogenic fragments of any thereof In some embodiments, the polypeptides include the amino acid sequences of SEQ ID NOs:2, 4, and 6, or immunogenic fragments of all thereof. The disclosure also features immunogenic fragments of isolated Gram-positive pili, e.g., those containing S. pneumoniae pilus proteins such as RrgA, RrgB, and RrgC (e.g., immunogenic fragments of SEQ ID NOs:2, 4, and 6). Also featured in the disclosure are methods of inducing an immune response against a Gram positive bacterium (e.g., S. pneumoniae), wherein the methods include administering an effective amount of an isolated Gram-positive pilus to a subject, e.g., a human subject. The disclosure also features methods of producing isolated Gram-positive pili by transforming a host cell with one or more nucleic acids sufficient to produce the pili, and isolating the pili from the host cell.

In some aspects, the disclosure features methods of expressing an anti-Gram-positive (e.g., S. pneumoniae) pilus antibody in a cell, wherein the methods include expressing a nucleic acid encoding the anti- Gram-positive pilus antibody in the cell.

In some aspects, the disclosure features methods of purifying Gram-positive (e.g., S. pneumoniae) bacteria from a sample that includes the Gram-positive bacteria, wherein the methods include providing an affinity matrix that includes an antibody that binds specifically to a Gram-positive pilus bound to a solid support; contacting the sample with the affinity matrix to form an affinity matrix/Gram-positive bacterium complex; separating the affinity matrix/Gram-positive bacterium complex from the remainder of the sample; and releasing the Gram-positive bacterium from the affinity matrix.

In some aspects, the disclosure features methods of delivering a cytotoxic agent or a diagnostic agent to a Gram-positive bacterium (e.g., S. pneumoniae), wherein the methods include providing the cytotoxic agent or the diagnostic agent conjugated to an antibody or fragment thereof of that binds specifically to a Gram-positive (e.g., S. pneumoniae) pilus; and exposing the bacterium to the antibody-agent or fragment-agent conjugate.

In some aspects, the disclosure features methods of identifying modulators of S. pneumoniae, wherein the methods include contacting a cell susceptible to S. pneumoniae infection, e.g., a HEP2 cell, CHO cell, HeLa cell, or A549 lung epithelia cell, with a candidate compound and S. pneumoniae, and determining whether a S. pneumoniae activity, e.g., attachment to a cell (e.g., an A549 lung epithelial cell), is inhibited, wherein inhibition of the S. pneumoniae activity is indicative of a S. pneumoniae inhibitor.

In some aspects, the disclosure features methods of identifying modulators of Gram-positive (e.g., S. pneumoniae) pili binding, wherein the methods include contacting an animal cell susceptible to Gram-positive pili binding with a candidate compound and a bacterial cell having Gram-positive pili, and determining whether binding of the bacterial cell to the animal cell is inhibited, wherein inhibition of the binding activity is indicative of an inhibitor of Gram-positive pili binding.

In some aspects, the disclosure features methods of identifying modulators of Gram-positive (e.g., S. pneumoniae) pili binding, wherein the methods include contacting a cell susceptible to Gram-positive pili binding with a candidate compound and Gram-positive pili, and determining whether binding of the pili to the cell is inhibited, wherein inhibition of the binding activity is indicative of an inhibitor of Gram-positive pili binding.

In some aspects, the disclosure features methods of identifying modulators of Gram-positive (e.g., S. pneumoniae) pili binding, said method comprising contacting a cell susceptible to Gram-positive pili binding with a candidate compound and a Gram-positive pilus protein or cell-binding fragment thereof, and determining whether binding of the pilus protein or fragment thereof to the cell is inhibited, wherein inhibition of the binding activity is indicative of an inhibitor of Gram-positive pili binding.

In some aspects, the disclosure features methods of identifying modulators of Gram-positive (e.g., S. pneumoniae) pili binding, said method comprising contacting a protein susceptible to Gram-positive pili binding, e.g., an extracellular matrix protein or Gram-positive pilus-binding fragment thereof with a candidate compound and a Gram-positive pilus, Gram-positive pilus protein, or a fragment thereof, and determining whether binding between the two proteins or fragments thereof is inhibited, wherein inhibition of the binding activity is indicative of an inhibitor of Gram-positive pili binding.

The disclosure also features pharmaceutical, immunogenic, and vaccine compositions that include isolated Gram-positive bacterial pili (e.g., S. pneumoniae pili). The disclosure also features the use of Gram-positive (e.g., S. pneumoniae) pili (or any of the polypeptides or nucleic acids described above) for the preparation of an immunogenic composition or a vaccine composition for the treatment or prophylaxis of Gram-positive bacterial infection. The disclosure also features Gram-positive (e.g., S. pneumoniae) pili (or any of the polypeptides or nucleic acids described above) for use in medicine. The disclosure also features Gram-positive (e.g., S. pneumoniae) pili (or any of the polypeptides or nucleic acids described above) for use in treating or preventing Gram-positive bacterial infection.

The disclosure also features pharmaceutical compositions that include agents (e.g., antibodies) that bind specifically to S. pneumoniae pili. The disclosure also features the use of agents (e.g., antibodies) that bind specifically to S. pneumoniae pili for the preparation of a medicament for the treatment or prophylaxis of S. pneumoniae infection. The disclosure also features such agents for use in medicine. The disclosure also features such agents for use in treating or preventing Gram-positive bacterial infection.

The disclosure also features methods of isolating Streptococcus pneumoniae pili, wherein the methods include separating pili from S. pneumoniae cells that produce S. pneumoniae pili, e.g., S. pneumoniae TIGR4, and isolating S. pneumoniae pili. In some embodiments, the pili are separated from S. pneumoniae cells by enzymatic digestion (e.g., with one or more lytic enzymes such as peptidoglycan hydrolases (e.g., mutanolysin, lysostaphin, and lysozyme). In some embodiments, the pili are separated from S. pneumoniae cells by mechanical shearing (e.g., by ultrasonication). In some embodiments, the pili are separated from S. pneumoniae cells by decreasing or inhibiting SrtA activity. In some embodiments, the pili are separated from S. pneumoniae cells by treating the cells with a compound that interferes with cell wall integrity (e.g., an antibiotic). In some embodiments, the methods include degrading nucleic acids with a nuclease. In some embodiments, the methods include reduction of polydispersity, such as by separating S. pneumoniae pili by size using gel filtration chromatography. In some embodiments, the methods include one or more chromatography steps, e.g.; gel filtration chromatography, ion-exchange chromatography, reverse phase chromatography, or affinity chromatography. In some embodiments, the S. pneumoniae cells that produce S. pneumoniae pili express more pili than S. pneumoniae TIGR4.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the additional embodiments below.

### BRIEF DESCRIPTION OF THE DRAWINGS

FIG. 1. (A) Negative staining of S. pneumoniae strain T4 showing abundant pili on the bacterial surface. (B) Negative staining of mutant strain T4Δ(rrgA-srtD) showing no pili. (C) Negative staining of the T4Δ(mgrA) mutant showing abundant pili. (D) Negative staining of the T4Δ(rrgA-srtD, mgrA) mutant showing no pili on the bacterial surface. (E) Immunogold labeling of T4 by using anti-RrgA. (F) Immunogold labeling of T4 with anti-RrgB (5 nm) and anti-RrgC (10 nm). Anti-RrgB was shown to decorate entire pili (bar, 200 nm). (G) High magnification of T4 pili double-labeled with anti-RrgB (5 nm) and anti-RrgC (10 nm). It shows specific labeling of a pilus by anti-RrgC as indicated by arrows (bar, 100 nm). (H) Immunogold labeling of the deletion mutant S. pneumoniae T4Δ(rrgA-srtD) with no visible pili on the surface detectable by anti-RrgB- and anti-RrgC (bar, 200 nm).

FIG. 2. Genome organization of the rlrA islet in serotype 4 strain T4 (TIGR4) and comparison with the laboratory strain R6 from available sequences. The 19F strain, ST162^{19F}, shares a similar organization with an overall 98% sequence identity, whereas the nonencapsulated strain R6 and its progenitor D39 are pilus-islet-negative strains. Insertion sequences (IS1167) flank the locus in positive strains [one of the transposases is frame-shifted (fs)], whereas an RUP element (repeat unit in pneumococcus) is identified in the pilus-islet-negative strain. The size of the locus, as well as its relative G+C content, is shown. The position of the negative regulator mgrA is indicated. Included for comparison is the genome organization of the islets encoding pilus structures in Streptococcus agalactiae and Corynebacterium diphtheriae.

FIG. 3. (A) Western blot using a 4-12% polyacrylamide gradient gel with the RrgB antiserum detects a ladder of high molecular weight (HMW) polymers in strains expressing pili (T4, T4Δ(mgrA), ST162^{19F}, and ST16219FΔ(mgrA)), whereas the mutant strains lacking pili (T4Δ(rrgA-srtD), T4Δ(rrgA-srtD, mgrA), and ST162^{19F}Δ(rrgA-srtD)) have no HMW polymers. The mgrA mutant shows an increased intensity when compared with the respective wild type. (B) Western blot with the RrgB antiserum using a 4-12% gradient gel for D39 lacking the islet, the mutant D39 with the rlrA islet introduced (D39∇(rrgA-srtD)), and its rlrA deletion derivative (D39∇(rrgA-srtD)A(rlrA)).

FIG. 4. (A) Adherence of D39 and D39∇(rrgA-srtD), as well as D39∇(rrgA-srtD)L(rlrA) to monolayers of A549 lung epithelial cells. (B-D) Immunofluorescence microscopy of D39 (B), D39∇(rrgA-srtD) (C), and D39∇(rrgA-srtD)Δ(rlrA) (D) adhering to A549 lung epithelial cells. Shown are labeling of pneumococci with anti-capsular antibody (green) and visualization of epithelial F-actin with rhodamine (red).

FIG. 5. (A-E) Intranasal challenge of C57BL/6 mice with piliated T4 and its isogenic nonpiliated deletion mutant T4Δ(rrgA-srtD). (A and B) Survival of mice after inoculation with 5×10⁶ cfu (high dose, A) or 5×10⁵ cfu (medium dose, B). Survival was analyzed by using the Kaplan-Meier log rank test. (C-E) In vivo competition infection experiments where T4 and its isogenic mutant T4Δ(rrgA-srtD) were mixed in a ratio of 1:1 before intranasal infection. The competitive index (CI) was calculated as described below; each circle represents the CI for one individual mouse in each set of competition experiments. A CI below 1 indicates a competitive disadvantage of the mutant in relation to the wild-type strain. CI values<10⁻⁴ were set to 10⁻⁴. All mice were colonized. (C) CI in colonization, pneumonia, and bacteremia after high-dose challenge (n=20). Of 20 mice, only 14 presented pneumonia (defined as bacteria recovered from the lungs), and 14 were bacteremic. (D) CI in colonization after medium dose challenge (n=10). Of 10 mice, only 5 presented pneumonia and only 1 was bacteremic. (E) CI in colonization after low dose challenge (n=10). Of 10 mice, only 4 presented pneumonia and none developed bacteremia. (F) CI in colonization and pneumonia after with mixed infection with wild-type D39 and its isogenic pilus islet insertion derivative D39∇(rrgA-srtD), or D39∇(rrgA-srtD)∇(rlrA) with the rlrA gene inactivated. A CI above 1 indicates a virulence gain by the presence of the rlrA islet in D39∇(rrgA-srtD).

FIG. 6. Role of the rlrA pilus islet in systemic host inflammatory response. Mice were challenged i.p. with high challenge dose (5×10⁶ to 2×10⁷ cfu) of T4, ST162^{19F}, and their isogenic mutants T4Δ(rrgA-srtD), and ST162^{19F}Δ(rrgA-srtD) and killed at 6 hours after infection. (A) Bacterial outgrowth in blood after high-dose i.p. challenge. Results from individual mice are shown. Horizontal lines represent the medians, and analysis by Mann-Whitney U test gives no significant differences (P>0.05). (B) Serum TNF response. Data are presented as means and SEMs. Statistical significance was established by Mann-Whitney U test (**, P<0.0001; *, P<0.001). (C and D) TNF response for individual mice correlated to the bacteremic levels after inoculation with T4 and T4Δ(rrgA-srtD) (C) or ST162^{19F} and ST1621^{9F}Δ(rrgA-srtD) (D).

FIG. 7. Analysis of the IL-6 response for the same i.p. challenges as shown in FIG. 6. Bacterial growth in blood is shown in FIG. 6A. (A) Serum IL-6 response at 6 hours after infection. Data are presented as means and SEMs (Mann-Whitney U test; *, P<0.0001). (B) IL-6 response for individual mice correlated to the bacteremia levels after inoculation with T4 and T4Δ(rrgA-srtD).

FIG. 8 is an analysis of the structural proteins RrgA, RrgB and RrgC of pneumococcal T4 pili. 8A is a schematic drawing of predicted motifs found in Gram positive pili proteins. 8B is a depiction of sequences of predicted pilin and E-box motifs in S. pneumoniae (T4), where present. Sequences of Corynebacterium sp. pilin and E-box motifs are shown for reference (Ton-That et al., 2004, Mol. Microbiol., 53:251-261; Ton-That and Schneewind, 2004, Trends Microbiol., 12:228-34; Scott and Zähner; 2006, Mol. Microbiol., 62:320-330). 8C is a summary of motifs found in pneumococcal T4 RrgA, RrgB and RrgC. 8A and 8C, S: N-terminal signal peptide, P: Pilin motif, E: E-box, C: cell wall sorting signal motif, M: hydrophobic stretch and charged tail.

FIG. 9A is depicts a polyacrylamide gel stained with Coomassie blue showing self-association of purified RrgA and RrgB proteins.

FIG. 9B depicts an immunoblot showing self-association of purified RrgA and RrgB proteins.

FIG. 9C depicts a series of traces of size exclusion chromatography of purified RrgA, RrgB, and RrgC proteins. Higher molecular weight complexes were observed for RrgA and RrgB.

FIG. 10A depicts a line graph depicting purification of high molecular weight, native, pneumococcal T4 pili by sucrose gradient.

FIG. 10B depicts a trace depicting purification of high molecular weight, native, pneumococcal T4 pili by size exclusion chromatography.

FIG. 10C depicts polyacrylamide gels showing results of the purification of high molecular weight, native, pneumococcal T4 pili. The gel on the left shows the results of silver staining. The gel on the right shows an immunoblot with antibody that binds specifically to RrgB.

FIG. 11A depicts the results of an Edmann analysis to determine the N-terminal amino acid sequence of pili proteins (underlined) as compared to the predicted amino acid sequence of RrgB. The N-terminus of the pili protein corresponds to the predicted signal peptidase cleavage site (/).

FIG. 11B depicts the results of a mass spectroscopy analysis of a tryptic digest of purified high molecular weight pili. A tryptic peptide sequence (italics) of high molecular weight pili (isolated from an SDS-PAGE gel) matches with the predicted RrgB amino acid sequence (bold).

FIG. 12 shows bacteremia and mortality of BALB/c mice immunized (IP) with antisera to HMW pili (50 μl/mouse) and challenged (IP) with 260 CFU of T4/mouse. A T4Δpilus preparation served as negative control. A. Bacteremia at 24 hours post-challenge. Circles=values of CFU per ml of blood of single animals; horizontal bars=geometric mean of each group; dashed line=detection limit (i.e., no CFU were detected in blood samples below dashed line). B. Mortality course. Diamonds=survival days of single animals, horizontal bars=median of survival days of each group; dashed line=endpoint of observation (i.e., animals above the dashed line survived at the endpoint). ctrl=mice receiving only the corresponding adjuvant plus saline; anti-pilus=antisera to purified HMW pili; anti-Δpilus=antisera to purified control (T4Δpilus); *=P<0.05 and **=P<0.01, in comparison with the corresponding control group.

FIG. 13 depicts a series of graphs showing results of binding of purified recombinant proteins (BSA, RrgA, RrgB, RrgC) and native pili to BSA and extracellular matrix proteins mucin I, hyaluronic acid, vitronectin, chondroitin sulfate, lactoferrin, collagens I and IV, laminin, Fibronectin and Fibrinogen. BSA served as negative control. Binding was quantified by ELISA at an absorbance of 405 nm.

FIG. 14 depicts a series of bar graphs showing induction of inflammatory cytokines TNF-alpha, IL-12p40, and IL-6 by peripheral blood mononuclear cells (PBMC) and monocytes challenged in vitro with purified pili and a delta pili control preparation.

FIG. 15 depicts an electron micrograph of a Streptococcus pneumoniae bacterium immunogold labeled with an antibody specific for RrgB.

FIG. 16 depicts an electron micrograph of a purified pili preparation immunogold labeled with antibodies specific for RrgA (conjugated to 15 nm gold particles), RrgB (conjugated to 5 nm gold particles), and RrgC (conjugated to 10 nm gold particles). RrgB is the major component of the pilus. RrgA and RrgC are found along the length of the pilus, RrgA often being found in clusters.

FIG. 17 depicts an electron micrograph of purified pili negatively stained with phosphotungstic acid (PTA) and viewed at 5000× magnification.

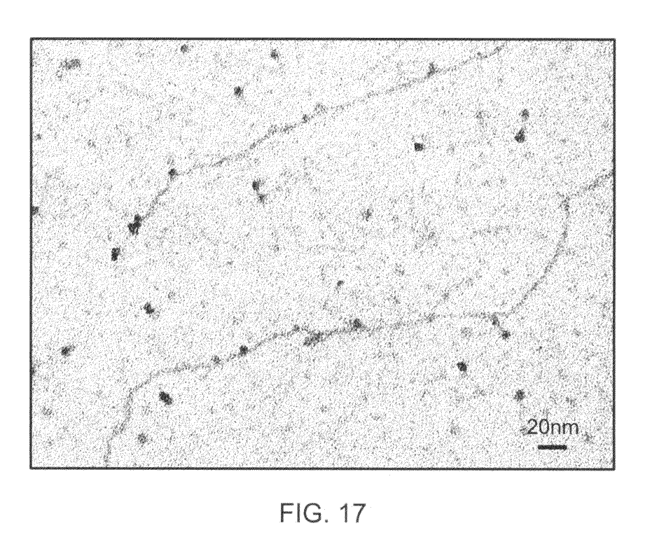

FIG. 18 is a schematic diagram of pili structural analysis to determine average pili diameter.

FIG. 19 is a schematic diagram of pili structural analysis to determine pili volume.

FIG. 20 is a schematic diagram of a method of generating an improved 2D representation of a pilus by averaging and filtering pilus electron micrographs.

FIG. 21 is a schematic diagram of rotated 2D views of a pilus showing a helical structure made up of three protofilaments.

FIG. 22 is a schematic diagram of determination of density profiles across pilus structure at two positions.

FIG. 23 depicts a model of a pilus structure. The pili are made by at least 3 “protofilaments” arranged in a coiled-coil structure with an average diameter of 10.5-11.0 nm and a pitch of 13.2 nm. The diameter of the pili at the node position is 6.8 nm, and every single “protofilament” has a diameter of 3.5 nm.

### DETAILED DESCRIPTION

Applicants have isolated and characterized pili from a Gram positive bacterium, Streptococcus pneumoniae (also known as pneumococcus). These pili were identified as expressed by S. pneumoniae TIGR4, a clinical, capsular serotype 4 isolate, the genome of which was sequenced by The Institute for Genomic Research (see worldwide web site tigr.org). These pili are encoded by a pathogenicity island, the rlrA islet, which is present in some but not all clinical pneumococcal isolates. The pili are shown to be important for pneumococcal adherence to lung epithelial cells as well as for colonization in a murine model of infection. Likewise, the pili are also shown to affect the development of pneumonia .and bacteremia in mice. Furthermore, pilus-expressing pneumococci evoked a higher tumor necrosis factor (TNF) response during systemic infections than nonpiliated isogenic mutants, indicating that the pili play a role in the host inflammatory response. Accordingly, this disclosure features, inter alia, Gram-positive bacterial (e.g., S. pneumoniae) pili and pilus protein compositions and use of the same in methods of treatment for and immunization against Gram-positive bacterial (e.g., S. pneumoniae) infections.

### Streptococcus Pneumoniae Pili

Pneumococcal pili are encoded by an rlrA islet present in S. pneumoniae TIGR4, containing 3 sortases and 3 genes coding for proteins containing LPXTG motifs (rrgA, rrgB, and rrgC). Immunogold labeling with antibodies against the RrgA, RrgB, and RrgC proteins detected elongated filament structures on the surface of S. pneumoniae. Anti-RrgA was shown to label the bacterial cell surface, suggesting that RrgA anchors the pilus structure to the cell wall. Anti-RrgB was shown to decorate the entire pili, whereas anti-RrgC was concentrated in the pili tips. Deletion of the pilus genes eliminated pili staining, whereas deletion of a negative regulator of the pilus operon (mgrA) gave an increased amount of pili on the cell surface. The cell surface location of S. pneumoniae pili make them attractive as antigens.

Pili were isolated to homogeneity or near homogeneity from S. pneumoniae TIGR4, and showed molecular masses ranging from 2×10⁶ to 3×10⁶ Da. Purified pili were present as elongated filaments up to about 1 μm long and about 10 nm in diameter. Immunogold labeling detected both RrgB and RrgC proteins in the isolated pili.

An exemplary rrgA nucleic acid sequence (TIGR Annotation No. sp0462) is hereby provided:

(SEQ ID NO: 1)
ATGCTTAACAGGGAGACACACATGAAAAAAGTAAGAAAGATATTTCAG
AAGGCAGTTGCAGGACTGTGCTGTATATCTCAGTTGACAGCTTTTTCT
TCGATAGTTGCTTTAGCAGAAACGCCTGAAACCAGTCCAGCGATAGGA
AAAGTAGTGATTAAGGAGACAGGCGAAGGAGGAGCGCTTCTAGGAGAT
GCCGTCTTTGAGTTGAAAAACAATACGGATGGCACAACTGTTTCGCAA
AGGACAGAGGCGCAAACAGGAGAAGCGATATTTTCAAACATAAAACCT
GGGACATACACCTTGACAGAAGCCCAACCTCCAGTTGGTTATAAACCC
TCTACTAAACAATGGACTGTTGAAGTTGAGAAGAATGGTCGGACGACT
GTCCAAGGTGAACAGGTAGAAAATCGAGAAGAGGCTCTATCTGACCAG
TATCCACAAACAGGGACTTATCCAGATGTTCAAACACCTTATCAGATT
ATTAAGGTAGATGGTTCGGAAAAAAACGGACAGCACAAGGCGTTGAAT
CCGAATCCATATGAACGTGTGATTCCAGAAGGTACACTTTCAAAGAGA
ATTTATCAAGTGAATAATTTGGATGATAACCAATATGGAATCGAATTG
ACGGTTAGTGGGAAAACAGTGTATGAACAAAAAGATAAGTCTGTGCCG
CTGGATGTCGTTATCTTGCTCGATAACTCAAATAGTATGAGTAACATT
CGAAACAAGAATGCTCGACGTGCGGAAAGAGCTGGTGAGGCGACACGT
TCTCTTATTGATAAAATTACATCTGATTCAGAAAATAGGGTAGCGCTT
GTGACTTATGCTTCCACTATCTTTGATGGGACCGAGTTTACAGTAGAA
AAAGGGGTAGCAGATAAAAACGGAAAGCGATTGAATGATTCTCTTTTT
TGGAATTATGATCAGACGAGTTTTACAACCAATACCAAAGATTATAGT
TATTTAAAGCTGACTAATGATAAGAATGACATTGTAGAATTAAAAAAT
AAGGTACCTACCGAGGCAGAAGACCATGATGGAAATAGATTGATGTAC
CAATTCGGTGCCACTTTTACTCAGAAAGCTTTGATGAAGGCAGATGAG
ATTTTGACACAACAAGCGAGACAAAATAGTCAAAAAGTCATTTTCCAT
ATTACGGATGGTGTCCCAACTATGTCGTATCCGATTAATTTTAATCAT
GCTACGTTTGCTCCATCATATCAAAATCAACTAAATGCATTTTTTAGT
AAATCTCCTAATAAAGATGGAATACTATTAAGTGATTTTATTACGCAA
GCAACTAGTGGAGAACATACAATTGTACGCGGAGATGGGCAAAGTTAC
CAGATGTTTACAGATAAGACAGTTTATGAAAAAGGTGCTCCTGCAGCT
TTCCCAGTTAAACCTGAAAAATATTCTGAAATGAAGGCGGCTGGTTAT
GCAGTTATAGGCGATCCAATTAATGGTGGATATATTTGGCTTAATTGG
AGAGAGAGTATTCTGGCTTATCCGTTTAATTCTAATACTGCTAAAATT
ACCAATCATGGTGACCCTACAAGATGGTACTATAACGGGAATATTGCT
CCTGATGGGTATGATGTCTTTACGGTAGGTATTGGTATTAACGGAGAT
CCTGGTACGGATGAAGCAACGGCTACTAGTTTTATGCAAAGTATTTCT
AGTAAACCTGAAAACTATACCAATGTTACTGACACGACAAAAATATTG
GAACAGTTGAATCGTTATTTCCACACCATCGTAACTGAAAAGAAATCA
ATTGAGAATGGTACGATTACAGATCCGATGGGTGAGTTAATTGATTTG
CAATTGGGCACAGATGGAAGATTTGATCCAGCAGATTACACTTTAACT
GCAAACGATGGTAGTCGCTTGGAGAATGGACAAGCTGTAGGTGGTCCA
CAAAATGATGGTGGTTTGTTAAAAAATGCAAAAGTOCTCTATGATACG
ACTGAGAAAAGGATTCGTGTAACAGGTCTGTACCTTGGAACGGATGAA
AAAGTTACGTTGACCTACAATGTTCGTTTGAATGATGAGTTTGTAAGC
AATAAATTTTATGATACCAATGGTCGAACAACCTTACATCCTAAGGAA
GTAGAACAGAACACAGTGCGCGACTTCCCGATTCCTAAGATTCGTGAT
GTGCGGAAGTATCCAGAAATCACAATTTCAAAAGAGAAAAAACTTGGT
GACATTGAGTTTATTAAGGTCAATAAAAATGATAAAAAACCACTGAGA
GGTGCGGTCTTTAGTCTTCAAAAACAACATCCGGATTATCCAGATATT
TATGGAGCTATTGATCAAAATGGCACTTATCAAAATGTGAGAACAGGT
GAAGATGGTAAGTTGACCTTTAAAAATCTGTCAGATGGGAAATATCGA
TTATTTGAAAATTCTGAACCAGCTGGTTATAAACCCGTTCAAAATAAG
CCTATCGTTGCCTTCCAAATAGTAAATGGAGAAGTCAGAGATGTGACT
TCAATCGTTCCACAAGATATACCAGCGGGTTACGAGTTTACGAATGAT
AAGCACTATATTACCAATGAACCTATTCCTCCAAAGAGAGAATATCCT
CGAACTGGTGGTATCGGAATGTTGCCATTCTATCTGATAGGTTGCATG
ATGATGGGAGGAGTTCTATTATACACACGGAAACATCCGTAA

An exemplary RrgA amino acid sequence (TIGR Annotation No. SP0462) is hereby provided:

(SEQ ID NO: 2)
MLNRETHMKKVRKIFQKAVAGLCCISQLTAFSSIVALAETPETSPAIG
KVVIKETGEGGALLGDAVFELKNNTDGTTVSQRTEAQTGEAIFSNIKP
GTYTLTEAQPPVGYKPSTKQWTVEVEKNGRTTVQGEQVENREEALSDQ
YPQTGTYPDVQTPYQIIKVDGSEKNGQHKALNPNPYERVIPEGTLSKR
IYQVNNLDDNQYGIELTVSGKTVYEQKDKSVPLDVVILLDNSNSMSNI
RNKNARRAERAGEATRSLIDKITSDSENRVALVTYASTIFDGTEFTVE
KGVADKNGKRLNDSLFWNYDQTSFTTNTKDYSYLKLTNDKNDIVELKN
KVPTEAEDHDONRLMYQFGATFTQKALMKADEILTQQARQNSQKVIFH
ITDGVPTMSYPINFNHATFAPSYQNQLNAFFSKSPNKDGILLSDFITQ
ATSGEHTIVRGDGQSYQMFTDKTVYEKGAPAAFPVKPEKYSEMKAAGY
AVIGDPINGGYIWLNWRESILAYPFNSNTAKITNHGDPTRWYYNGNIA
PDGYDVFTVGIGINGDPGTDEATATSFMQSISSKPENYTNVTDTTKIL
EQLNRYFHTIVTEKKSIENGTITDPMGELIDLQLGTDGRFDPADYTLT
ANDGSRLENGQAVGGPQNDGGLLKNAKVLYDTTEKRIRVTGLYLGTDE
KVTLTYNVRLNDEFVSNKFYDTNGRTTLHPKEVEQNTVRDFPIPKIRD
VRKYPEITISKEKKLGDIEFIKVNKNDKKPLRGAVFSLQKQHPDYPDI
YGAIDQNGTYQNVRTGEDGKLTFKNLSDGKYRLFENSEPAGYKPVQNK
PIVAFQIVNGEVRDVTSIVPQDIPAGYEFTNDKHYITNEPIPPKREYP
RTGGIGMLPFYLIGCMMMGGVLLYTRKHP

RrgA contains a sortase substrate motif YPXTG (SEQ ID NO:8), shown in underscore in SEQ ID NO:2, above. Two putative Cna protein B-type domains (Deivanayagam et al., 2000, Structure, 8:67-78) have been identified at amino acid residues 62-132 and 751-824 of SEQ ID NO:2. A putative von Willebrand factor type A domain has been identified (Sadler, 1998, Annu. Rev. Biochem., 67:395-424; Ponting et al., 1999, J. Mol. Biol., 289:729-4 226-579). This von Willebrand factor type A domain may be involved in mediating cell adhesion or cell signaling properties of S. pneumoniae pili.

An exemplary rrgB nucleic acid sequence (TIGR Annotation No. sp0463) is hereby provided:

(SEQ ID NO: 3)
ATGAAATCAATCAACAAATTTTTAACAATGCTTGCTGCCTTATTACTG
ACAGCGAGTAGCCTGTTTTCAGCTGCAACAGTTTTTGCGGCTGGGACG
ACAACAACATCTGTTACCGTTCATAAACTATTGGCAACAGATGGGGAT
ATGGATAAAATTGCAAATGAGTTAGAAACAGGTAACTATGCTGGTAAT
AAAGTGGGTGTTCTACCTGCAAATGCAAAAGAAATTGCCGGTGTTATG
TTCGTTTGGACAAATACTAATAATGAAATTATTGATGAAAATGGCCAA
ACTCTAGGAGTGAATATTGATCCACAAACATTTAAACTCTCAGGGGCA
ATGCCGGCAACTGCAATGAAAAAATTAACAGAAGCTGAAGGAGCTAAA
TTTAACACGGCAAATTTACCAGCTGCTAAGTATAAAATTTATGAAATT
CACAGTTTATCAACTTATGTCGGTGAAGATGGAGCAACCTTAACAGGT
TCTAAAGCAGTTCCAATTGAAATTGAATTACCATTGAACGATGTTGTG
GATGCGCATGTGTATCCAAAAAATACAGAAGCAAAGCCAAAAATTGAT
AAAGATTTCAAAGGTAAAGCAAATCCAGATACACCACGTGTAGATAAA
GATACACCTGTGAACCACCAAGTTGGAGATGTTGTAGAGTACGAAATT
GTTACAAAAATTCCAGCACTTGCTAATTATGCAACAGCAAACTGGAGC
GATAGAATGACTGAAGGTTTGGCATTCAACAAAGGTACAGTGAAAGTA
ACTGTTGATGATGTTGCACTTGAAGCAGGTGATTATGCTCTAACAGAA
GTAGCAACTGGTTTTGATTTGAAATTAACAGATGCTGGTTTAGCTAAA
GTGAATGACCAAAACGCTGAAAAAACTGTGAAAATCACTTATTCGGCA
ACATTGAATGACAAAGCAATTGTAGAAGTACCAGAATCTAATGATGTA
ACATTTAACTATGGTAATAATCCAGATCACGGGAATACTCCAAAGCCG
AATAAGCCAAATGAAAACGGCGATTTGACATTGACCAAGACATGGGTT
GATGCTACAGGTGCACCAATTCCGGCTGGAGCTGAAGCAACGTTCGAT
TTGGTTAATGCTCAGACTGGTAAAGTTGTACAAACTGTAACTTTGACA
ACAGACAAAAATACAGTTACTGTTAACGGATTGGATAAAAATACAGAA
TATAAATTCGTTGAACGTAGTATAAAAGGGTATTCAGCAGATTATCAA
GAAATCACTACAGCTGGAGAAATTGCTGTCAAGAACTGGAAAGACGAA
AATCCAAAACCACTTGATCCAACAGAGCCAAAAGTTGTTACATATGGT
AAAAAGTTTGTCAAAGTTAATGATAAAGATAATCGTTTAGCTGGGGCA
GAATTTGTAATTGCAAATGCTGATAATGCTGGTCAATATTTAGCACGT
AAAGCAGATAAAGTGAGTCAAGAAGAGAAGCAGTTGGTTGTTACAACA
AAGGATGCTTTAGATAGAGCAGTTGCTGCTTATAACGCTCTTACTGCA
CAACAACAAACTCAGCAAGAAAAAGAGAAAGTTGACAAAGCTCAAGCT
GCTTATAATGCTGCTGTGATTGCTGCCAACAATGCATTTGAATGGGTG
GCAGATAAGGACAATGAAAATGTTGTGAAATTAGTTTCTGATGCACAA
GGTCGCTTTGAAATTACAGGCCTTCTTGCAGGTACATATTACTTAGAA
GAAACAAAACAGCCTGCTGGTTATGCATTACTAACTAGCCGTCAGAAA
TTTGAAGTCACTGCAACTTCTTATTCAGCGACTGGACAAGGCATTGAG
TATACTGCTGGTTCAGGTAAAGATGACGCTACAAAAGTAGTCAACAAA
AAAATCACTATCCCACAAACGGGTGGTATTGGTACAATTATCTTTGCT
GTAGCGGGGGCTGCGATTATGGGTATTGCAGTGTACGCATATGTTAAA
AACAACAAAGATGAGGATCAACTTGCTTAA

An exemplary RrgB amino acid sequence (TIGR Annotation No. SP0463) is hereby provided:

(SEQ ID NO: 4)
MKSINKFLTMLAALLLTASSLFSAATVFAAGTTTTSVTVHKLLATDGD
MDKIANELETGNYAGNKVGVLPANAKEIAGVMFVWTNTNNEIIDENGQ
TLGVNIDPQTFKLSGAMPATAMKKLTEAEGAKFNTANLPAAKYKIYEI
HSLSTYVGEDGATLTGSKAVPIEIELPLNDVVDAHVYPKNTEAKPKID
KDFKGKANPDTPRVDKDTPVNHQVGDVVEYEIVTKIPALANYATANWS
DRMTEGLAFNKGTVKVTVDDVALEAGDYALTEVATGFDLKLTDAGLAK
VNDQNAEKTVKITYSATLNDKAIVEVPESNDVTFNYGNNPDHGNTPKP
NKPNENGDLTLTKTWVDATGAPIPAGAEATFDLVNAQTGKVVQTVTLT
TDKNTVTVNGLDKNTEYKFVERSIKGYSADYQEITTAGEIAVKNWKDE
NPKPLDPTEPKVVTYGKKFVKVNDKDNRLAGAEFVIANADNAGQYLAR
KADKVSQEEKQLVVTTKDALDRAVAAYNALTAQQQTQQEKEKVDKAQA
AYNAAVIAANNAFEWVADKDNENVVKLVSDAQGRFEITGLLAGTYYLE
ETKQPAGYALLTSRQKFEVTATSYSATGQGIEYTAGSGKDDATKVVNK
KITIPQTGGIGTIIFAVAGAAIMGIAVYAYVKNNKDEDQLA

RrgB contains a sortase substrate motif IPXTG (SEQ ID NO:9), shown in underscore in SEQ ID NO:4, above. A putative Cna protein B-type domain (Deivanayagam et al., 2000, Structure, 8:67-78) has been identified at amino acid residues 461-605 of SEQ ID NO:4).

An exemplary rrgC nucleic acid sequence (TIGR Annotation No. sp0464) is hereby provided:

(SEQ ID NO: 5)
ATGATTAGTCGTATCTTCTTTGTTATGGCTCTGTGTTTTTCTCTTGTA
TGGGGTGCACATGCAGTCCAAGCGCAAGAAGATCACACGTTGGTCTTG
CAATTGGAGAACTATCAGGAGGTGGTTAGTCAATTGCCATCTCGTGAT
GGTCATCGGTTGCAAGTATGGAAGTTGGATGATTCGTATTCCTATGAT
GATCGGGTGCAAATTGTAAGAGACTTGCATTCGTGGGATGAGAATAAA
CTTTCTTCTTTCAAAAAGACTTCGTTTGAGATGACCTTCCTTGAGAAT
CAGATTGAAGTATCTCATATTCCAAATGGTCTTTACTATGTTCGCTCT
ATTATCCAGACGGATGCGGTTTCTTATCCAGCTGAATTTCTTTTTGAA
ATGACAGATCAAACGGTAGAGCCTTTGGTCATTGTAGCGAAAAAAACA
GATACAATGACAACAAAGGTGAAGCTGATAAAGGTGGATCAAGACCAC
AATCGCTTGGAGGGTGTCGGCTTTAAATTGGTATCAGTAGCAAGAGAT
GTTTCTGAAAAAGAGGTTCCCTTGATTGGAGAATACCGTTACAGTTCT
TCTGGTCAAGTAGGGAGAACTCTCTATACTGATAAAAATGGAGAGATT
TTTGTGACAAATCTTCCTCTTGGGAACTATCGTTTCAAGGAGGTGGAG
CCACTGGCAGGCTATGCTGTTACGACGCTGGATACGGATGTCCAGCTG
GTAGATCATCAGCTGGTGACGATTACGGTTGTCAATCAGAAATTACCA
CGTGGCAATGTTGACTTTATGAAGGTGGATGGTCGGACCAATACCTCT
CTTCAAGGGGCAATGTTCAAAGTCATGAAAGAAGAAAGCGGACACTAT
ACTCCTGTTCTTCAAAATGGTAAGGAAGTAGTTGTAACATCAGGGAAA
GATGGTCGTTTCCGAGTGGAAGGTCTAGAGTATGGGACATACTATTTA
TGGGAGCTCCAAGCTCCAACTGGTTATGTTCAATTAACATCGCCTGTT
TCCTTTACAATCGGGAAAGATACTCGTAAGGAACTGGTAACAGTGGTT
AAAAATAACAAGCGACCACGGATTGATGTGCCAGATACAGGGGAAGAA
ACCTTGTATATCTTGATGCTTGTTGCCATTTTGTTGTTTGGTAGTGGT
TATTATCTTACGAAAAAACCAAATAACTGA

An exemplary RrgC amino acid sequence (TIGR Annotation No. SP0464) is hereby provided:

(SEQ ID NO: 6)
MISRIFFVMALCFSLVWGAHAVQAQEDHTLVLQLENYQEVVSQLPSRD
GHRLQVWKLDDSYSYDDRVQIVRDLHSWDENKLSSFKKTSFEMTFLEN
QIEVSHIPNGLYYVRSIIQTDAVSYPAEFLFEMTDQTVEPLVIVAKKT
DTMTTKVKLIKVDQDHNRLEGVGFKLVSVARDVSEKEVPLIGEYRYSS
SGQVGRTLYTDKNGEIFVTNLPLGNYRFKEVEPLAGYAVTTLDTDVQL
VDHQLVTITVVNQKLPRGNVDFMKVDGRTNTSLQGAMFKVMKEESGHY
TPVLQNGKEVVVTSGKDGRFRVEGLEYGTYYLWELQAPTGYVQLTSPV
SFTIGKDTRKELVTVVKNNKRPRIDVPDTGEETLYILMLVAILLFGSG
YYLTKKPNN

Two putative. Cna protein B-type domains (Deivanayagam et al., 2000, Structure, 8:67-78) have been identified at amino acid residues 163-251 and 273-352 of SEQ ID NO:6. RrgC contains a sortase substrate motif VPXTG (SEQ ID NO:10), shown in underscore in SEQ ID NO:6, above.

### Other Gram-Positive Bacterial Pili

The methods and compositions described herein can be used with pili from any Gram-positive bacterium. Known and putative pili proteins have been identified in GAS (e.g., Streptococcus pyogenes) (Mora et al., 2005, Proc. Natl. Acad. Sci. USA, 102:15641-6), GBS (e.g., Streptococcus agalactiae) (Lauer et al., 2005, Science, 309:105; WO 2006/078318), Actinomycetes naeslundii (Yeung et al., 1998, Infect. Immun., 66:1482-91), Corynebacterium diphtheriae (Ton-That et al., 2003, Mol. Microbiol., 50:1429-38; Ton-That and Schneewind, 2004, Trends. Microbiol., 12:228-34), Clostridium perfringens, and Enterococcus faecalis.

Pili of other Gram-positive bacteria can be used in the methods and compositions described herein. Such Gram-positive bacteria include, without limitation, firmicutes such as those of genera Streptococcus (e.g., S. pneumoniae, S. agalactiae, S. pyogenes, S. suis, S. zooepidemicus, S. viridans, S. mutans, S. gordonii, S. equi), Bacillus (e.g., B. anthracia, B. cereus, B. subtilis), Listeria (e.g., L. innocua, L. monocytogenes), Staphylococcus (e.g., S. aureus, S. epidermidis, S. caprae, S. saprophyticus, S. lugdunensis, S. schleiferi), Enterococcus (e.g., E. faecalis, E. faecium), Lactobacillus, Lactococcus (e.g., L. lactis), Leuconostoc (e.g., L. mesenteroides), Pectinatus, Pediococcus, Acetobacterium, Clostridium (e.g., C. botulinum, C. difficile, C. perfringens, C. tetani), Ruminococcus (e.g., R. albus), Heliobacterium, Heliospirillum, and Sporomusa; and actinobacteria such as those of genera Actinomycetes (e.g., A. naeslundii), Corynebacterium (e.g., C. diphtheriae, C. efficiens), Arthrobacter, Bifidobacterium (e.g., B. longum), Frankia, Micrococcus, Micromonospora, Mycobacterium (e.g., M. tuberculosis, M. leprae, M. bovis, M. africanum, M. microti), Nocardia (e.g., N. asteroides), Propionibacteriun, and Streptomyces (e.g., S. somaliensis, S. avermitilis, S. coelicolor).

### Isolated Pili

Isolated Gram-positive (e.g., S. pneumoniae) pili and other pilus-like structures that include Gram-positive pilus proteins (e.g., RrgA, RrgB, and RrgC), or fragments or variants thereof can be used in the methods described herein and as antigens in immunogenic compositions for the production of antibodies and/or the stimulation of an immune response in a subject. Pili that include variants of Gram-positive pilus proteins can also be used in the methods described herein and as antigens in immunogenic compositions for the production of antibodies and/or the stimulation of an immune response in a subject. A Gram-positive (e.g., S. pneumoniae) pilus-like polypeptide containing at least 80% sequence identity, e.g., 85%, 90%, 95%, 98%, or 99%, with a Gram-positive protein amino acid sequence (e.g., SEQ ID NO:2, 4, or 6) is also useful in the new methods. Furthermore, a Gram-positive pilus polypeptide with up to 50, e.g., 1, 3, 5, 10, 15, 20, 25, 30, or 40 amino acid insertions, deletions, or substitutions, e.g., conservative amino acid substitutions will be useful in the compositions and methods described herein.

The determination of percent identity between two amino acid sequences can be accomplished using the BLAST 2.0 program, which is available to the public at ncbi.nlm.nih.gov/BLAST. Sequence comparison is performed using an ungapped alignment and using the default parameters (BLOSUM 62 matrix, gap existence cost of 11, per residue gap cost of 1, and a lambda ratio of 0.85). The mathematical algorithm used in BLAST programs is described in Altschul et al., 1997, Nucleic Acids Research, 25:3389-3402.

As used herein, “conservative amino acid substitution” means a substitution of an amino acid in a polypeptide within an amino acid family. Families of amino acids are recognized in the art and are based on physical and chemical properties of the amino acid side chains. Families include the following: amino acids with basic side chains (e.g. lysine, arginine, and histidine); amino acids with acidic side chains (e.g., aspartic acid and glutamic acid); amino acids with uncharged polar side chains (e.g. glycine, asparagine, glutamine, serine, threonine, tyrosine, and cysteine); amino acids with nonpolar side chains (e.g. alanine, valine, leucine, isoleucine, proline, phenylalanine, methionine, and tryptophan); amino acids with branched side chains (e.g., threonine, valine, and isoleucine); and amino acids with aromatic side chains (e.g., tyrosine, phenylalanine, tryptophan, and histidine). An amino acid can belong to more than one family.

In some embodiments the immunogenic compositions of the invention comprise a Gram-positive (e.g., S. pneumoniae) pilus protein which may be formulated or purified in an oligomeric (pilus) form. In some embodiments, the oligomeric form is a hyperoligomer. In some embodiments the immunogenic compositions of the invention comprise a Gram-positive pilus protein which has been isolated in an oligomeric (pilus) form. The oligomer or hyperoligomer pilus structures comprising Gram-positive pilus proteins may be purified or otherwise formulated for use in immunogenic compositions.

One or more of the S. pneumoniae pilus protein open reading frame polynucleotide sequences may be replaced by a polynucleotide sequence coding for a fragment of the replaced ORF. Alternatively, one or more of the S. pneumoniae pilus protein open reading frames may be replaced by a sequence having sequence homology to the replaced ORF.

One or more of the Gram-positive (e.g., S. pneumoniae) pilus protein sequences typically include an LPXTG motif (such as LPXTG (SEQ ID NO:11)) or other sortase substrate motif. The LPXTG sortase substrate motif of a S. pneumoniae pilus protein may be generally represented by the formula X₁X₂X₃X₄G, wherein X at amino acid position 1 is an L, a V, an E, a Y, an 1, or a Q, wherein X at amino acid position 2 is a P if X at amino acid position 1 is an L, wherein X at amino acid position 2 is a V if X at amino acid position 1 is a E or a Q, wherein X at amino acid position 2 is a V or a P if X at amino acid position 1 is a V, wherein X at amino acid position 3 is any amino acid residue, wherein X at amino acid position 4 is a T if X at amino acid position 1 is a V, E, or Q, and wherein X at amino acid position 4 is a T, S, or A if X at amino acid position 1 is an L. Some examples of LPXTG motifs include YPXTG (SEQ ID NO:8), IPXTG (SEQ ID NO:9), LPXSG (SEQ ID NO:57), VVXTG (SEQ ID NO:12), EVXTG (SEQ ID NO:13), VPXTG (SEQ ID NO:10), QVXTG (SEQ ID NO:14), LPXAG (SEQ ID NO:15), QVPTG (SEQ ID NO:16), and FPXTG (SEQ ID NO:17).

One or more of the Gram-positive (e.g., S. pneumoniae) pilus protein sequences can include a pilin motif sequence. Some examples of pilin motif sequences include WLQDVHVYPKHQXXXXXXK (SEQ ID NO:58), WNYNVVAYPKNTXXXXXXK (SEQ ID NO:59), WLYDVNVFPKNGXXXXXXK (SEQ ID NO:60), WIYDVHVYPKNEXXXXXXK (SEQ ID NO:61), WNYNVHVYPKNTXXXXXXK (SEQ ID NO:62), FLSEINIYPKNVXXXXXXK (SEQ ID NO:63), and DVVDAHVYPKNTXXXXXXK (SEQ ID NO:64). An exemplary consensus pilin motif sequence is (W/F/E/D)-X-X-X-(V/I/A)-X-(V/I/A)-(Y/F)-P-K-(N/H/D)-XXXXXXX-(K/L) (SEQ ID NO:65) or WXXXVXVYPK (SEQ ID NO:76). The conserved internal lysine of the pilin motif can act as a nucleophile in the sortase reaction.

One or more of the Gram-positive (e.g., S. pneumoniae) pilus protein sequences can include an E-box motif sequence. Some examples of E-box motif sequences include FCLVETATASGY (SEQ ID NO:66), FCLKETKAPAGY (SEQ ID NO:67), YVLVETEAPTGF (SEQ ID NO:68), YCLVETKAPYGY (SEQ ID NO:69), YKLKETKAPYGY (SEQ ID NO:70), YPITEEVAPSGY (SEQ ID NO:71), YRLFENSEPAGY (SEQ ID NO:72), YYLWELQAPTGY (SEQ ID NO:73) and YYLEETKQPAGY (SEQ ID NO:74). An exemplary E-box motif consensus sequence is (Y/F)-X-(L/I)-X-E-T-X-(A/Q/T)-(P/A)-X-G-(Y/F) (SEQ ID NO:75) or LXET (SEQ ID NO:77).

The Gram-positive (e.g., S. pneumoniae) pili described herein can affect the ability of the Gram-positive bacteria (e.g., S. pneumoniae) to adhere to and invade epithelial cells. Pili may also affect the ability of Gram-positive bacteria (e.g., S. pneumoniae) to translocate through an epithelial cell layer. Preferably, one or more Gram-positive pili are capable of binding to or otherwise associating with an epithelial cell surface. Gram-positive pili may also be able to bind to or associate with fibrinogen, fibronectin, or collagen.

Gram-positive (e.g., S. pneumoniae) sortase proteins are thought to be involved in the secretion and anchoring of the LPXTG containing surface proteins. The S. pneumoniae sortase proteins are encoded by genes (srtB, srtC, and srtD) found in the same pathogenicity islet as the rrgA, rrgB, and rrgC genes. Sortase proteins and variants of sortase proteins useful in the methods described herein can be obtained from Gram-positive bacteria.

The Gram-positive (e.g., S. pneumoniae) pilus proteins can be covalently attached to the bacterial cell wall by membrane-associated transpeptidases, such as a sortase. The sortase may function to cleave the surface protein, preferably between the threonine and glycine residues of an LPXTG motif. The sortase may then assist in the formation of an amide link between the threonine carboxyl group and a cell wall precursor such as lipid II. The precursor can then be incorporated into the peptidoglycan via the transglycoslylation and transpeptidation reactions of bacterial wall synthesis. See Comfort et al., Infection & Immunity (2004) 72(5): 2710 - 2722.

In some embodiments, the invention includes a composition comprising oligomeric, pilus-like structures comprising a Gram-positive (e.g., S. pneumoniae) pilus protein (e.g., RrgA, RrgB, or RrgC (e.g., SEQ ID NO:2, 4, or 6)). The oligomeric, pilus-like structure may comprise numerous units of pilus protein. In some embodiments, the oligomeric, pilus-like structures comprise two or more pilus proteins. In some embodiments, the oligomeric, pilus-like structure comprises a hyper-oligomeric pilus-like structure comprising at least two (e.g., 2, 3, 4, 5, 6, 7, 8, 9, 10, 11, 12, 13, 14, 15, 20, 25, 30, 35, 40, 45, 50, 60, 70, 80, 90, 100, 120, 140, 150, 200 or more) oligomeric subunits, wherein each subunit comprises a pilus protein or a fragment thereof. The oligomeric subunits may be covalently associated via a conserved lysine within a pilin motif. The oligomeric subunits may be covalently associated via an LPXTG motif, preferably, via the threonine or serine amino acid residue, respectively. In some embodiments the oligomeric pilus-like structure is an isolated pilus.

Gram-positive (e.g., S. pneumoniae) pilus proteins or fragments thereof to be incorporated into the oligomeric, pilus-like structures of the invention will, in some embodiments, include a pilin motif.

The oligomeric pilus may be used alone or in the combinations of the invention. In some embodiments, the invention comprises a S. pneumoniae pilus in oligomeric form. In some embodiments the pilus is in a hyperoligomeric form.

### Methods of Purification of Pili

Pili can be purified from cells, such as bacterial cells, that express Gram-positive pili or pili-like structures (e.g., streptococcal pili such as pili from S. pneumoniae, group A streptococci, and group B streptococci) by separating the pili from the cells, e.g., by mechanical shearing or enzymatic digestion, and isolating the separated pili.

Suitable bacterial cells for purification of pili include piliated Gram-positive bacterial strains, non-piliated Gram-positive bacteria that have been transformed with one or more Gram-positive pilus proteins, such as S. pneumoniae RrgA, RrgB, and RrgC (e.g., SEQ ID NOs:2, 4, and 6), and Gram-negative or other cells transformed with one or more Gram-positive pilus proteins, such as S. pneumoniae RrgA, RrgB, and RrgC (e.g., SEQ ID NOs:2, 4, and 6). Typically, a cell used for purification of pili will produce only the type or types of pili desired, e.g., endogenous or heterologous pili. For the production of heterologous pili, the cell can be altered, e.g., by mutation or recombinant DNA methods, so as to not produce endogenous pili. Typically, a pili-producing Gram-positive bacterial cell useful for purification will express one or more compatible sortases such that the pili are expressed on the cell surface.

Separation of pili from Gram-positive bacterial cells is typically accomplished by mechanical shearing, enzymatic digestion, decreasing or inhibiting SrtA activity, or treatment with a compound that interferes with cell wall integrity. Mechanical shearing can physically remove the pili from the cells, whereas other methods can eliminate the point of attachment of the pili (e.g., by degradation of cell wall or pilus components). Following separation of the pili from the cells, the pili and cells can be separated, e.g., by centrifugation.

Non-limiting examples of mechanical shearing methods include ultrasonication, glass bead shearing, and mixing. Methods of sonication are discussed, for example, in Yamaguchi et al., 2004, Current Microbiol., 49:59-65. Methods of glass bead shearing are discussed, for example, in Levesque et al., 2001, J. Bacteriol., 183:2724-32. General methods of mechanical shearing are discussed, for example, in Wolfgang et al., 1998, Mol. Microbiol., 29:321-30; Trachtenberg et al., 2005, J. Mol. Biol., 346:665-676; Parge et al., 1990, J. Biol. Chem., 265:2278-85; Isaacson et al., 1981, J. Bacteriol., 146:784-9; Korhonen et al., 1980, Infect. Immun., 27:569-75; Hahn et al., 2002, J. Mol. Biol., 323:845-57; St. Geme et al., 1996, Proc. Natl. Acad. Sci. USA, 93:11913-18; Weber et al., 2005, J. Bacteriol., 187:2458-68; and Mu et al., 2002, J. Bacteriol., 184:4868-74.

Non-limiting examples of enzymes suitable for enzymatic digestion include cell-wall degrading enzymes such as mutanolysin, lysostaphin, and lysozymes. Methods of enzymatic digestion are discussed, for example, in Bender et al., 2003, J. Bacteriol., 185:6057-66; Ton-That et al., 2004, Mol. Microbiol., 53:251-61; and Ton-That et al., 2003, Mol. Microbiol., 50:1429-38. For downstream administration of pili to subjects, one can use multiple enzymes to remove cell-wall components that may cause an undesired host reaction.

Non-limiting examples of methods of inhibiting or decreasing SrtA activity include decreasing SrtA activity by introduction of a loss-of-function allele of SrtA, deleting the endogenous SrtA gene, expression of a nucleic acid that decreases SrtA expression (e.g., an antisense or miRNA), and treating the cells with a compound that inhibits SrtA activity (see, e.g., Marrafini et al., Microbiol. Mol. Biol. Rev., 70:192-221, 2006).

Exemplary sortase A inhibitors include methane-thiosulfonates (e.g., MTSET and (2-sulfonatoethyl) methane-thiosulfonate) (Ton-That and Schneewind, J. Biol. Chem., 274:24316-24320, 1999), p-hydroxymercuribenzoic acid, glucosylsterol β-sitosterol-3-O-glucopyranol (Kim et al., Biosci. Biotechnol. Biochem., 67:2477-79, 2003), berberine chloride (Kim et al., Biosci. Biotechnol. Biochem., 68:421-24, 2004), peptidyl-diazomethane (LPAT-CHN₂) (Scott et al., Biochem. J., 366:953-58, 2002), peptidyl-chloromethane (LPAT-CH₂Cl), peptidyl-vinyl sulfone [LPAT-SO₂(Ph)] (Conolly et al., J. Biol. Chem., 278:34061-65, 2003), vinyl sulfones (e.g., di-, ethyl-, methyl-, and phenyl vinyl sulfones) (Frankel et al., J. Am. Chem. Soc., 126:3404-3405, 2004), LPXTG motif peptides with the threonine residue replaced by a phosphinate group (e.g., LPEΨ{PO₂H—CH₂}G) (Kruger et al., Bioorg. Med. Chem., 12:3723-29, 2004), substituted (Z)-diaryl-acrylonitriles (Oh et al., J. Med. Chem., 47:2418-21, 2004), and extracts of various medicinal plants (Kim et al., Biosci. Biotechnol. Biochem., 66:2751-54, 2002).

Non-limiting examples of compounds that interfere with cell wall integrity include glycine and antibiotics such as penicillins (e.g., methicillin, amoxicillin, ampicillin), cephalosporins (e.g., cefalexin, cefproxil, cefepime), glycopeptides (e.g., vancomycin, teicoplanin, ramoplanin), and cycloserine.

Separated pili can be separated from other components by density, for example by using density gradient centrifugation. For example, the pili can be separated by centrifugation on a sucrose gradient.

Typically, a sample containing Gram-positive pili will contain polymers of different molecular weights due to differing numbers of pilus protein subunits present in the pili. To reduce polydispersity, a sample containing Gram-positive pili can be separated by size. For example, a gel filtration or size exclusion column can be used. An ultrafiltration membrane can also be used to reduce polydispersity of Gram-positive pili.

Gram-positive pili can also be isolated using affinity methods such as affinity chromatography. A protein that binds specifically to a Gram-positive pilus, e.g., an antibody that binds specifically to a pilus component or an antibody that binds preferentially to pili, can be immobilized on a solid substrate (e.g., a chromatography substrate) and a sample containing Gram-positive pili exposed to the immobilized binding protein. Such affinity isolation methods can also be used to isolate, purify, or enrich preparations of cells that express Gram-positive pili.

Gram-positive pili can also be isolated using any other protein purification method known in the art, e.g., precipitations, column chromatography methods, and sample concentrations. The isolating can include, e.g., gel filtration chromatography, ion-exchange chromatography, reverse phase chromatography, or affinity chromatography. Additional methods are described, e.g., in Ruffolo et al., 1997, Infect. Immun., 65:339-43. Methods of protein purification are described in detail in, e.g., Scopes, R. K., Protein Purification: Principles and Practice, 3rd. ed., 1994, Springer, N.Y.

The presence of Gram-positive pili in fractions during purification can be followed by electrophoresis (e.g., polyacrylamide electrophoresis), measuring binding of an agent that specifically binds to the gram positive pili (e.g., an antibody against a pilus protein or an antibody that binds preferentially to pili), and/or measuring an activity of the pili such as protein or cell binding.

### Antibodies

The Gram-positive (e.g., S. pneumoniae) pili of the invention may also be used to prepare antibodies specific to the Gram-positive pilus or Gram-positive pilus proteins. In some embodiments the antibodies bind specifically (e.g., preferentially) to an oligomeric or hyper-oligomeric form of a Gram-positive pilus protein. The invention also includes combinations of antibodies specific to Gram-positive pilus proteins selected to provide protection against an increased range of serotypes and strain isolates.

The Gram-positive (e.g., S. pneumoniae) pilus specific antibodies of the invention include one or more biological moieties that, through chemical or physical means, can bind to or associate with an epitope of a Gram-positive pilus polypeptide. The antibodies of the invention include antibodies that preferentially bind to a Gram-positive pilus as compared to isolated pilus proteins. The invention includes antibodies obtained from both polyclonal and monoclonal preparations, as well as the following: hybrid (chimeric) antibody molecules (see, for example, Winter et al. (1991) Nature 349: 293-299; and U.S. Pat. No. 4,816,567; F(ab′)₂ and F(ab) fragments; Fᵥ molecules (non-covalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, for example, Huston et al. (1988) Proc Natl Acad Sci USA 85:5897-5883); dimeric and trimeric antibody fragment constructs; minibodies (see, e.g., Pack et al. (1992) Biochem 31:1579-1584; Cumber et al. (1992) J Immunology 149B: 120-126); humanized antibody molecules (see, for example, Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain immunological binding properties of the parent antibody molecule. The invention further includes antibodies obtained through non-conventional processes, such as phage display.

The antibodies of the present invention can be polyclonal, monoclonal, recombinant, e.g., chimeric or humanized, fully human, non-human, e.g., murine, or single chain antibodies. Methods of making such antibodies are known. In some cases, the antibodies have effector function and can fix complement. The antibodies can also be coupled to toxins, reporter groups, or imaging agents.

In some embodiments, the Gram-positive pilus protein specific antibodies of the invention are monoclonal antibodies. Monoclonal antibodies include an antibody composition having a homogeneous antibody population. Monoclonal antibodies may be obtained from murine hybridomas, as well as human monoclonal antibodies obtained using human rather than murine hybridomas. See, e.g., Cote, et al. Monoclonal Antibodies and Cancer Therapy, Alan R. Liss, 1985, p 77.

Chimeric, humanized, e.g., completely human, antibodies are desirable for applications that include repeated administration, e.g., therapeutic treatment (and some diagnostic applications) of a human subject.

The antibodies can also be used in the prophylactic or therapeutic treatment of Gram-positive bacterial (e.g., S. pneumoniae) infection. The antibodies may block the attachment or some other activity of Gram-positive bacteria on host cells. Additionally, the antibodies can be used to deliver a toxin or therapeutic agent such as an antibiotic to Gram-positive bacterial cells.

The antibodies may be used in diagnostic applications, for example, to detect the presence or absence of Gram-positive pili or Gram-positive pilus proteins in a biological sample. Anti-pili or pilus protein antibodies can be used diagnostically to monitor protein levels in tissue as part of a clinical testing procedure, e.g., to determine the efficacy of a given treatment regimen. Detection can be facilitated by coupling (i.e., physically linking, e.g., directly or indirectly) the antibody to a detectable substance (i.e., antibody labeling). Examples of detectable substances include various enzymes, prosthetic groups, fluorescent materials, contrast agents, luminescent materials, bioluminescent materials, and radioactive materials. Examples of suitable enzymes include horseradish peroxidase, alkaline phosphatase, β-galactosidase, and acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; examples of contrast agents include electron dense materials useful for electron microscopy, such as gold particles, or magnetically active materials useful for magnetic resonance imaging, such as supermagnetic iron particles; an example of a luminescent material includes luminol; examples of bioluminescent materials include luciferase, luciferin, and aequorin, and examples of suitable radioactive material include ¹²⁵I, ¹³¹I, ³⁵S and ³H. Such diagnostic antibodies can be used in methods to detect the presence of piliated Gram-positive bacteria (e.g., S. pneumoniae) in an infected patient, e.g., by testing a sample from the patient. The course of treatment can then be selected based on the presence or absence of piliated Gram-positive bacteria. For example, a patient infected with non-piliated Gram-positive bacteria could be treated with an antibiotic, whereas a patient infected with piliated Gram-positive bacteria could also be treated with a pili-binding compound, such as an antibody, and/or an anti-inflammatory agent (e.g., IL-6 or an anti-TNF agent such as an anti-TNF antibody).

### Screening Assays

In some aspects, the invention provides methods (also referred to herein as “screening assays”) for identifying modulators, i.e., candidate compounds or agents identified from one or more test compounds (e.g., antibodies, proteins, peptides, peptidomimetics, peptoids, small inorganic molecules, small non-nucleic acid organic molecules, nucleic acids (e.g., antisense nucleic acids, siRNA, oligonucleotides, or synthetic oligonucleotides), or other drugs) that inhibit an activity, e.g., a binding activity, of Gram-positive (e.g., S. pneumoniae) pili or a Gram-positive pilus protein. Compounds thus identified can be used to modulate the activity of Gram-positive bacteria binding or attachment in a therapeutic protocol, or to elaborate the biological function of Gram-positive pili.

In some embodiments, assays are provided for screening test compounds to identify those that can bind to Gram-positive (e.g., S. pneumoniae) pili or a Gram-positive pilus protein or a portion thereof. Compounds that bind to Gram-positive pili or a Gram-positive pilus protein can be tested for their ability to modulate an activity associated with Gram-positive pili such as attachment, infection, or an inflammatory response.

The test compounds used in the methods described herein can be obtained using any of the numerous approaches in combinatorial library methods known in the art, including: biological libraries; peptoid libraries (libraries of molecules having the functionalities of peptides, but with a novel, non-peptide backbone, which are resistant to enzymatic degradation, but which, nevertheless, remain bioactive; see, e.g., Zuckermann et al., 1994, J. Med. Chem., 37:2678-2685); spatially addressable parallel solid phase or solution phase libraries; synthetic library methods requiring deconvolution; the “one-bead one-compound” library method; and synthetic library methods using affinity chromatography selection. The biological library and peptoid library approaches are limited to peptide libraries, while the other four approaches are applicable to peptide, non-peptide oligomer, or small molecule libraries of compounds (Lam, 1997, Anticancer Drug Des., 12:145).

Examples of methods for the synthesis of molecular libraries can be found in the art, for example in: DeWitt et al. (1993, Proc. Natl. Acad. Sci. USA, 90:6909; Erb et al., 1994, Proc. Natl. Acad. Sci. USA, 91:11422; Zuckermann et al., 1994, J. Med. Chem., 37:2678; Cho et al., 1993, Science, 261:1303; Carrell et al., 1994, Angew. Chem. Int. Ed. Engl., 33:2059; Carell et al., 1994, Angew. Chem. Int. Ed. Engl., 33:2061; and in Gallop et al., 1994, J. Med. Chem., 37:1233).

Libraries of compounds may be presented in solution (e.g., Houghten, 1992, Biotechniques, 13:412-421), or on beads (Lam, 1991, Nature, 354:82-84), chips (Fodor, 1993, Nature, 364:555-556), bacteria (Ladner, U.S. Patent No. 5,223,409), spores (Ladner, U.S. Pat. No. 5,223,409), plasmids (Cull et al., 1992, Proc. Natl. Acad. Sci. USA, 89:1865-1869), or on phage (Scott and Smith, 1990, Science, 249:386-390; Devlin, 1990, Science, 249:404-406; Cwirla et al., 1990, Proc. Natl. Acad. Sci. USA, 87:6378-6382; Felici, 1991, J. Mol. Biol., 222:301-310; and Ladner supra). [000113] In some embodiments, the assay is a cell-based assay in which a cell, e.g., a bacterial cell, that expresses a Gram-positive (e.g., S. pneumoniae) pili or a Gram-positive pilus protein or biologically active portion thereof is contacted with a test compound, and the ability of the test compound to modulate Gram-positive pili or a Gram-positive pilus protein activity is determined, for example, by monitoring cell binding. The cell, for example, can be of mammalian origin, e.g., murine, rat, or human origin. The cell can be an epithelial cell, e.g., an A549 lung epithelial cell.

The ability of the test compound to modulate an activity of Gram-positive (e.g., S. pneumoniae) pili or a Gram-positive pilus protein binding to a ligand or substrate, e.g., a cell or a protein such as fibrinogen, fibronectin, or collagen can be evaluated, for example, by coupling the compound, e.g., the substrate, with a radioisotope or enzymatic label such that binding of the compound, e.g., the substrate, to Gram-positive pili or a Gram-positive pilus protein can be determined by detecting the labeled compound, e.g., substrate, in a complex. Alternatively, Gram-positive pili or a Gram-positive pilus protein can be coupled with a radioisotope or enzymatic label to monitor the ability of a test compound to modulate Gram-positive pili or a Gram-positive pilus protein binding to a substrate in a complex. For example, compounds (e.g., Gram-positive pili or a Gram-positive pilus protein binding partner) can be labeled with a radioisotope (e.g., ¹²⁵I, ³⁵S, or ³H), either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound to interact with Gram-positive (e.g., S. pneumoniae) pili or a Gram-positive pilus protein with or without the labeling of any of the interactants can be evaluated. For example, a microphysiometer can be used to detect the interaction of a compound with Gram-positive pili or a Gram-positive pilus protein without labeling either the compound or the Gram-positive pili or a Gram-positive pilus protein (McConnell et al., 1992, Science 257:1906-1912). As used herein, a “microphysiometer” (e.g., Cytosensor®) is an analytical instrument that measures the rate at which a cell acidifies its environment using a light-addressable potentiometric sensor (LAPS). Changes in this acidification rate can be used as an indicator of the interaction between a compound and Gram-positive pili or a Gram-positive pilus protein.

In some embodiments, a cell-free assay is provided in which a Gram-positive (e.g., S. pneumoniae) pilus or a Gram-positive pilus protein or biologically active portion thereof is contacted with a test compound and the ability of the test compound to bind to the Gram-positive pilus or a Gram-positive pilus protein or biologically active portion thereof is evaluated. In general, biologically active portions of the Gram-positive pili or Gram-positive pilus proteins to be used in the new assays include fragments that participate in interactions with Gram-positive pili or Gram-positive pilus protein molecules.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169 and Stavrianopoulos et al., U.S. Pat. No. 4,868,103). A fluorophore label on the first ‘donor’ molecule is selected such that its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor.’ Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In some embodiments, determining the ability of a Gram-positive (e.g., S. pneumoniae) pilus or a Gram-positive (e.g., S. pneumoniae) pilus protein to bind to a target molecule (e.g., a fibrinogen, fibronectin, or collagen polypeptide or fragment thereof) can be accomplished using real-time Biomolecular Interaction Analysis (BIA) (e.g., Sjolander et al., 1991, Anal. Chem., 63:2338-2345 and Szabo et al., 1995, Curr. Opin. Struct. Biol., 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In some embodiments, the target gene product or the test substance is anchored onto a solid phase. The target gene product/test compound complexes anchored on the solid phase can be detected at the end of the reaction. The target gene product can be anchored onto a solid surface, and the test compound, which is not anchored, can be labeled, either directly or indirectly, with a detectable label discussed herein.

Multiple target gene products can be anchored onto a solid phase using protein microarray technology, which is also known by other names including: protein chip technology and solid-phase protein array technology. Protein microarray technology is well known to those of ordinary skill in the art and is based on, but not limited to, obtaining an array of identified peptides or proteins on a fixed substrate, binding target molecules or biological constituents to the peptides, and evaluating such binding. See, e.g., G. MacBeath and S. L. Schreiber, “Printing Proteins as Microarrays for High-Throughput Function Determination,” Science 289(5485):1760-1763, 2000. Microarray substrates include but are not limited to glass, silica, aluminosilicates, borosilicates, metal oxides such as alumina and nickel oxide, various clays, nitrocellulose, or nylon. The microarray substrates may be coated with a compound to enhance synthesis of a probe (e.g., a peptide) on the substrate. Coupling agents or groups on the substrate can be used to covalently link the first amino acid to the substrate. A variety of coupling agents or groups are known to those of skill in the art. Peptide probes can be synthesized directly on the substrate in a predetermined grid. Alternatively, peptide probes can be spotted on the substrate, and in such cases the substrate may be coated with a compound to enhance binding of the probe to the substrate. In these embodiments, presynthesized probes are applied to the substrate in a precise, predetermined volume and grid pattern, preferably utilizing a computer-controlled robot to apply probe to the substrate in a contact-printing manner or in a non-contact manner such as ink jet or piezo-electric delivery. Probes may be covalently linked to the substrate. In some embodiments, one or more control peptide or protein molecules are attached to the substrate. Control peptide or protein molecules allow determination of factors such as peptide or protein quality and binding characteristics, reagent quality and effectiveness, hybridization success, and analysis thresholds and success.

In some embodiments it is desirable to immobilize Gram-positive (e.g., S. pneumoniae) pili or a Gram-positive pilus protein, an anti-pilus or pilus protein antibody, or a Gram-positive pilus binding protein (e.g., an antibody) to facilitate separation of complexed from uncomplexed forms of one or both of the proteins, as well as to accommodate automation of the assay. Binding of a test compound to Gram-positive pili or a Gram-positive pilus protein, or interaction of Gram-positive pili or a Gram-positive pilus protein with a target molecule in the presence or absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, glutathione-S-transferase/pilus protein fusion proteins or glutathione-S-transferase/target fusion proteins can be adsorbed onto glutathione Sepharose™ beads (Sigma Chemical, St. Louis, Mo.) or glutathione derivatized microtiter plates, which are then combined with the test compound or the test compound and either the non-adsorbed target protein or Gram-positive pili or a Gram-positive pilus protein, and the mixture incubated under conditions conducive to complex formation (e.g., at physiological conditions for salt and pH). Following incubation, the beads or microtiter plate wells are washed to remove unbound components, the matrix immobilized in the case of beads, complex determined either directly or indirectly, for example, as described above. Alternatively, the complexes can be dissociated from the matrix, and the level of Gram-positive pili or a Gram-positive pilus protein binding or activity determined using standard techniques.

Other techniques for immobilizing either Gram-positive (e.g., S. pneumoniae) pili or a Gram-positive pilus protein or a binding target on matrices include using conjugation of biotin and streptavidin. Biotinylated Gram-positive pili or a Gram-positive pilus protein or target molecules can be prepared from biotin-NHS (N-hydroxy-succinimide) using techniques known in the art (e.g., biotinylation kits from Pierce Chemicals, Rockford, Ill.), and immobilized in the wells of streptavidin-coated 96 well plates (Pierce Chemical).

To conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In some embodiments, this assay is performed utilizing antibodies that bind specifically to Gram-positive (e.g., S. pneumoniae) pili or a Gram-positive (e.g., S. pneumoniae) pilus protein or binding targets, but do not interfere with binding of the Gram-positive pili or Gram-positive pilus protein to its target. Such antibodies can be derivatized to the wells of the plate, and unbound target or Gram-positive pili or Gram-positive pilus protein trapped in the wells by antibody conjugation. Methods for detecting such complexes, in addition to those described above for the GST-immobilized complexes, include immunodetection of complexes using antibodies reactive with the Gram-positive pili or a Gram-positive pilus protein or target molecule, as well as enzyme-linked assays which rely on detecting an enzymatic activity associated with the Gram-positive pili or a Gram-positive pilus protein or target molecule.

In some embodiments, cell-free assays can be conducted in a liquid phase. In such an assay, the reaction products are separated from unreacted components, by any of a number of standard techniques, including but not limited to: differential centrifugation (for example, Rivas et al., 1993, Trends Biochem. Sci., 18:284-287); chromatography (gel filtration chromatography, ion-exchange chromatography); electrophoresis (e.g., Ausubel et al., eds., 1999, Current Protocols in Molecular Biology, J. Wiley: New York.); and immunoprecipitation (for example, Ausubel et al., eds., 1999, Current Protocols in Molecular Biology, J. Wiley: New York). Such resins and chromatographic techniques are known to those skilled in the art (e.g., Heegaard, 1998, J. Mol. Recognit., 11:141-148 and Hage et al., 1997, J. Chromatogr. B. Biomed. Sci. Appl., 699:499-525). Further, fluorescence energy transfer may also be conveniently utilized, as described herein, to detect binding without further purification of the complex from solution.

In some embodiments, the assay includes contacting the Gram-positive (e.g., S. pneumoniae) pili or a Gram-positive (e.g., S. pneumoniae) pilus protein or biologically active portion thereof with a known cell or compound (e.g., a protein) that binds to Gram-positive pili or a Gram-positive pilus protein to form an assay mixture, contacting the assay mixture with a test compound, and determining the ability of the test compound affect binding of the Gram-positive pili or a Gram-positive pilus protein to the cell or compound.

In some embodiments an assay for binding of bacterial cells that express S. pneumoniae pili involves incubating bacterial cells that express S. pneumoniae pili with A549 lung epithelial cells, washing to remove nonadherent bacterial cells, and detecting adherent bacterial cells. Bacterial adherence can be measured by any means in the art, e.g., detecting binding of an antibody to the adherent bacterial cells or lysing the epithelial cells and counting the number of associated bacterial cells. HEP2 cells, CHO cells, or HeLa cells can also be used in assays of binding of bacterial cells that express S. pneumoniae pili.

### Immunogenic Compositions

Immunogenic compositions of the invention that include Gram-positive (e.g., S. pneumoniae) pili may further comprise one or more antigenic agents. Exemplary antigens include those listed below. Additionally, the compositions of the present invention may be used to treat or prevent infections caused by any of the below-listed microbes or related microbes. Antigens for use in the immunogenic compositions include, but are not limited to, one or more of the following set forth below, or antigens derived from one or more of the following set forth below:

Bacterial Antigens

N. meningitides: a protein antigen from N. meningitides serogroup A, C, W135, Y, and/or B (1-7); an outer-membrane vesicle (OMV) preparation from N. meningitides serogroup B. (8, 9, 10, 11); a saccharide antigen, including LPS, from N. meningitides serogroup A, B, C W135 and/or Y, such as the oligosaccharide from serogroup C (see PCT/US99/09346; PCT IB98/01665; and PCT IB99/00103);

Streptococcus pneumoniae: a saccharide or protein antigen, particularly a saccharide from Streptococcus pneumoniae or a protein or antigenic peptide of PhtD (BVH-11-2, SP1003, spr0907) (Adamou et al., Infect. Immun., 69:949-53, 2001; Hamel et al., Infect. Immun., 72:2659-70, 2004); PhtE (BVH-3, SP1004, spr0908) (Adamou et al., Infect. Immun., 69:949-53, 2001; Hamel et al., Infect. Immun., 72:2659-70, 2004); PhtB (PhpA, BVH-11, SP1174, spr1060) (Adamou et al., Infect. Immun., 69:949-53, 2001; Zhang et al., Infect. Immun., 69:3827-36, 2001; Hamel et al., Infect. Immun., 72:2659-70, 2004); PhtA (BVH-11-3, SP1175, spr1061) (Adamou et al., Infect. Immun., 69:949-53, 2001; Wizemann et al., Infect. Immun., 69:1593-98, 2001; Zhang et al., Infect. Immun., 69:3827-36, 2001; Hamel et al., Infect. Immun., 72:2659-70, 2004); NanA (SP1693, spr1536) (Tong et al., Infect. Immun., 73:7775-78, 2005); SP1872 (spr1687) (Brown et al., Infect. Immun., 69:6702-06, 2001); PspC (CbpA, SP2190, spr1995) (Ogunniyi et al., Infect. Immun., 69:5997-6003, 2001); PspA (SP0177, spr0121, spr1274) (Briles et al., Vaccine, 19:S87-S95, 2001); SP0498 (spr0440); LytB (SP0965, spr0867) (Wizemann et al., Infect. Immun., 69:1593-98, 2001); AIiB (SP1527, spr1382); PpmA (SP0981, spr0884) (Overweg et al., Infect. Immun., 68:4180-4188, 2000); LytC (SP1573, spr1431) (Wizemann et al., Infect. Immun., 69:1593-98, 2001); PsaA (Briles et al., Vaccine, 19:S87-S95, 2001); PdB (Ogunniyi et al., Infect. Immun., 69:5997-6003, 2001); RPhp (Zhang et al., Infect. Immun., 69:3827-36, 2001); PiuA (Jomaa et al., Vaccine, 24:5133-39, 2006); PiaA (Jomaa et al., Vaccine, 24:5133-39, 2006); 6PGD (Daniely et al., Clin. Exp. Immunol., 144:254-263, 2006); or PppA (Green et al., Infect. Immun., 73:981-89, 2005);

Streptococcus agalactiae: particularly, Group B streptococcus antigens;

Streptococcus pyogenes: particularly, Group A streptococcus antigens;

Enterococcus faecalis or Enterococcus faecium: Particularly a trisaccharide repeat or other Enterococcus derived antigens provided in U.S. Pat. No. 6,756,361;

Helicobacter pylori: including: Cag, Vac, Nap, HopX, HopY and/or urease antigen;

Bordetella pertussis: such as pertussis holotoxin (PT) and filamentous hemagglutinin (FHA) from B. pertussis, optionally also combination with pertactin and/or agglutinogens 2 and 3 antigen;

Staphylococcus aureus: including S. aureus type 5 and 8 capsular polysaccharides optionally conjugated to nontoxic recombinant Pseudomonas aeruginosa exotoxin A, such as StaphVAX™, or antigens derived from surface proteins, invasins (leukocidin, kinases, hyaluronidase), surface factors that inhibit phagocytic engulfment (capsule, Protein A), carotenoids, catalase production, Protein A, coagulase, clotting factor, and/or membrane-damaging toxins (optionally detoxified) that lyse eukaryotic cell membranes (hemolysins, leukotoxin, leukocidin);

Staphylococcus epidermis: particularly, S. epidermidis slime-associated antigen (SAA);

Staphylococcus saprophyticus: (causing urinary tract infections) particularly the 160 kDa hemagglutinin of S. saprophyticus antigen;

Pseudomonas aeruginosa: particularly, endotoxin A, Wzz protein, P. aeruginosa LPS, more particularly LPS isolated from PAO1 (O5 serotype), and/or Outer Membrane Proteins, including Outer Membrane Proteins F (OprF) (Infect Immun. 2001 May; 69(5): 3510-3515);

Bacillus anthracis (anthrax): such as B. anthracis antigens (optionally detoxified) from A-components (lethal factor (LF) and edema factor (EF)), both of which can share a common B-component known as protective antigen (PA);

Moraxella catarrhalis: (respiratory) including outer membrane protein antigens (HMW-OMP), C-antigen, and/or LPS;

Yersinia pestis (plague): such as F1 capsular antigen (Infect Immun. 2003 January; 71(1)): 374-383, LPS (Infect Immun. 1999 October; 67(10): 5395), Yersinia pestis V antigen (Infect Immun. 1997 November; 65(11): 4476-4482);

Yersinia enterocolitica (gastrointestinal pathogen): particularly LPS (Infect Immun. 2002 August; 70(8): 4414);

Yersinia pseudotuberculosis: gastrointestinal pathogen antigens;

Mycobacterium tuberculosis: such as lipoproteins, LPS, BCG antigens, a fusion protein of antigen 85B (Ag85B) and/or ESAT-6 optionally formulated in cationic lipid vesicles (Infect Immun. 2004 October; 72(10): 6148), Mycobacterium tuberculosis (Mtb) isocitrate dehydrogenase associated antigens (Proc Natl Acad Sci USA. 2004 August 24; 101(34): 12652), and/or MPT51 antigens (Infect Immun. 2004 July; 72(7): 3829);

Legionella pneumophila (Legionnaires' Disease): L. pneumophila antigens—optionally derived from cell lines with disrupted asd genes (Infect Immun. 1998 May; 66(5): 1898);

Rickettsia: including outer membrane proteins, including the outer membrane protein A and/or B (OmpB) (Biochim Biophys Acta. 2004 Nov. 1; 1702(2):145), LPS, and surface protein antigen (SPA) (J Autoimmun. 1989 June; 2 Supp1:81);

E. coli: including antigens from enterotoxigenic E. coli (ETEC), enteroaggregative E. coli (EAggEC), diffusely adhering E. coli (DAEC), enteropathogenic E. coli (EPEC), and/or enterohemorrhagic E. coli (EHEC);

Vibrio cholerae: including proteinase antigens, LPS, particularly lipopolysaccharides of Vibrio cholerae II, O1 Inaba O-specific polysaccharides, V. cholera O139, antigens of IEM108 vaccine (Infect Immun. 2003 Oct;71(10):5498-504), and/or Zonula occludens toxin (Zot);

Salmonella typhi (typhoid fever): including capsular polysaccharides preferably conjugates (Vi, i.e. vax-TyVi);

Salmonella typhimurium (gastroenteritis): antigens derived therefrom are contemplated for microbial and cancer therapies, including angiogenesis inhibition and modulation of flk;

Listeria monocytogenes (systemic infections in immunocompromised or elderly people, infections of fetus): antigens derived from L. monocytogenes are preferably used as carriers/vectors for intracytoplasmic delivery of conjugates/associated compositions of the present invention;

Porphyromonas gingivalis: particularly, P. gingivalis outer membrane protein (OMP);

Tetanus: such as tetanus toxoid (TT) antigens, preferably used as a carrier protein in conjunction/conjugated with the compositions of the present invention;

Diphtheria: such as a diphtheria toxoid, (e.g., CRM₁₉₇), additionally antigens capable of modulating, inhibiting or associated with ADP ribosylation are contemplated for combination/co-administration/conjugation with the compositions of the present invention, the diphtheria toxoids can be used as carrier proteins;

Borrelia burgdorferi (Lyme disease): such as antigens associated with P39 and P13 (an integral membrane protein, Infect Immun. 2001 May; 69(5): 3323-3334), VlsE Antigenic Variation Protein (J. Clin. Microbiol. 1999 December; 37(12): 3997);

Haemophilus influenzae B: such as a saccharide antigen therefrom;

Klebsiella: such as an OMP, including OMP A, or a polysaccharide optionally conjugated to tetanus toxoid;

Neiserria gonorrhoeae: including, a Por (or porin) protein, such as PorB (see Zhu et al., Vaccine (2004) 22:660-669), a transferring binding protein, such as TbpA and TbpB (See Price et al., Infection and Immunity (2004) 71(1):277-283), a opacity protein (such as Opa), a reduction-modifiable protein (Rmp), and outer membrane vesicle (OMV) preparations (see Plante et al., J Infectious Disease (2000) 182:848-855), also see e.g. WO99/24578, WO99/36544, WO99/57280, WO02/079243);

Chlamydia pneumoniae: particularly C. pneumoniae protein antigens;

Chlamydia trachomatis: including antigens derived from serotypes A, B, Ba and C are (agents of trachoma, a cause of blindness), serotypes L₁, L₂ & L₃ (associated with Lymphogranuloma venereum), and serotypes, D-K;

Treponema pallidum (Syphilis): particularly a TmpA antigen; and

Haemophilus ducreyi (causing chancroid): including outer membrane protein (DsrA).

Where not specifically referenced, further bacterial antigens of the invention may be capsular antigens, polysaccharide antigens or protein antigens of any of the above. Further bacterial antigens may also include an outer membrane vesicle (OMV) preparation. Additionally, antigens include live, attenuated, and/or purified versions of any of the aforementioned bacteria. The bacterial or microbial derived antigens of the present invention may be gram-negative or gram-positive and aerobic or anaerobic.

Additionally, any of the above bacterial-derived saccharides (polysaccharides, LPS, LOS or oligosaccharides) can be conjugated to another agent or antigen, such as a carrier protein (for example CRM₁₉₇). Such conjugation may be direct conjugation effected by reductive amination of carbonyl moieties on the saccharide to amino groups on the protein, as provided in U.S. Pat. No. 5,360,897 and Can J Biochem Cell Biol. 1984 May; 62(5):270-5. Alternatively, the saccharides can be conjugated through a linker, such as, with succinamide or other linkages provided in Bioconjugate Techniques, 1996 and CRC, Chemistry of Protein Conjugation and Cross-Linking, 1993.

Viral Antigens

Influenza: including whole viral particles (attenuated), split, or subunit comprising hemagglutinin (HA) and/or neuraminidase (NA) surface proteins, the influenza antigens may be derived from chicken embryos or propagated on cell culture, and/or the influenza antigens are derived from influenza type A, B, and/or C, among others;

Respiratory syncytial virus (RSV): including the F protein of the A2 strain of RSV (J Gen Virol. 2004 November; 85(Pt 11):3229) and/or G glycoprotein;

Parainfluenza virus (PIV): including PIV type 1, 2, and 3, preferably containing hemagglutinin, neuraminidase and/or fusion glycoproteins;

Poliovirus: including antigens from a family of picornaviridae, preferably poliovirus antigens such as OPV or, preferably IPV;

Measles: including split measles virus (MV) antigen optionally combined with the Protollin and or antigens present in MMR vaccine;

Mumps: including antigens present in MMR vaccine;

Rubella: including antigens present in MMR vaccine as well as other antigens from Togaviridae, including dengue virus;

Rabies: such as lyophilized inactivated virus (RabAvert™);

Flaviviridae viruses: such as (and antigens derived therefrom) yellow fever virus, Japanese encephalitis virus, dengue virus (types 1, 2, 3, or 4), tick borne encephalitis virus, and West Nile virus;

Caliciviridae; antigens therefrom;

HIV: including HIV-1 or HIV-2 strain antigens, such as gag (p24gag and p55gag), env (gp160 and gp41), pol, tat, nef, rev vpu, miniproteins, (preferably p55 gag and gp140v delete) and antigens from the isolates HIV_{IIIb}, HIV_{SF2}, HIV_{LAV}, HIV_{LA1}, HIV_{MN}, HIV-1_{CM235}, HIV-1_{US4}, HIV-2; simian immunodeficiency virus (SIV) among others;

Rotavirus: including VP4, VPS, VP6, VP7, VP8 proteins (Protein Expr Purif. 2004 December; 38(2):205) and/or NSP4;

Pestivirus: such as antigens from classical porcine fever virus, bovine viral diarrhea virus, and/or border disease virus;

Parvovirus: such as parvovirus B19;

Coronavirus: including SARS virus antigens, particularly spike protein or proteases therefrom, as well as antigens included in WO 04/92360;

Hepatitis A virus: such as inactivated virus; Hepatitis B virus: such as the surface and/or core antigens (sAg), as well as the presurface sequences, pre-S1 and pre-S2 (formerly called pre-S), as well as combinations of the above, such as sAg/pre-S1, sAg/pre-S2, sAg/pre-S1/pre-S2, and pre-S1/pre-S2, (see, e.g., AHBV Vaccines—Human Vaccines and Vaccination, pp. 159-176; and U.S. Pat. Nos. 4,722,840, 5,098,704, 5,324,513; Beames et al., J. Virol. (1995) 69:6833-6838, Birnbaum et al., J. Virol. (1990) 64:3319-3330; and Zhou et al., J. Virol. (1991) 65:5457-5464);

Hepatitis C virus: such as E1, E2, E1/E2 (see, Houghton et al., Hepatology (1991) 14:381), NS345 polyprotein, NS 345-core polyprotein, core, and/or peptides from the nonstructural regions (International Publication Nos. WO 89/04669; WO 90/11089; and WO 90/14436);

Delta hepatitis virus (HDV): antigens derived therefrom, particularly δ-antigen from HDV (see, e.g., U.S. Pat. No. 5,378,814);

Hepatitis E virus (HEV); antigens derived therefrom;

Hepatitis G virus (HGV); antigens derived therefrom;

Varcicella zoster virus: antigens derived from varicella zoster virus (VZV) (J. Gen. Virol. (1986) 67:1759);

Epstein-Barr virus: antigens derived from EBV (Baer et al., Nature (1984) 310:207);

Cytomegalovirus: CMV antigens, including gB and gH (Cytomegaloviruses (J. K. McDougall, ed., Springer-Verlag 1990) pp. 125-169);

Herpes simplex virus: including antigens from HSV-1 or HSV-2 strains and glycoproteins gB, gD and gH (McGeoch et al., J. Gen. Virol. (1988) 69:1531 and U.S. Pat. No. 5,171,568);

Human Herpes Virus: antigens derived from other human herpesviruses such as HHV6 and HHV7; and

HPV: including antigens associated with or derived from human papillomavirus (HPV), for example, one or more of E1-E7, L1, L2, and fusions thereof, particularly the compositions of the invention may include a virus-like particle (VLP) comprising the L1 major capsid protein, more particular still, the HPV antigens are protective against one or more of HPV serotypes 6, 11, 16 and/or 18.

Further provided are antigens, compositions, methods, and microbes included in Vaccines, 4^{th} Edition (Plotkin and Orenstein ed. 2004); Medical Microbiology 4^{th} Edition (Murray et al. ed. 2002); Virology, 3rd Edition (W. K. Joklik ed. 1988); Fundamental Virology, 2nd Edition (B. N. Fields and D. M. Knipe, eds. 1991), which are contemplated in conjunction with the compositions of the present invention.

Additionally, antigens include live, attenuated, split, and/or purified versions of any of the aforementioned viruses.

Fungal Antigens

Fungal antigens for use herein, associated with vaccines include those described in: U.S. Pat. Nos. 4,229,434 and 4,368,191 for prophylaxis and treatment of trichopytosis caused by Trichophyton mentagrophytes; U.S. Pat. Nos. 5,277,904 and 5,284,652 for a broad spectrum dermatophyte vaccine for the prophylaxis of dermatophyte infection in animals, such as guinea pigs, cats, rabbits, horses and lambs, these antigens comprises a suspension of killed T. equinum, T. mentagrophytes (var. granulare), M. canis and/or M. gypseum in an effective amount optionally combined with an adjuvant; U.S. Pat. Nos. 5,453,273 and 6,132,733 for a ringworm vaccine comprising an effective amount of homogenized, formaldehyde-killed fungi, i.e., Microsporum canis culture in a carrier; U.S. Pat. No. 5,948,413 involving extracellular and intracellular proteins for pythiosis. Additional antigens identified within antifungal vaccines include Ringvac bovis LTF-130 and Bioveta. [000137] Further, fungal antigens for use herein may be derived from Dermatophytres, including: Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T. verrucosum var. album, var. discoides, var. ochraceum, Trichophyton violaceum, and/or Trichophyton faviforme.

Fungal pathogens for use as antigens or in derivation of antigens in conjunction with the compositions of the present invention comprise Aspergillus fumigatus, Aspergillus flavus, Aspergillus niger, Aspergillus nidulans, Aspergillus terreus, Aspergillus sydowi, Aspergillus flavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, and Saksenaea spp.

Other fungi from which antigens can be derived include Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp.

Processes for producing fungal antigens are well known in the art (see U.S. Pat. No. 6,333,164). In some embodiments a solubilized fraction is extracted and separated from an insoluble fraction obtainable from fungal cells of which cell wall has been substantially removed or at least partially removed, characterized in that the process comprises obtaining living fungal cells; obtaining fungal cells of which cell wall has been substantially removed or at least partially removed; bursting the fungal cells of which cell wall has been substantially removed or at least partially removed; obtaining an insoluble fraction; and extracting and separating a solubilized fraction from the insoluble fraction.

STD Antigens

In some embodiments, microbes (bacteria, viruses and/or fungi) against which the present compositions and methods can be implemented include those that cause sexually transmitted diseases (STDs) and/or those that display on their surface an antigen that can be the target or antigen composition of the invention. In some embodiments of the invention, compositions are combined with antigens derived from a viral or bacterial STD. Antigens derived from bacteria or viruses can be administered in conjunction with the compositions of the present invention to provide protection against at least one of the following STDs, among others: chlamydia, genital herpes, hepatitis (particularly HCV), genital warts, gonorrhea, syphilis and/or chancroid (see, e.g., WO 00/15255).

In some embodiments, the compositions of the present invention are co-administered with an antigen for the prevention or treatment of an STD.

Antigens derived from the following viruses associated with STDs, which are described in greater detail above, are co-administered with the compositions of the present invention: hepatitis (particularly HCV), HPV, HIV, or HSV.

Additionally, antigens derived from the following bacteria associated with STDs, which are described in greater detail above, are co-administered with the compositions of the present invention: Neiserria gonorrhoeae, Chlamydia pneumoniae, Chlamydia trachomatis, Treponema pallidum, or Haemophilus ducreyi.

Respiratory Antigens

In some embodiments the Gram positive (e.g., S. pneumoniae) pilus antigen is a respiratory antigen and is used in an immunogenic composition for methods of preventing and/or treating infection by a respiratory pathogen, including a virus, bacteria, or fungi such as respiratory syncytial virus (RSV), PIV, SARS virus, influenza, Bacillus anthracis, particularly by reducing or preventing infection and/or one or more symptoms of respiratory virus infection. A composition comprising an antigen described herein, such as one derived from a respiratory virus, bacteria or fungus is administered in conjunction with the compositions of the present invention to an individual at risk of being exposed to that particular respiratory microbe, has been exposed to a respiratory microbe or is infected with a respiratory virus, bacteria or fungus. The composition(s) of the present invention can be co-administered at the same time or in the same formulation with an antigen of the respiratory pathogen. Administration of the composition results in reduced incidence and/or severity of one or more symptoms of respiratory infection.

Pediatric/Geriatric Antigens

In some embodiments the compositions of the present invention are used in conjunction with one or more antigens for treatment of a pediatric population, as in a pediatric antigen. In some embodiments the age of subjects in the pediatric population is less than about 3 years old, or less than about 2 years, or less than about 1 years old. In some embodiments the pediatric antigen (in conjunction with the composition of the present invention) is administered multiple times over at least 1, 2, or 3 years.

In some embodiment the compositions of the present invention are used in conjunction with one or more antigens for treatment of a geriatric population, as in a geriatric antigen. In some embodiments, the age of subjects in the geriatric population is greater than 50, 55, 60, 65, 70 or 75 years old.

Other Antigens

Other antigens for use in conjunction with the compositions of the present include hospital acquired (nosocomial) associated antigens.

In some embodiments, parasitic antigens are contemplated in conjunction with the compositions of the present invention. Examples of parasitic antigens include those derived from organisms causing diseases including but not limited to malaria and/or Lyme disease.

In some embodiments, the antigens in conjunction with the compositions of the present invention are associated with and/or effective against a mosquito born illness. In some embodiments, the antigens in conjunction with the compositions of the present invention are associated with and/or effective against encephalitis. In some embodiments the antigens in conjunction with the compositions of the present invention are associated with and/or effective against an infection of the nervous system.

In some embodiments, the antigens in conjunction with the compositions of the present invention are antigens transmissible through blood or body fluids.

Antigen Formulations

In some aspects of the invention, methods of producing microparticles having adsorbed antigens are provided. The methods comprise: (a) providing an emulsion by dispersing a mixture comprising (i) water, (ii) a detergent, (iii) an organic solvent, and (iv) a biodegradable polymer selected from the group consisting of a poly(a-hydroxy acid), a polyhydroxy butyric acid, a polycaprolactone, a polyorthoester, a polyanhydride, and a polycyanoacrylate. The polymer is typically present in the mixture at a concentration of about 1% to about 30% relative to the organic solvent, while the detergent is typically present in the mixture at a weight-to-weight detergent-to-polymer ratio of from about 0.00001:1 to about 0.1:1 (more typically about 0.0001:1 to about 0.1:1, about 0.001:1 to about 0.1:1, or about 0.005:1 to about 0.1:1); (b) removing the organic solvent from the emulsion; and (c) adsorbing an antigen on the surface of the microparticles. In some embodiments, the biodegradable polymer is present at a concentration of about 3% to about 10% relative to the organic solvent.

In some embodiments microparticles for use herein can be formed from materials that are sterilizable, non-toxic and biodegradable. Such materials include, without limitation, poly(α-hydroxy acid), polyhydroxybutyric acid, polycaprolactone, polyorthoester, polyanhydride, PACA, and polycyanoacrylate. In some embodiments, microparticles for use with the present invention are derived from a poly(α-hydroxy acid), in particular, from a poly(lactide) (“PLA”) or a copolymer of D,L-lactide and glycolide or glycolic acid, such as a poly(D,L-lactide-co-glycolide) (“PLG” or “PLGA”), or a copolymer of D,L-lactide and caprolactone. The microparticles may be derived from any of various polymeric starting materials which have a variety of molecular weights and, in the case of the copolymers such as PLG, a variety of lactide:glycolide ratios, the selection of which will be largely a matter of choice, depending in part on the coadministered macromolecule. These parameters are discussed more fully below.

Further antigens may also include an outer membrane vesicle (OMV) preparation.

Antigens can also be adsorbed to peptidoglycans of various gram-positive bacteria to make gram-positive enhancer matrix (GEM) particles, as described in Bosma et al., Appl. Env. Microbiol., 72:880-889, 2006, the entire contents of which are incorporated herein by reference. This method relies on the non-covalent binding of the LysM motif (Buist et al., J. Bact., 177:1554-63, 1995; Bateman and Bycroft, J. Mol. Biol., 299:1113-19, 2000) to the cell wall peptidoglycan of acid-treated cells. Briefly, a polypeptide antigen linked to one or more LysM motifs (e.g., non-covalently or covalently (e.g., as a fusion protein or by conjugation) is added to acid-treated gram-positive bacteria. The antigen peptides bind with high affinity and can be used in immunogenic compositions. Exemplary acids used in these methods include trichloroacetic acid (e.g., at 0.1%-10%), acetic acid (e.g., at 5.6 M), HCl (e.g., at 0.01 M), lactic acid (e.g., at 0.72 M), and formic acid (e.g., at 0.56 M).

Additional formulation methods and antigens (especially tumor antigens) are provided in U.S. patent Ser. No. 09/581,772.

Antigen References

The following references, each of which is specifically incorporated by reference in its entirety, include antigens useful in conjunction with the compositions of the present invention:
International patent application WO99/24578
International patent application WO99/36544.
International patent application WO99/57280.
International patent application WO00/22430.
Tettelin et al. (2000) Science 287:1809-1815.
International patent application WO96/29412.
Pizza et al. (2000) Science 287:1816-1820.
PCT WO 01/52885.
Bjune et al. (1991) Lancet 338(8775).
Fuskasawa et al. (1999) Vaccine 17:2951-2958.
Rosenqist et al. (1998) Dev. Biol. Strand 92:323-333.
Costantino et al. (1992) Vaccine 10:691-698.
Costantino et al. (1999) Vaccine 17:1251-1263.
Watson (2000) Pediatr Infect Dis J 19:331-332.
Rubin (20000) Pediatr Clin North Am 47:269-285,v.
Jedrzejas (2001) Microbiol Mol Biol Rev 65:187-207.
International patent application filed on 3 Jul. 2001 claiming priority from GB0016363.4;WO 02/02606; PCT IB/01/00166.
Kalman et al. (1999) Nature Genetics 21:385-389.
Read et al. (2000) Nucleic Acids Res 28:1397-406.
Shirai et al. (2000) J. Infect. Dis 181(Suppl 3):S524-S527.
International patent application WO99/27105.
International patent application WO00/27994.
International patent application WO00/37494.
International patent application WO99/28475.
Bell (2000) Pediatr Infect Dis J 19:1187-1188.
Iwarson (1995) APMIS 103:321-326.
Gerlich et al. (1990) Vaccine 8 Suppl:S63-68 & 79-80.
Hsu et al. (1999) Clin Liver Dis 3:901-915.
Gastofsson et al. (1996) N. Engl. J. Med. 334-:349-355.
Rappuoli et al. (1991) TIBTECH 9:232-238.
Vaccines (1988) eds. Plotkin & Mortimer. ISBN 0-7216-1946-0.
Del Guidice et al. (1998) Molecular Aspects of Medicine 19:1-70.
International patent application WO93/018150.
International patent application WO99/53310.
International patent application WO98/04702.
Ross et al. (2001) Vaccine 19:135-142.
Sutter et al. (2000) Pediatr Clin North Am 47:287-308.
Zimmerman & Spann (1999) Am Fan Physician 59:113-118, 125-126.
Dreensen (1997) Vaccine 15 Suppl″S2-6.
MMWR Morb Mortal Wkly rep Jan. 16, 1998: 47(1):12, 9.
McMichael (2000) Vaccine 19 Suppl 1:S101-107.
Schuchat (1999) Lancet 353(9146):51-6.
GB patent applications 0026333.5, 0028727.6 & 0105640.7.
Dale (1999) Infect Disclin North Am 13:227-43, viii.
Ferretti et al. (2001) PNAS USA 98: 4658-4663.
Kuroda et al. (2001) Lancet 357(9264):1225-1240; see also pages 1218-1219.
Ramsay et al. (2001) Lancet 357(9251):195-196.
Lindberg (1999) Vaccine 17 Suppl 2:S28-36.
Buttery & Moxon (2000) J R Coil Physicians Long 34:163-168.
Ahmad & Chapnick (1999) Infect Dis Clin North Am 13:113-133, vii.
Goldblatt (1998) J. Med. Microbiol. 47:663-567.
European patent 0 477 508.
U.S. Pat. No. 5,306,492.
International patent application WO98/42721.
Conjugate Vaccines (eds. Cruse et al.) ISBN 3805549326, particularly vol. 10:48-114.
Hermanson (1996) Bioconjugate Techniques ISBN: 012323368 & 012342335X.
European patent application 0372501.
European patent application 0378881.
European patent application 0427347.
International patent application WO93/17712.
International patent application WO98/58668.
European patent application 0471177.
International patent application WO00/56360.
International patent application WO00/67161.

Fusion Proteins

The Gram-positive (e.g., S. pneumoniae) proteins used in the invention may be present in the composition as individual separate polypeptides. In some embodiments at least two (i.e. 2, 3, 4, 5, 6, 7, 8, 9, 10, 11, 12, 13, 14, 15, 16, 17, or 18) of the antigens are expressed as a single polypeptide chain (a “hybrid” or “fusion” polypeptide) that includes a pilus subunit. Such fusion polypeptides offer two principal advantages: first, a polypeptide that may be unstable or poorly expressed on its own can be assisted by adding a suitable fusion partner that overcomes the problem; second, commercial manufacture is simplified as only one expression and purification need be employed in order to produce two polypeptides which are both antigenically useful.

The fusion polypeptide may comprise one or more Gram-positive (e.g., S. pneumoniae) pilus polypeptide sequences. Accordingly, the invention includes one or more fusion peptides comprising a first amino acid sequence and a second amino acid sequence, wherein said first and second amino acid sequences are selected from a Gram-positive pilus protein or a fragment thereof. In some embodiments, the first and second amino acid sequences in the fusion polypeptide comprise different epitopes of the same protein.

In some embodiments the present invention provides hybrids (or fusions) comprising amino acid sequences from two, three, four, five, six, seven, eight, nine, or ten antigens. In some embodiments, the invention provides hybrids comprising amino acid sequences from two, three, four, or five antigens.

Different hybrid polypeptides may be mixed together in a single formulation. Within such combinations, a Gram-positive (e.g., S. pneumoniae) pilus sequence may be present in more than one hybrid polypeptide and/or as a non-hybrid polypeptide. In some embodiments an antigen is present either as a hybrid or as a non-hybrid, but not as both.

Hybrid polypeptides can be represented by the formula NH₂-A-{-X-L-}ₙ-B—COOH, wherein: X is an amino acid sequence of a Gram-positive (e.g., S. pneumoniae) pilus protein or a fragment thereof; L is an optional linker amino acid sequence; A is an optional N-terminal amino acid sequence; B is an optional C-terminal amino acid sequence; and n is 2, 3, 4, 5, 6, 7, 8, 9, 10, 11, 12, 13, 14 or 15.

If a —X— moiety has a leader peptide sequence in its wild-type form, this may be included or omitted in the hybrid protein. In some embodiments, the leader peptides are deleted except for that of the —X— moiety located at the N-terminus of the hybrid protein i.e. the leader peptide of X₁ will be retained, but the leader peptides of X₂ . . . Xₙ will be omitted. This is equivalent to deleting all leader peptides and using the leader peptide of X₁ as moiety -A-.

For each n instances of {—X-L-}, linker amino acid sequence -L- may be present or absent. For instance, when n=2 the hybrid may be NH₂—X₁-L₁-X₂-L₂-COOH, NH₂—X₁—X₂—COOH, NH₂—X₁-L₁-X₂—COOH, NH₂—X₁—X₂-L₂-COOH, etc. Linker amino acid sequence(s) -L- will typically be short (e.g., 20 or fewer amino acids, i.e., 19, 18, 17, 16, 15, 14, 13, 12, 11, 10, 9, 8, 7, 6, 5, 4, 3, 2, 1). Examples comprise short peptide sequences which facilitate cloning, poly-glycine linkers (i.e., comprising Glyₙ where n=2, 3, 4, 5, 6, 7, 8, 9, 10 or more), and histidine tags (i.e., Hisₙ where n=3, 4, 5, 6, 7, 8, 9, 10 or more). Other suitable linker amino acid sequences will be apparent to those skilled in the art. A useful linker is GSGGGG (SEQ ID NO:53), with the Gly-Ser dipeptide being formed from a BamHI restriction site, thus aiding cloning and manipulation, and the (Gly)₄ tetrapeptide being a typical poly-glycine linker.

In some embodiments -A- is an optional N-terminal amino acid sequence. This will typically be short (e.g. 40 or fewer amino acids, i.e., 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct protein trafficking, or short peptide sequences which facilitate cloning or purification (e.g., histidine tags, i.e., His where n=3, 4, 5, 6, 7, 8, 9, 10 or more). Other suitable N-terminal amino acid sequences will be apparent to those skilled in the art. If X₁ lacks its own N-terminus methionine, in some embodiments -A- is an oligopeptide (e.g., with 1, 2, 3, 4, 5, 6, 7 or 8 amino acids) which provides a N-terminus methionine.

In some embodiments —B— is an optional C-terminal amino acid sequence. This will typically be short (e.g. 40 or fewer amino acids, i.e., 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short peptide sequences which facilitate cloning or purification (e.g., comprising histidine tags, i.e., His where n=3, 4, 5, 6, 7, 8, 9, 10 or more), or sequences which enhance protein stability. Other suitable C-terminal amino acid sequences will be apparent to those skilled in the art.

In some embodiments, n is 2 or 3.

### Immunogenic Compositions and Medicaments

In some embodiments compositions of the invention are immunogenic compositions. In some embodiments the compositions are vaccine compositions. In some embodiments the pH of the composition is between 6 and 8, and, in some embodiments, is about 7. The pH may be maintained by the use of a buffer. The composition may be sterile and/or pyrogen-free. The composition may be isotonic with respect to humans. In some embodiments the composition is a sterile injectable.

Vaccines according to the invention may either be prophylactic (i.e. to prevent infection) or therapeutic (i.e. to treat infection). Accordingly, the invention provides methods for the therapeutic or prophylactic treatment of a Gram-positive bacterial (e.g., S. pneumoniae) infection in an animal susceptible to such Gram-positive bacterial (e.g., S. pneumoniae) infection comprising administering to said animal a therapeutic or prophylactic amount of the compositions of the invention. For example, the invention includes methods for the therapeutic or prophylactic treatment of a S. pneumoniae infection in an animal susceptible to streptococcal infection comprising administering to said animal a therapeutic or prophylactic amount of the compositions of the invention.

The invention also provides compositions of the invention for use of the compositions described herein as a medicament. In some embodiments the medicament elicits an immune response in a mammal (i.e., it is an immunogenic composition). In some embodiments the medicament is a vaccine.

The invention also provides the use of the compositions of the invention in the manufacture of a medicament for eliciting an immune response in a mammal. In some embodiments the medicament is a vaccine.

The invention also provides kits comprising one or more containers of compositions of the invention. Compositions can be in liquid form or can be lyophilized, as can individual antigens. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle). The composition may comprise a first component comprising one or more Gram-positive (e.g., S. pneumoniae) pili or pilus proteins. In some embodiments, the Gram-positive pili or pilus proteins are in an oligomeric or hyperoligomeric form.

The kit can further comprise a second container comprising a pharmaceutically-acceptable buffer, such as phosphate-buffered saline, Ringer's solution, or dextrose solution. It can also contain other materials useful to the end-user, including other buffers, diluents, filters, needles, and syringes. The kit can also comprise a second or third container with another active agent, for example, an antibiotic.

The kit can also comprise a package insert containing written instructions for methods of inducing immunity against a Gram-positive bacterium (e.g., S. pneumoniae) or for treating Gram-positive bacterial infections. The package insert can be an unapproved draft package insert or can be a package insert approved by the Food and Drug Administration (FDA) or other regulatory body.

The invention also provides a delivery device pre-filled with the immunogenic compositions of the invention.

The invention also provides methods for inducing an immune response in a mammal comprising the step of administering an effective amount of a composition of the invention. The immune response is, in some embodiments, protective and, in some embodiments, involves antibodies and/or cell-mediated immunity. This immune response will preferably induce long lasting (e.g., neutralizing) antibodies and a cell mediated immunity that can quickly respond upon exposure to one or more Gram-positive (e.g., S. pneumoniae) antigens. The method may raise a booster response.

The invention provides a method of neutralizing a Gram-positive bacterial (e.g., S. pneumoniae) infection in a mammal comprising administering to the mammal an effective amount of the immunogenic compositions of the invention, a vaccine of the invention, or antibodies which recognize an immunogenic composition of the invention.

In some embodiments the mammal is a human. Where the vaccine is for prophylactic use, the human can be a male or a female (either of child bearing age or a teenager). Alternatively, the human may be elderly (e.g., over the age of 50, 55, 60, 65, 70 or 75) and may have an underlying disease such as diabetes or cancer. In some embodiments, the human is a pregnant female or an elderly adult.

In some embodiments these uses and methods are for the prevention and/or treatment of a disease caused by a Gram-positive bacterium (e.g., S. pneumoniae). The compositions may also be effective against other streptococcal bacteria. The compositions may also be effective against other Gram positive bacteria.

One method of checking efficacy of therapeutic treatment involves monitoring Gram-positive (e.g., S. pneumoniae) bacterial infection after administration of one or more compositions of the invention. Immune responses against the Gram-positive (e.g., S. pneumoniae) antigens in the compositions of the invention can be monitored after administration of the composition(s).

One non-limiting method of assessing the immunogenicity of the component proteins of the immunogenic compositions of the present invention is to express the proteins recombinantly and to screen patient sera or mucosal secretions by immunoblot. A positive reaction between the protein and the patient serum indicates that the patient has previously mounted an immune response to the protein in question—that is, the protein is an immunogen. This method may also be used to identify immunodominant proteins and/or epitopes.

Another non-limiting method of checking efficacy of therapeutic treatment involves monitoring Gram-positive bacterial (e.g., S pneumoniae) infection after administration of the compositions of the invention. One means of checking efficacy of prophylactic treatment involves monitoring immune responses both systemically (such as monitoring the level of IgG1 and IgG2a production) and mucosally (such as monitoring the level of IgA production) against the Gram-positive (e.g., S. pneumoniae) antigens in the compositions of the invention after administration of the composition. Typically, Gram-positive bacteria serum specific antibody responses are determined post-immunization but pre-challenge.

The vaccine compositions of the present invention can, in some embodiments, be evaluated in in vitro and in vivo animal models prior to host, e.g., human, administration.

The efficacy of immunogenic compositions of the invention can also be determined in vivo by challenging animal models of Gram-positive bacteria (e.g., S. pneumoniae) infection, e.g., guinea pigs or mice, with the immunogenic compositions. The immunogenic compositions may or may not be derived from the same serotypes as the challenge serotypes. In some embodiments the immunogenic compositions are derivable from the same serotypes as the challenge serotypes. In some embodiments, the immunogenic composition and/or the challenge serotypes are derivable from the group of Gram-positive (e.g., S. pneumoniae) serotypes. [000185] In vivo efficacy models include but are not limited to: (i) A murine infection model using human Gram-positive bacteria (e.g., S. pneumoniae) serotypes; (ii) a murine disease model which is a murine model using a mouse-adapted Gram-positive bacteria (e.g., S. pneumoniae) strain, such as those strains which are particularly virulent in mice and (iii) a primate model using human Gram-positive bacteria (e.g., S. pneumoniae) isolates.

The immune response may be one or both of a TH1 immune response and a TH2 response.

The immune response may be an improved or an enhanced or an altered immune response.

The immune response may be one or both of a systemic and a mucosal immune response.

In some embodiments the immune response is an enhanced systemic and/or mucosal response.

An enhanced systemic and/or mucosal immunity is reflected in an enhanced TH1 and/or TH2 immune response. In some embodiments, the enhanced immune response includes an increase in the production of IgG1 and/or IgG2a and/or IgA.

In some embodiments the mucosal immune response is a TH2 immune response. In some embodiments, the mucosal immune response includes an increase in the production of IgA.

Activated TH2 cells enhance antibody production and are therefore of value in responding to extracellular infections. Activated TH2 cells may secrete one or more of IL-4, IL-5, IL-6, and IL-10. A TH2 immune response may result in the production of IgG1, IgE, IgA and memory B cells for future protection.

A TH2 immune response may include one or more of an increase in one or more of the cytokines associated with a TH2 immune response (such as IL-4, IL-5, IL-6 and IL-10), or an increase in the production of IgG1, IgE, IgA and memory B cells. In some embodiments, the enhanced TH2 immune response will include an increase in IgG1 production.

A TH1 immune response may include one or more of an increase in CTLs, an increase in one or more of the cytokines associated with a TH1 immune response (such as IL-2, IFNγ, and TNFβ), an increase in activated macrophages, an increase in NK activity, or an increase in the production of IgG2. In some embodiments, the enhanced TH1 immune response will include an increase in IgG2 production.

Immunogenic compositions of the invention, in particular, immunogenic compositions comprising one or more Gram-positive (e.g., S. pneumoniae) pilus antigens of the present invention may be used either alone or in combination with other antigens optionally with an immunoregulatory agent capable of eliciting a Th1 and/or Th2 response.

Compositions of the invention will generally be administered directly to a patient. Certain routes may be favored for certain compositions, as resulting in the generation of a more effective immune response, preferably a CMI response, or as being less likely to induce side effects, or as being easier for administration. Direct delivery may be accomplished by parenteral injection (e.g. subcutaneously, intraperitoneally, intradermally, intravenously, intramuscularly, or to the interstitial space of a tissue), or by rectal, oral (e.g. tablet, spray), vaginal, topical, transdermal (e.g. see WO 99/27961) or transcutaneous (e.g., see WO 02/074244 and WO 02/064162), intranasal (e.g., see WO03/028760), ocular, aural, pulmonary or other mucosal administration.

In some embodiments the invention can be used to elicit systemic and/or mucosal immunity.

In some embodiments, the immunogenic composition comprises one or more Gram-positive (e.g., S. pneumoniae) pilus antigen(s) which elicits a neutralizing antibody response and one or more Gram-positive (e.g., S. pneumoniae) pilus antigen(s) which elicit a cell mediated immune response. In some embodiments, the neutralizing antibody response prevents or inhibits an initial Gram-positive bacterial infection while the cell-mediated immune response capable of eliciting an enhanced Th1 cellular response prevents further spreading of the infection. The immunogenic composition may include one or more Gram-positive pilus antigens and one or more non-pilus Gram-positive antigens, e.g., cytoplasmic antigens. In some embodiments, the immunogenic composition comprises one or more Gram-positive surface antigens or the like and one or other antigens, such as a cytoplasmic antigen capable of eliciting a Th1 cellular response.

Dosage treatment can be a single dose schedule or a multiple dose schedule. Multiple doses may be used in a primary immunization schedule and/or in a booster immunization schedule. In a multiple dose schedule the various doses may be given by the same or different routes e.g. a parenteral prime and mucosal boost, a mucosal prime and parenteral boost, etc.

The compositions of the invention may be prepared in various forms. For example, the compositions may be prepared as injectables, either as liquid solutions or suspensions. Solid forms suitable for solution in, or suspension in, liquid vehicles prior to injection can also be prepared (e.g. a lyophilized composition). The composition may be prepared for topical administration e.g. as an ointment, cream or powder. The composition may be prepared for oral administration e.g. as a tablet or capsule, as a spray, or as a syrup (optionally flavored). The composition may be prepared for pulmonary administration e.g. as an inhaler, using a fine powder or a spray. The composition may be prepared as a suppository or pessary. The composition may be prepared for nasal, aural or ocular administration e.g. as drops. The composition may be in kit form, designed such that a combined composition is reconstituted just prior to administration to a patient. Such kits may comprise one or more antigens in liquid form and one or more lyophilized antigens.

Immunogenic compositions used as vaccines comprise an immunologically effective amount of antigen(s), as well as any other components, such as antibiotics, as needed. By ‘immunologically effective amount’, it is meant that the administration of that amount to an individual, either in a single dose or as part of a series, is effective for treatment or prevention, or increases a measurable immune response or prevents or reduces a clinical symptom. This amount varies depending upon the health and physical condition of the individual to be treated, age, the taxonomic group of individual to be treated (e.g. non-human primate, primate, etc.), the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials.

### Further Components of the Composition

The compositions of the invention will typically, in addition to the components mentioned above, comprise one or more ‘pharmaceutically acceptable carriers’, which include any carrier that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers, and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, and the like, may be present. A thorough discussion of pharmaceutically acceptable excipients is available in Gennaro (2000) Remington: The Science and Practice of Pharmacy. 20th ed., ISBN: 0683306472.

### Adjuvants

Vaccines of the invention may be administered in conjunction with other immunoregulatory agents. In particular, compositions will usually include one or more adjuvants. Adjuvants for use with the invention include, but are not limited to, one or more of the following set forth below:

A. Mineral Containing Compositions

Mineral containing compositions suitable for use as adjuvants in the invention include mineral salts, such as aluminum salts and calcium salts. The invention includes mineral salts such as hydroxides (e.g. oxyhydroxides), phosphates (e.g. hydroxyphosphates, orthophosphates), sulfates, etc. (e.g. see chapters 8 & 9 of Vaccine Design . . . (1995) eds. Powell & Newman. ISBN: 030644867X. Plenum.), or mixtures of different mineral compounds (e.g. a mixture of a phosphate and a hydroxide adjuvant, optionally with an excess of the phosphate), with the compounds taking any suitable form (e.g. gel, crystalline, amorphous, etc.), and with adsorption to the salt(s) being preferred. The mineral containing compositions may also be formulated as a particle of metal salt (WO 00/23105).

Aluminum salts may be included in vaccines of the invention such that the dose of Al³⁺ is between 0.2 and 1.0 mg per dose.

B. Oil-Emulsions

Oil-emulsion compositions suitable for use as adjuvants in the invention include squalene-water emulsions, such as MF59 (5% Squalene, 0.5% Tween™ 80, and 0.5% Span™ 85, formulated into submicron particles using a microfluidizer). See WO90/14837. See also, Podda, “The adjuvanted influenza vaccines with novel adjuvants: experience with the MF59-adjuvanted vaccine”, Vaccine (2001) 19: 2673-2680; Frey et al., “Comparison of the safety, tolerability, and immunogenicity of a MF59-adjuvanted influenza vaccine and a non-adjuvanted influenza vaccine in non-elderly adults”, Vaccine (2003) 21:4234-4237. MF59 is used as the adjuvant in the FLUAD™ influenza virus trivalent subunit vaccine.

In some embodiments adjuvants for use in the compositions are submicron oil-in-water emulsions. In some embodiments submicron oil-in-water emulsions for use herein are squalene/water emulsions optionally containing varying amounts of MTP-PE, such as a submicron oil-in-water emulsion containing 4-5% w/v squalene, 0.25-1.0% w/v Tween™ 80 (polyoxyelthylenesorbitan monooleate), and/or 0.25-1.0% Span™ 85 (sorbitan trioleate), and, optionally, N-acetylmuramyl-L-alanyl-D-isogluatminyl-L-alanine-2-(1′-2′-dipalmitoyl-sn-glycero-3-huydroxyphosphophoryloxy)-ethylamine (MTP-PE), for example, the submicron oil-in-water emulsion known as “MF59” (International Publication No. WO 90/14837; U.S. Pat. Nos. 6,299,884 and 6,451,325, incorporated herein by reference in their entireties; and Ott et al., “MF59—Design and Evaluation of a Safe and Potent Adjuvant for Human Vaccines” in Vaccine Design: The Subunit and Adjuvant Approach (Powell, M. F. and Newman, M. J. eds.) Plenum Press, New York, 1995, pp. 277-296). MF59 contains 4-5% w/v Squalene (e.g. 4.3%), 0.25-0.5% w/v Tween 80™, and 0.5% w/v Span 85™ and optionally contains various amounts of MTP-PE, formulated into submicron particles using a microfluidizer such as Model 110Y microfluidizer (Microfluidics, Newton, Mass.). For example, MTP-PE may be present in an amount of about 0-500 μg/dose, about 0-250 μg/dose and about 0-100 μg/dose. As used herein, the term “MF59-0” refers to the above submicron oil-in-water emulsion lacking MTP-PE, while the term MF59-MTP denotes a formulation that contains MTP-PE. For instance, “MF59-100” contains 100 μg MTP-PE per dose, and so on. MF69, another submicron oil-in-water emulsion for use herein, contains 4.3% w/v squalene, 0.25% w/v Tween 80™, and 0.75% w/v Span 85™ and optionally MTP-PE. Yet another submicron oil-in-water emulsion is MF75, also known as SAF, containing 10% squalene, 0.4% Tween 80™, 5% pluronic-blocked polymer L121, and thr-MDP, also microfluidized into a submicron emulsion. MF75-MTP denotes an MF75 formulation that includes MTP, such as from 100-400 gg MTP-PE per dose.

Submicron oil-in-water emulsions, methods of making the same and immunostimulating agents, such as muramyl peptides, for use in the compositions, are described in detail in International Publication No. WO 90/14837 and U.S. Pat. Nos. 6,299,884 and 6,451,325, incorporated herein by reference in their entireties.

Complete Freund's adjuvant (CFA) and incomplete Freund's adjuvant (IFA) may also be used as adjuvants in the invention.

C. Saponin Formulations

Saponin formulations may also be used as adjuvants in the invention. Saponins are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs.

Saponin compositions have been purified using High Performance Thin Layer Chromatography (HP-LC) and Reversed Phase High Performance Liquid Chromatography (RP-HPLC). Specific purified fractions using these techniques have been identified, including QS7, QS17, QS18, QS21, QH-A, QH-B and QH-C. Preferably, the saponin is QS21. A method of production of QS21 is disclosed in U.S. Pat. No. 5,057,540. Saponin formulations may also comprise a sterol, such as cholesterol (see WO96/33739).

Combinations of saponins and cholesterols can be used to form unique particles called Immunostimulating Complexs (ISCOMs). ISCOMs typically also include a phospholipid such as phosphatidylethanolamine or phosphatidylcholine. Any known saponin can be used in ISCOMs. In some embodiments, the ISCOM includes one or more of Quil A, QHA and QHC. ISCOMs are further described in EP0109942, WO 96/11711 and WO 96/33739. Optionally, the ISCOMS may be devoid of additional detergent. See WO 00/07621.

A review of the development of saponin based adjuvants can be found at Barr, et al., “ISCOMs and other saponin based adjuvants”, Advanced Drug Delivery Reviews (1998) 32:247-271. See also Sjolander, et al., “Uptake and adjuvant activity of orally delivered saponin and ISCOM vaccines”, Advanced Drug Delivery Reviews (1998) 32:321-338.

D. Virosomes and Virus Like Particles (VLPs)

Virosomes and Virus Like Particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in WO 03/024480, WO 03/024481, and Niikura et al., “Chimeric Recombinant Hepatitis E Virus-Like Particles as an Oral Vaccine Vehicle Presenting Foreign Epitopes”, Virology (2002) 293:273-280; Lenz et al., “Papillomarivurs-Like Particles Induce Acute Activation of Dendritic Cells”, Journal of Immunology (2001) 5246-5355; Pinto, et al., “Cellular Immune Responses to Human Papillomavirus (HPV)-16 L1 Healthy Volunteers Immunized with Recombinant HPV-16 L1 Virus-Like Particles”, Journal of Infectious Diseases (2003) 188:327-338; and Gerber et al., “Human Papillomavirus Virus-Like Particles Are Efficient Oral Immunogens when Coadministered with Escherichia coli Heat-Labile Enterotoxin Mutant R192G or CpG”, Journal of Virology (2001) 75(10):4752-4760. Virosomes are discussed further in, for example, Gluck et al., “New Technology Platforms in the Development of Vaccines for the Future”, Vaccine (2002) 20:B10-B16. Immunopotentiating reconstituted influenza virosomes (IRIV) are used as the subunit antigen delivery system in the intranasal trivalent INFLEXAL™ product (Mischler & Metcalfe (2002) Vaccine 20 Suppl 5:B17-23) and the INFLUVAC PLUS™ product.

E. Bacterial or Microbial Derivatives

In some embodiments adjuvants suitable for use in the invention include bacterial or microbial derivatives such as:

(1) Non-Toxic Derivatives of Enterobacterial Lipopolysaccharide (LPS)

Such derivatives include Monophosphoryl lipid A (MPL) and 3-O-deacylated MPL (3dMPL). 3dMPL is a mixture of 3 De-O-acylated monophosphoryl lipid A with 4, 5 or 6 acylated chains. A non-limiting example of a “small particle” form of 3 De-O-acylated monophosphoryl lipid A is disclosed in EP 0 689 454. Such “small particles” of 3dMPL are small enough to be sterile filtered through a 0.22 micron membrane (see EP 0 689 454). Other non-toxic LPS derivatives include monophosphoryl lipid A mimics, such as aminoalkyl glucosaminide phosphate derivatives e.g. RC-529. See Johnson et al. (1999) Bioorg Med Chem Lett 9:2273-2278.

(2) Lipid A Derivatives

Lipid A derivatives include derivatives of lipid A from Escherichia coli such as OM-174. OM-174 is described for example in Meraldi et al., “OM-174, a New Adjuvant with a Potential for Human Use, Induces a Protective Response with Administered with the Synthetic C-Terminal Fragment 242-310 from the circumsporozoite protein of Plasmodium berghei”, Vaccine (2003) 21:2485-2491; and Pajak, et al., “The Adjuvant OM-174 induces both the migration and maturation of murine dendritic cells in vivo”, Vaccine (2003) 21:836-842.

(3) Immunostimulatory Oligonucleotides

Immunostimulatory oligonucleotides suitable for use as adjuvants in the invention include nucleotide sequences containing a CpG motif (a sequence containing an unmethylated cytosine followed by guanosine and linked by a phosphate bond). Bacterial double stranded RNA or oligonucleotides containing palindromic or poly(dG) sequences have also been shown to be immunostimulatory.

The CpG's can include nucleotide modifications/analogs such as phosphorothioate modifications and can be double-stranded or single-stranded. Optionally, the guanosine may be replaced with an analog such as 2′-deoxy-7-deazaguanosine. See Kandimalla, et al., “Divergent synthetic nucleotide motif recognition pattern: design and development of potent immunomodulatory oligodeoxyribonucleotide agents with distinct cytokine induction profiles”, Nucleic Acids Research (2003) 31(9): 2393-2400; WO02/26757 and WO99/62923 for examples of possible analog substitutions. The adjuvant effect of CpG oligonucleotides is further discussed in Krieg, “CpG motifs: the active ingredient in bacterial extracts?”, Nature Medicine (2003) 9(7): 831-835; McCluskie, et al., “Parenteral and mucosal prime-boost immunization strategies in mice with hepatitis B surface antigen and CpG DNA”, FEMS Immunology and Medical Microbiology (2002) 32:179-185; WO98/40100; U.S. Pat. No. 6,207,646; U.S. Pat. No. 6,239,116 and U.S. Pat. No. 6,429,199.

The CpG sequence may be directed to TLR9, such as the motif GTCGTT (SEQ ID NO:54) or TTCGTT (SEQ ID NO:55). See Kandimalla, et al., “Toll-like receptor 9: modulation of recognition and cytokine induction by novel synthetic CpG DNAs”, Biochemical Society Transactions (2003) 31 (part 3): 654-658. The CpG sequence may be specific for inducing a Th1 immune response, such as a CpG-A ODN, or it may be more specific for inducing a B cell response, such a CpG-B ODN. CpG-A and CpG-B ODNs are discussed in Blackwell, et al., “CpG-A-Induced Monocyte IFN-gamma-Inducible Protein-10 Production is Regulated by Plasmacytoid Dendritic Cell Derived IFN-alpha”, J. Immunol. (2003) 170(8):4061-4068; Krieg, “From A to Z on CpG”, TRENDS in Immunology (2002) 23(2): 64-65 and WO01/95935. Preferably, the CpG is a CpG-A ODN.

In some embodiments, the CpG oligonucleotide is constructed so that the 5′ end is accessible for receptor recognition. Optionally, two CpG oligonucleotide sequences may be attached at their 3′ ends to form “immunomers”. See, for example, Kandimalla, et al., “Secondary structures in CpG oligonucleotides affect immunostimulatory activity”, BBRC (2003) 306:948-953; Kandimalla, et al., “Toll-like receptor 9: modulation of recognition and cytokine induction by novel synthetic GpG DNAs”, Biochemical Society Transactions (2003) 31(part 3):664-658; Bhagat et al., “CpG penta- and hexadeoxyribonucleotides as potent immunomodulatory agents” BBRC (2003) 300:853-861 and WO 03/035836.

(4) ADP-Ribosylating Toxins and Detoxified Derivatives Thereof.

Bacterial ADP-ribosylating toxins and detoxified derivatives thereof may be used as adjuvants in the invention. In some embodiments, the protein is derived from E. coli (i.e., E. coli heat labile enterotoxin “LT”), cholera (“CT”), or pertussis (“PT”). The use of detoxified ADP-ribosylating toxins as mucosal adjuvants is described in WO95/17211 and as parenteral adjuvants in WO98/42375. In some embodiments, the adjuvant is a detoxified LT mutant such as LT-K63, LT-R72, and LTR192G. The use of ADP-ribosylating toxins and detoxified derivatives thereof, particularly LT-K63 and LT-R72, as adjuvants can be found in the following references, each of which is specifically incorporated by reference herein in their entirety: Beignon, et al., “The LTR72 Mutant of Heat-Labile Enterotoxin of Escherichia coli Enhances the Ability of Peptide Antigens to Elicit CD4+ T Cells and Secrete Gamma Interferon after Coapplication onto Bare Skin”, Infection and Immunity (2002) 70(6):3012-3019; Pizza, et al., “Mucosal vaccines: non toxic derivatives of LT and CT as mucosal adjuvants”, Vaccine (2001) 19:2534-2541; Pizza, et al., “LTK63 and LTR72, two mucosal adjuvants ready for clinical trials” Int. J. Med. Microbiol (2000) 290(4-5):455-461; Scharton-Kersten et al., “Transcutaneous Immunization with Bacterial ADP-Ribosylating Exotoxins, Subunits and Unrelated Adjuvants”, Infection and Immunity (2000) 68(9):5306-5313; Ryan et al., “Mutants of Escherichia coli Heat-Labile Toxin Act as Effective Mucosal Adjuvants for Nasal Delivery of an Acellular Pertussis Vaccine: Differential Effects of the Nontoxic AB Complex and Enzyme Activity on Th1 and Th2 Cells” Infection and Immunity (1999) 67(12):6270-6280; Partidos et al., “Heat-labile enterotoxin of Escherichia coli and its site-directed mutant LTK63 enhance the proliferative and cytotoxic T-cell responses to intranasally co-immunized synthetic peptides”, Immunol. Lett. (1999) 67(3):209-216; Peppoloni et al., “Mutants of the Escherichia coli heat-labile enterotoxin as safe and strong adjuvants for intranasal delivery of vaccines”, Vaccines (2003) 2(2):285-293; and Pine et al., (2002) “Intranasal immunization with influenza vaccine and a detoxified mutant of heat labile enterotoxin from Escherichia coli (LTK63)” J. Control Release (2002) 85(1-3):263-270. Numerical reference for amino acid substitutions is preferably based on the alignments of the A and B subunits of ADP-ribosylating toxins set forth in Domenighini et al., Mol. Microbiol (1995) 15(6):1165-1167, specifically incorporated herein by reference in its entirety.

F. Bioadhesives and Mucoadhesives

Bioadhesives and mucoadhesives may also be used as adjuvants in the invention. Suitable bioadhesives include esterified hyaluronic acid microspheres (Singh et al. (2001) J. Cont. Rele. 70:267-276) or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention. E.g., see WO 99/27960.

G. Microparticles

Microparticles may also be used as adjuvants in the invention. Microparticles (i.e. particles of ˜100 nm to ˜150 μin diameter, of ˜200 nm to ˜30 nm in diameter, and of ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

H. Liposomes

Examples of liposome formulations suitable for use as adjuvants are described in U.S. Pat. No. 6,090,406, U.S. Pat. No. 5,916,588, and EP 0 626 169.

I. Polyoxyethylene Ether and Polyoxyethylene Ester Formulations

In some embodiments adjuvants suitable for use in the invention include polyoxyethylene ethers and polyoxyethylene esters. WO99/52549. Such formulations further include polyoxyethylene sorbitan ester surfactants in combination with an octoxynol (WO01/21207) as well as polyoxyethylene alkyl ethers or ester surfactants in combination with at least one additional non-ionic surfactant such as an octoxynol (WO 01/21152).

In some embodiments polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

J. Polyphosphazene (PCPP)

PCPP formulations are described, for example, in Andrianov et al., “Preparation of hydrogel microspheres by coacervation of aqueous polyphophazene solutions”, Biomaterials (1998) 19(1-3):109-115 and Payne et al., “Protein Release from Polyphosphazene Matrices”, Adv. Drug. Delivery Review (1998) 31(3):185-196.

K. Muramyl Peptides

Examples of muramyl peptides suitable for use as adjuvants in the invention include N-acetyl-muramyl-L-threonyl-D-isoglutamine (thr-MDP), N-acetyl-normuramyl-1-alanyl-d-isoglutamine (nor-MDP), and N-acetylmuramyl-1-alanyl-d-isoglutaminyl-1-alanine-2-(1′-2′-dipalmitoyl-sn-glycero-3-hydroxyphosphoryloxy)-ethylamine MTP-PE).

L. Imidazoquinolone Compounds.

Examples of imidazoquinolone compounds suitable for use as adjuvants in the invention include, without limitation, Imiquamod and its homologues, described further in Stanley, “Imiquimod and the imidazoquinolones: mechanism of action and therapeutic potential” Clin Exp Dermatol (2002) 27(7):571-577 and Jones, “Resiquimod 3M”, Curr Opin Investig Drugs (2003) 4(2):214-218.

The invention also provides compositions comprising combinations of the adjuvants identified above. For example, the following adjuvant compositions are non-limiting examples of adjuvant combinations which may be used in the invention:

(1) a saponin and an oil-in-water emulsion (WO 99/11241);

(2) a saponin (e.g., QS21)+a non-toxic LPS derivative (e.g. 3dMPL) (see WO 94/00153);

(3) a saponin (e.g., QS21)+a non-toxic LPS derivative (e.g. 3dMPL)+a cholesterol;

(4) a saponin (e.g. QS21)+3dMPL+IL-12 (optionally+a sterol) (WO 98/57659);

(5) combinations of 3dMPL with, for example, QS21 and/or oil-in-water emulsions (See European patent applications 0835318, 0735898 and 0761231);

(6) SAF, containing 10% Squalane, 0.4% Tween 80, 5% pluronic-block polymer L121, and thr-MDP, either microfluidized into a submicron emulsion or vortexed to generate a larger particle size emulsion.

(7) Ribi™ adjuvant system (RAS), (Ribi Immunochem) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™);

(8) one or more mineral salts (such as an aluminum salt)+a non-toxic derivative of LPS (such as 3dPML).

(9) one or more mineral salts (such as an aluminum salt)+an immunostimulatory oligonucleotide (such as a nucleotide sequence including a CpG motif). Combination No. (9) is a preferred adjuvant combination.

M. Human Immunomodulators

Human immunomodulators suitable for use as adjuvants in the invention include cytokines, such as interleukins (e.g. IL-1, IL-2, IL-4, IL-5, IL-6, IL-7, IL-12, etc.), interferons (e.g. interferon-γ), macrophage colony stimulating factor, and tumor necrosis factor. [000233] In some embodiments aluminum salts and MF59 are preferred adjuvants for use with injectable influenza vaccines. In some embodiments bacterial toxins and bioadhesives are preferred adjuvants for use with mucosally-delivered vaccines, such as nasal vaccines.

The immunogenic compositions of the present invention may be administered in combination with an antibiotic treatment regime. In some embodiments, the antibiotic is administered prior to administration of the antigen of the invention or the composition comprising the one or more of the antigens of the invention.

In some embodiments, the antibiotic is administered subsequent to the administration of the one or more antigens of the invention or the composition comprising the one or more antigens of the invention. Examples of antibiotics suitable for use in the treatment streptococcal infections include but are not limited to penicillin or a derivative thereof or clindamycin or the like.

The invention is further illustrated, without limitation, by the following examples.

### EXAMPLES

### Example 1

### Materials and Methods

Construction of Pneumococcal Mutants

Pneumococcal strains and deletion mutants created in these backgrounds are described in Table 1. PCR ligation mutagenesis (23) was used to create knockout mutants of T4 and ST162^{19F}. Fragments upstream and downstream of the target genes were amplified with specific primer pairs. The upstream fragments were constructed with ApaI sites and the downstream fragments with BamHI sites. Primers used for construction and screening of deletion alleles are listed in Table 2. The PCR products (1,000 bp) were digested with corresponding restriction enzymes, purified, and ligated with the erm cassette (1,306 bp) (GenBank accession no. AB057644) or the Kan-rpsL cassette, Janus (24) (1,368 bp) containing ApaI and BamHI sites. The ligation mix was then transformed as described in (25) into the recipient pneumococcal strain and plated on blood agar plates containing either erythromycin (1 μg/ml) or kanamycin (400 μg/ml). The correct insertion was confirmed by PCR and sequencing.

TABLE 1
S. pneumoniae strains used

| Strain | Relevant characteristics | Source |
|---|---|---|
| T4 | Type 4 strain TIGR4 | tigr.org |
| T4Δ(rlrA) | rlrA::erm (Em^{R}) | herein |
| T4Δ(rrgA-srtD) | rrgABC-srtBCD::erm (Em^{R}) | herein |
| T4Δ(mgrA) | mgrA::erm (Em^{R}) | herein |
| T4Δ(rrgA-srtD, mgrA) | (rrgABC-srtBCD::erm)::(mgrA::km-rpsL) (Em^{R}, | herein |
| | Km^{R}) | |
| T4Δ(rrgA-srtD)∇(rrgA- | T4Δ(rrgA-srtD) where (rrgABC-srtBCD)::erm (Em^{R}) | herein and |
| srtD) | was replaced by (rrgABC-srtBCD-Km) (Km^{R}) | (24) |
| T4R | Cm^{R} inactivation of cps4A in T4 | (27, 28) |
| T4RΔ(rrgA-srtD) | rrgABC-srtBCD::erm (Em^{R}) in T4R | herein |
| ST162^{19F} | Clinical isolate of type 19F, excellent colonizer in | (5) |
| | mice | |
| ST162^{19F} Δ(rrgA-srtD) | rrgABC-srtBCD::erm (Em^{R}) | herein |
| ST162^{19F} Δ(mgrA) | mgrA::erm (Em^{R}) | herein |
| ST162^{19F} Δ(rrgA-srtD, | rrgABC-srtBCDr.erm (Em^{R}), mgrA::km-rpsL (Km^{R}) | herein |
| mgrA) | | |
| D39 | Type 2 strain lacking the rlrA islet | (29) |
| D39∇(rrgA-srtD) | rlrA islet IS161::magellan5 (Spc^{R}, Sm^{R}) | herein |
| D39∇(rrgA-srtD)Δ(rlrA) | rlrA islet IS167::magellan5 rlrA::magellan2 (Spc^{R}, | herein |
| | Cm^{R}, Sm^{R}) | |

Em^{R}, erythromycin-resistant; Km^{R}, Kanamycin-resistant; Spc^{R}, spectinomycin-resistant; Sm^{R}, streptomycin-resistant; Cm^{R}, chloramphenicol-resistant.

TABLE 2
Primers and restriction enzymes used for
creation of mutants

| | | Restric- | |
|---|---|---|---|
| | | tion | Sequence |
| Gene | Name | enzyme | (5′ to 3′) |
| erm | ErmF | ApaI | TTTTTGGGCCCTTCGTGTTCG |
| cassette | | | TGCTGACTTGC |
| | | | (SEQ ID NO: 18) |
| | ErmR | BamHI | TTTTTGGATCCGATGTTGCTG |
| | | | ATTAAGACGAGC |
| | | | (SEQ ID NO: 19) |
| | ErmstartR | | AACTTCTTTTACGTTTCCGCC |
| | | | (SEQ ID NO: 20) |
| | ErmslutF | | ACCGAAAGACAGACGAGCC |
| | | | (SEQ ID NO: 21) |
| Kan-rpsL | Kan-Bam | BamHI | TTGGATCCCTTTAAATACTGT |
| cassette | | | AGAAAAGAGGA |
| | | | (SEQ ID NO: 22) |
| | Kan-Apa | ApaI | TTGGGCCCTAAAACAATTCAT |
| | | | CCAGTAAAAT |
| | | | (SEQ ID NO: 23) |
| | Dam406 | BamHI | TCTATGCCTATTCCAGAGGAA |
| | | | ATGGATCGGATC |
| | | | (SEQ ID NO: 24) |
| | Dam351 | ApaI | CTAGGGCCCTTTCCTTATGCT |
| | | | TTTGGAC |
| | | | (SEQ ID NO: 25) |
| | Dam407 | | AGGAGACATTCCTTCCGTATC |
| | | | T |
| | | | (SEQ ID NO: 26) |
| | Dam352 | | CAAGAGCACAGCGTGGTGCT |
| | | | (SEQ ID NO: 27) |
| T4Δ(rrgA- | RrgA P1 | | CAAGGTCCAAACCTACTGAAC |
| srtD) | | | (SEQ ID NO: 28) |
| | RrgA P2 | ApaI | GCGGGCCCCTGAGATATACAG |
| | | | CACAGTCC |
| | | | (SEQ ID NO: 29) |
| | SrtD P3 | BamHI | CGGGATCCCTGGCATTTCTGG |
| | | | GAATCCTG |
| | | | (SEQ ID NO: 30) |
| | SrtD P4 | | CGTTTCAAGTGCTATCACTGT |
| | | | TC |
| | | | (SEQ ID NO: 31) |
| T4Δ(mgrA) | MgrA P1 | | ATATAACATGAACAGTTGGGT |
| | | | TCTTG |
| | | | (SEQ ID NO: 32) |
| | MgrA P2 | ApaI | ATATAGGGCCCAACCTCTTGC |
| | | | AATTATACCACA |
| | | | (SEQ ID NO: 33) |
| | MgrA P3 | BamHI | ATATAGGATCCCGCGTTTGAA |
| | | | CTGTACCTCAA |
| | | | (SEQ ID NO: 34) |
| | MgrA P4 | | ATATACAGTAACTGTCTCATC |
| | | | CAAATC |
| | | | (SEQ ID NO: 35) |
| | MgrA C1 | | ATATACTGCTTCAATCCATTA |
| | | | GTTATTTC |
| | | | (SEQ ID NO: 36) |
| | MgrA C2 | | ATATATTGATTGTAAAAATTC |
| | | | CATCTATAG |
| | | | (SEQ ID NO: 37) |
| T4Δ(rrgA- | Rev1 | BamHI | TTGGATCCTTATTTCCCTCGT |
| srtD) | | | AGTAAACGT |
| ∇(rrgA- | | | (SEQ ID NO: 38) |
| srtD) | | | |
| | Rev2 | ApaI | TTGGGCCCAAAGAAATGAAAG |
| | | | GAAAGCTAAGG |
| | | | (SEQ ID NO: 39) |
| ST162^{19F}Δ | RrgA P1 | | CAAGGTCCAAACCTACTGAAC |
| (rrgA-srtD) | | | (SEQ ID NO: 40) |
| | RgA P2 | ApaI | GCGGGCCCCTGAGATATACAG |
| | | | CACAGTCC |
| | | | (SEQ ID NO: 41) |
| | SrtD P3 | BamHI | CGGGATCCCTGGCATTTCTGG |
| | | | GAATCCTG |
| | | | (SEQ ID NO: 42) |
| | SrtD P4 | | CGTTTCAAGTGCTATCACTGT |
| | | | TC |
| | | | (SEQ ID NO: 43) |
| | SrtD C2 | | GCCCCATCTTGCCCTCACTGC |
| | | | G |
| | | | (SEQ ID NO: 44) |
| ST162^{19F}Δ | MgrA P1 | | ATATAACATGAACAGTTGGGT |
| (mgrA) | | | TCTTG |
| | | | (SEQ ID NO: 45) |
| | MgrA P2 | ApaI | ATATAGGGCCCAACCTCTTGC |
| | | | AATTATACCACA |
| | | | (SEQ ID NO: 46) |
| | MgrA P3 | BamHI | ATATAGGATCCCGCGTTTGAA |
| | | | CTGTACCTCAA |
| | | | (SEQ ID NO: 47) |
| | MgrA P4 | | ATATACAGTAACTGTCTCATC |
| | | | CAAATC |
| | | | (SEQ ID NO: 48) |
| | MgrA C1 | | ATATACTGCTTCAATCCATTA |
| | | | GTTATTTC |
| | | | (SEQ ID NO: 49) |
| | MgrA C2 | | ATATATTGATTGTAAAAATTC |
| | | | CATCTATAG |
| | | | (SEQ ID NO: 50) |
| D39∇ | RLRAFR | | CGCGGATCCAAAGGAGAATCA |
| (rrgA-srt) | | | TCATGCTAAACAAATACATTG |
| Δ(rlrA) | | | A |
| | | | (SEQ ID NO: 51) |
| | RLRARX | | CCCTCTAGATTATAACAAATA |
| | | | GTGAGCCTT |
| | | | (SEQ ID NO: 52) |

To create an insertion mutant of D39 (serotype 2 strain) containing the rlrA islet, competent D39 cells were transformed with genomic DNA from CH155, a serotype 4 S. pneumoniae strain with a magellan5 transposon insertion in one of the IS1167 elements flanking the rlrA islet. The double recombination event was selected for by plating on spectinomycin, and islet presence was confirmed by PCR. To generate an rlrA mutant derivative of D39∇(rrgA-srtD), PCR amplification of the mutated region in the mutant serotype 4 strain was performed with primer pairs RLRAFR/RLRARX and the purified amplicon transformed into required serotype 2 background. The recombination event was selected for by plating on chloramphenicol for rlrA and confirmed by PCR.

Cloning, Expression, and Purification of RrgA, RrgB, and RrgC

Standard recombinant DNA techniques were used to construct all expression plasmids. pET 21b+ was purchased from Invitrogen. PCR was performed with Pfu Turbo Taq™ (Roche) during 25 cycles of amplification with genomic DNA. PCR products were purified, digested, ligated into a vector, transformed into E. coli TOPO10, and subsequently subcloned into E. coli BLR(DE3). Recombinant proteins were expressed and ₚurified from transformed bacteria according to the instructions of the manufacturer.

Animal Sera

Purified recombinant RrgA, RrgB, and RrgC were concentrated with a Centricon YM-30 spin column (Millipore) and subsequently used to immunize BALB/c mice (20 μg) and New Zealand White rabbits (100 μg).

Negative Staining

For negative staining, bacteria were grown on blood agar for up to 16 hours, and colonies were resuspended in 0.15 M sodium cacodylate buffer. An aliquot of 4 μl was added to a grid coated with a Formvar™ supporting film for 5 minutes. The excess solution was soaked off by a filter paper and the grid was stained with 0.5% uranyl acetate in water for 5 seconds and air-dried. The samples were examined in a Tecnai™ 10 electron microscope (Phillips) at 80 kV.

Immunoelectron Microscopy

S. pneumoniae was grown overnight in liquid THY medium. One milliliter of bacterial suspension with an OD₆₀₀ of 0.5 was centrifuged at 3,000 rpm at 4° C. and resuspended in 500 μl of sterile filtered PBS. Twenty microliters of sample was added to Formvar™-coated nickel grids and let stand for 5 minutes. The grids were subsequently fixed in 1% paraformaldehyde/PBS and incubated with 1:10 polyclonal mouse antibodies to RrgA, RrgB, or RrgC in blocking buffer (1% normal rabbit serum, 1% BSA, 1× PBS). Samples were washed five times for 5 minutes in blocking buffer and incubated with secondary gold-conjugated antibodies at 1:20 (goat anti-mouse IgG, 5-nm gold particles; goat anti-rabbit IgG; 10 nm). Samples were washed five times in blocking buffer for 5 minutes, and subsequently fixed for 30 minutes in 1% paraformaldehyde/PBS. Samples were washed in distilled water five times for 5 minutes and let dry. Grids were stained for 15 seconds with aqueous uranyl acetate and processed in a Tecnai™ high-field transmission electron microscope.

Western Blotting

Bacteria were grown on blood agar plates for up to 16 hours. Bacteria (30 mg wet weight) were resuspended in 1 ml of 50 mM Tris-HCl, pH 6.8, containing 400 units of Mutanolysin (Sigma) and incubated 2 hours at 37° C. After three cycles of freezing and thawing, cellular debris was removed by centrifugation at 13,000 rpm for 15 minutes. Fifty microliters of the supernatant was treated with NuPage™ sample buffer and mercaptoethanol for 10 minutes at 70° C., and 10 μl was loaded on a 4-12% or 3-8% NuPage Novex™ Bis-Tris Gel (Invitrogen). The electroblotting and detection with RrgB antibody (mouse immune sera) diluted 1:500 was performed according to the supplier's instructions.

A549 Adherence Assays

S. pneumoniae cells grown to mid-exponential phase (OD₆₀₀=0.3-0.4) were incubated with A549 cells for 30-40 minutes at 37° C. under a 5% CO₂/95% air atmosphere, and washed three times with PBS (pH 7.4) to remove nonadherent bacteria. For enumeration of adherent and/or internalized bacteria, epithelial cells were detached from the wells by treatment with 200 μl of 0.25% trypsin/1 mM EDTA and lysed by the addition of 800 μl of ice-cold 0.025% Triton™ X-100. Appropriate dilutions were plated on blood agar plates to count the number of bacteria adherent to the eukaryotic cells. The titer of adherent bacteria for each strain was compared to the input titer, and the percentage of adherent bacteria was determined.

For fluorescence microscopy, A549 monolayers were grown on coverslips in 24-well tissue culture plates. Infected cell layers on coverslips were fixed in 3% paraformaldehyde and labeled with antibodies after the 30- to 40-min incubation and washing with PBS. Bacteria were labeled with anti-capsular antibody and epithelial cells were visualized after permeabilization by staining F-actin with rhodamine-conjugated phalloidin. All experiments were performed in quadruplicate, and each experiment was replicated three times on different days.

Mouse Challenge

T4 and ST162^{19F} and their respective isogenic mutants were grown for 16 hours on blood agar plates at 37° C. under 5% CO₂. Colonies were taken directly from plates and resuspended gently in PBS to OD₆₂₀=0.5 or inoculated into semi-synthetic C+Y medium and grown to mid-logarithmic phase (OD₆₂₀=0.5) for intranasal inoculation, and OD₆₂₀=0.2 for intraperitoneal (i.p.) inoculation. Appropriate dilutions were made to obtain the desired concentration. Six- to 8-week-old C57BL/6 mice were used for intranasal and i.p. bacterial challenge of T4, and ST162^{19F} and their mutants as described in (5). D39 and its isogenic mutants were grown in THY broth supplemented with appropriate antibiotics. Six- to 10-week-old female CD1 (UK) mice (Charles River Laboratories) were used for intranasal challenge with 1×10⁷ bacteria.

For competition experiments, mutant and wild-type bacteria were mixed in a 1:1 ratio. The output of mutant cfu compared to the wild-type cfu was determined by selection on erythromycin, streptomycin, and/or chloramphenicol blood agar plates. In vivo competition indices (CI) were calculated as the ratio of mutant to wild-type output cfu divided by the mutant to wildtype input cfu.

Determination of TNF and IL-6 after i.p. challenge in serum was performed by using commercial ELISA kits (BD Biosciences).

Statistical Analysis

Data were analyzed for statistical significance by using GraphPad PRISM™ Version 4. Continuous variables were compared by using the t test or the nonparametric Mann-Whitney test. Statistical significance was defined as P<0.05.

FACS Analysis

S. pneumoniae [T4, ST1621^{9F}, T4Δ(mgrA), and T4Δ(rrgA-srtD)] isolates were grown in THY liquid culture overnight at 37° C. under 5% CO₂. Samples were diluted and allowed to grow to OD₆₂₀=0.250 (˜1×10⁸ per ml). Bacterial cultures were centrifuged at 3,000 rpm and resuspended in 1× PBS. Fifteen microliters of bacterial suspension was added to 96-well plates. Five microliters of 20% normal rabbit serum was added to each well, along with primary antibodies (anti-RrgB, PI anti-RrgB, Nm anti-961) at 1:3,200. Samples were incubated on ice for 30 minutes, after which 150 μl of blocking buffer (1% BSA/PBS) was added to the wells. The 96-well plate was centrifuged at 2,500 rpm for 5 minutes at 4° C. A secondary anti-mouse antibody labeled with phycoerythrin (Jackson ImmunoResearch) was added at a final concentration of 1:100, and the mixture was incubated for 30 minutes on ice. Then 150 μl of blocking buffer was added and samples were centrifuged as above. Samples were resuspended in 200 μl of 1% paraformaldehyde/PBS and analyzed on the FACSCaliber™ (Becton Dickinson).

Creation of Revertant in T4Δ(rrgA-srtD)

The rlrA islet was reintroduced into T4Δ(rrgA-srtD) by reintroducing the knocked-out genes together with a kanamycin cassette. The kanamycin cassette was first integrated downstream of the target genes in the wild-type T4 strain by PCR ligation mutagenesis. Chromosomal DNA from these mutants was used to transform the knockouts and restore the wild-type phenotype. In the first step, the kanamycin cassette was amplified from Janus (Sung et al., 2001, Appl. Environ. Microbiol., 67:5190-6) with the primers Kan-Apa and Kan-Bam, creating a PCR product with ApaI and BamHI termini. Fragments upstream and downstream of the target sites were amplified with primers pili-rev-1-4 for the T4Δ(rrgA-srtD) mutant. The upstream fragments were constructed with ApaI sites and the downstream fragments with BamHI sites. All fragments were digested with corresponding restriction endonucleases and ligations were performed with equimolar amounts of upstream fragment, downstream fragment, and kanamycin fragment. After transformation into T4, transformants were selected on blood agar plates containing 200 mg/L kanamycin. After control PCRs confirming the correct construction, chromosomal DNA from these transformants was used to transform T4Δ(rrgA-srtD). Again, selection was done on kanamycin-containing plates and the mutants were confirmed by checking for erythromycin sensitivity and pili expression.

### Example 2

### Evidence by Transmission Electron Microscopy for Pilus-Like Structures in Pneumococci

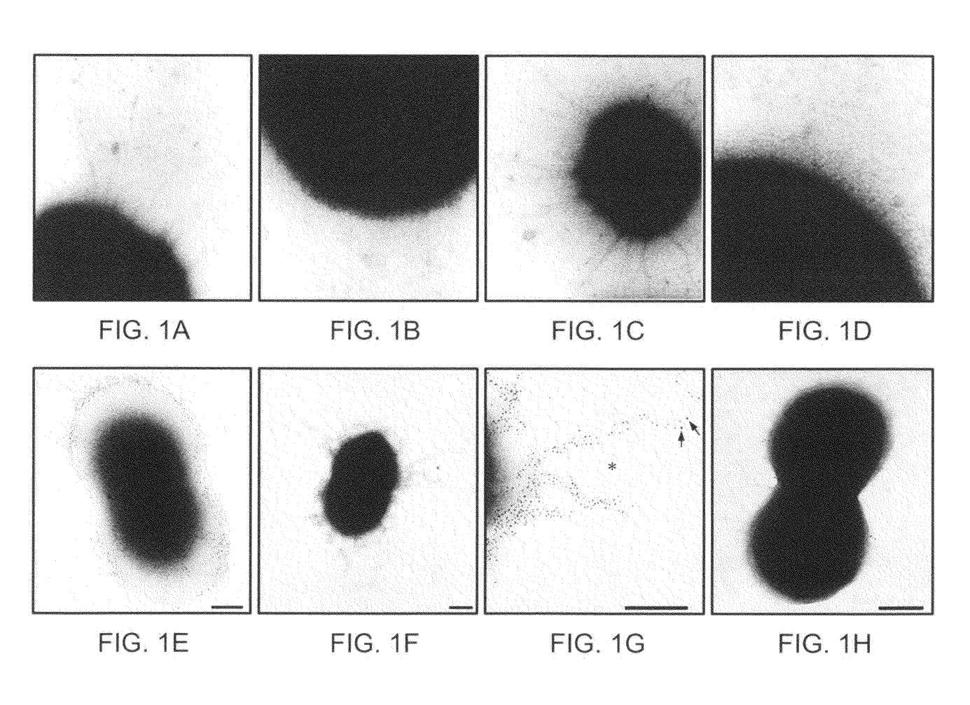

By transmission electron microscopy and negative staining, it was found that pneumococci cultivated for up to 16 hours on blood agar plates and in (C+Y) or (THY) medium express pilus-like structures. These structures were found on strain T4 (TIGR4), belonging to the highly invasive serotype 4 clone of multilocus sequence type ST205, as well as on a clinical isolate of type 19F, with multilocus sequence type 162 (strain ST162^{19F}) (FIG. 1A). This 19F clone is associated with both carriage and invasive disease in humans, and has been shown to be an efficient colonizer of the respiratory tract of C57BL/6 and BALB/c mice (5). Although a nonencapsulated mutant of T4 (T4R) was able to form pili, no pili were observed on the nonencapsulated laboratory strain R6.

### Example 3

### The rlrA Islet in the Pneumococcal Genome Encodes Pili-Like Structures

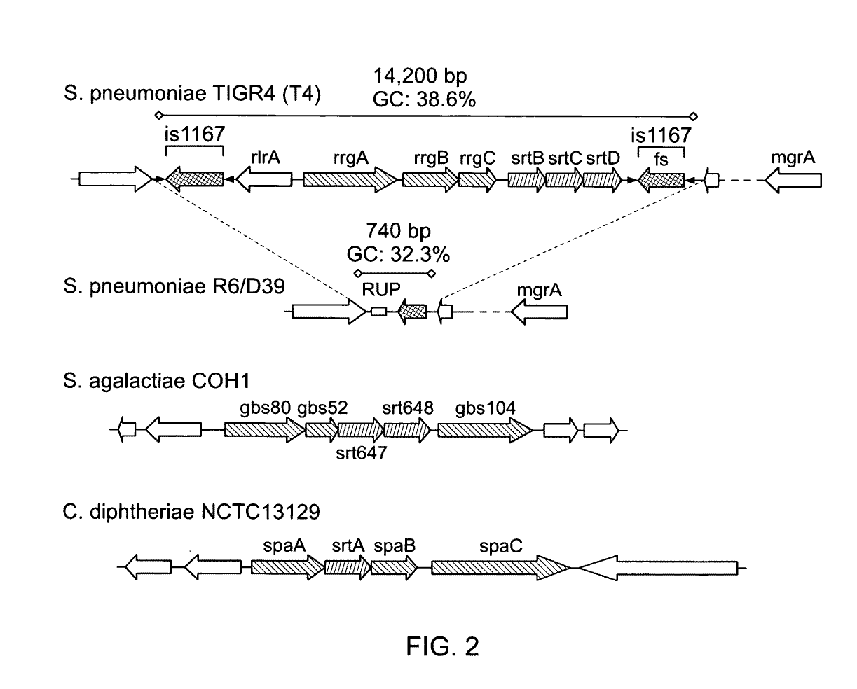

Comparison of the spaABC operon from Corynebacterium diphtheriae (12) and adhesion islet 1 from group B streptococci (16) revealed a cluster of putative pilus genes within the T4 genome (FIG. 2). The pilus genes are located in the previously described Streptococcus pneumoniae rlrA pathogenicity islet (18, 19). The pneumococcal rlrA islet consists of seven genes of which rrgA, rrgB, and rrgC are predicted to encode LPXTG-containing microbial surface components recognizing adhesive matrix molecules (MSCRAMMs) that bind to components of the extracellular matrix of the host (20). In addition, the rlrA islet also contains genes for three sortases, srtB, srtC, and srtD, as well as rlrA (rofA-like regulator), a positive regulator of the gene cluster (18) (FIG. 2). The genomic islet is flanked by IS1167 containing inverted repeats, characteristic of mobile genetic elements (FIG. 2). The sequenced strain R6 and its progenitor D39 are lacking the rlrA-pilus islet (FIG. 2). The transcriptional repressor mgrA is located external to the rlrA islet, and is involved in the regulation of the pilus genes (21). Sequence analysis after PCR amplification of the corresponding region in the clinical isolate ST162^{19F} of serotype 19F revealed a homologous gene cluster with 98% identity to the T4 rlrA islet. A small ORF of unknown function in T4 was however absent in the ST162^{19F} isolate. Knockout mutants deleted for the mgrA gene of T4 and ST162^{19F} were constructed by PCR ligation mutagenesis, thereby producing strains over-expressing the genes of the rlrA islet. In addition, we deleted the rrgA-srtD region in T4 (FIG. 1B) and ST162^{19F}, as well as in their respective mgrA derivatives. Upon negative staining and electron microscopy the T4 mgrA and ST162^{19F} mgrA mutants were found to produce abundant pili (FIG. 1C), whereas bacteria containing the rrgA-srtD deletion lacked pili altogether (FIG. 1D).

Antisera were raised against RrgA, RrgB, and RrgC proteins expressed in Escherichia coli, and used in immunogold labeling of the pilus expressed by T4. The RrgB antibodies decorated the entire pilus polymer (FIG. 1E-G). FACS analysis, making use of RrgB-specific antibodies, revealed that 84% and 90% of the cells of T4 and ST162^{19F}, respectively, expressed pili structures. In the mgrA mutant derivatives, almost all (99%) of the bacteria were piliated. Cells lacking the rlrA islet had no pili as measured by FACS analysis.

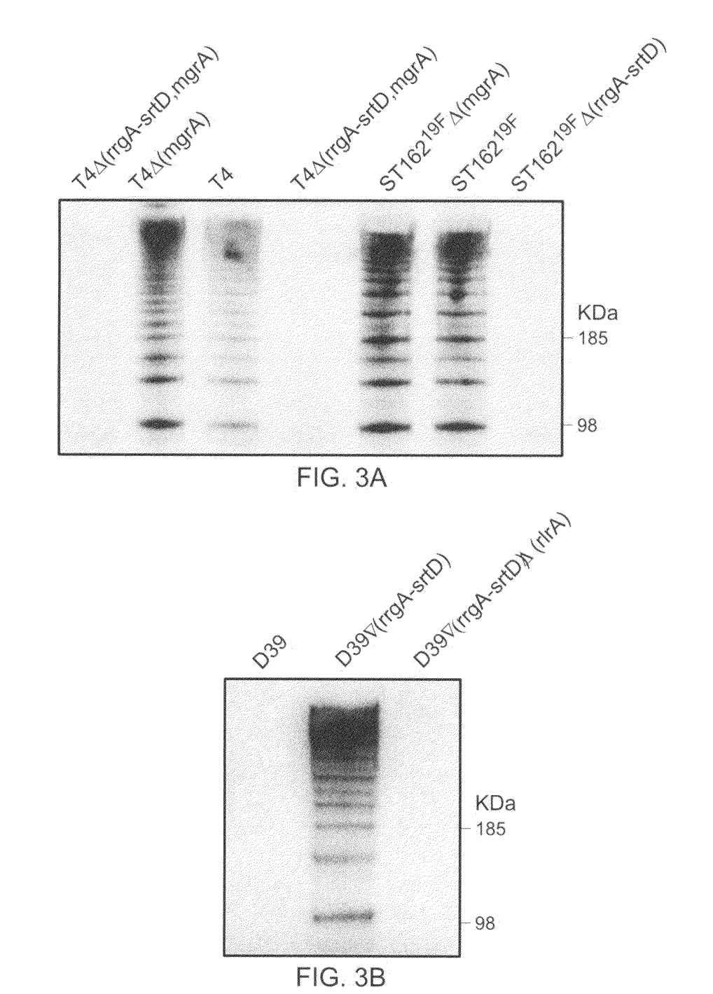

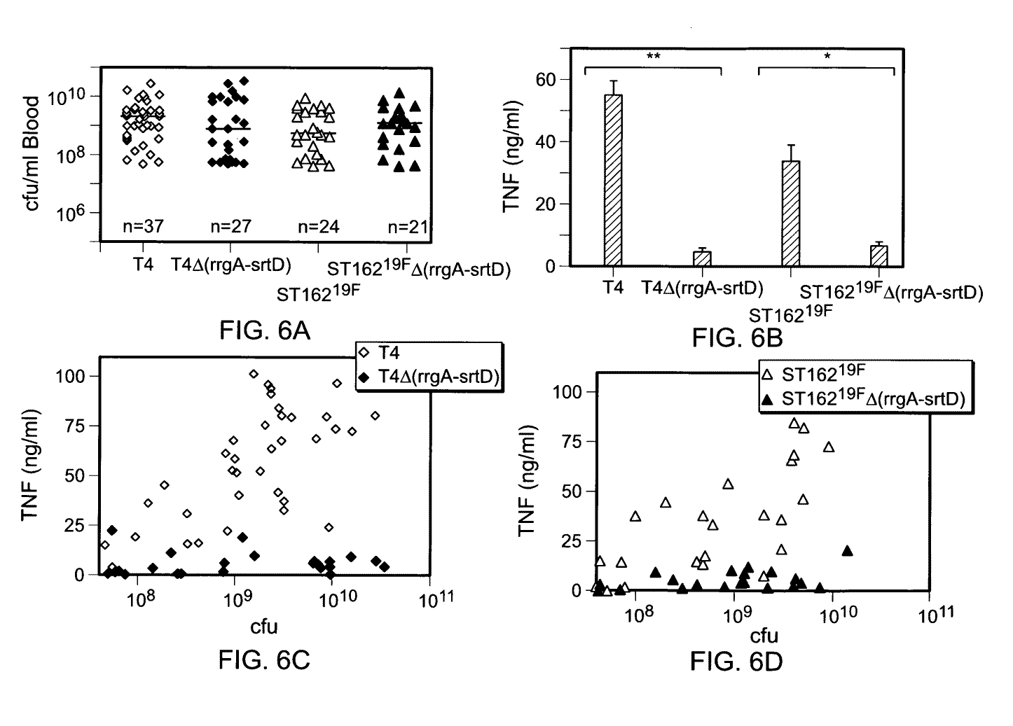

To verify the polymeric nature of the pili structures observed in T4 and ST162^{19F}, total extracts of these strains and their respective rrgA-srtD deletion derivatives were treated with mutanolysin, separated on 4-12% (FIG. 3A) and 3-8% (FIG. 3B) polyacrylamide gradient gels, and immunoblotted with antisera specific for RrgB. A ladder of high molecular weight (HMW) polymers ranging from <100 kDa to >1,000 kDa was observed, similar to those previously described in C. diphtheriae (12, 13). Even though equal amounts of protein extract were loaded onto the gel, the bands stained by the RrgB antibodies were more intense for the mgrA mutants than for their respective wild-type strains, supporting the data from transmission electron microscopy and FACS analysis that a greater percentage of pneumococci expressed pilus structures in the mgrA mutant background. As expected, the deletion mutants of rrgA-srtD, in T4 and ST162^{19F}, respectively, showed no RrgB-reactive bands (FIG. 3). However, when the pilus islet was reintroduced into the deletion mutant T4Δ(rrgA-srtD), Western blot analysis with the RrgB antiserum detected HMW polymers similar to those for the wild type T4 strain. By using Western blotting it was observed that pili were present in pneumococcal strains cultivated both in liquid media and on plates, even though the pili could not always be detected by using transmission electron microscopy, suggesting why pili have not been found previously.

### Example 4

### The rlrA Islet is Important for Pneumococcal Adherence to Lung Epithelial Cells

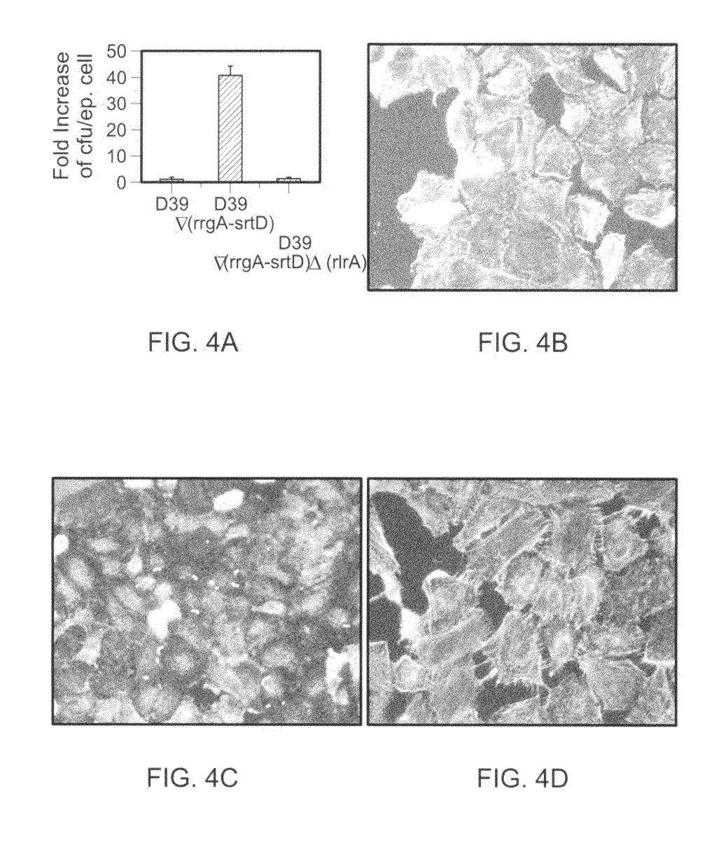

The serotype 2 strain D39, like its nonencapsulated derivative R6, lacks the rlrA islet (FIG. 2). The complete rlrA islet from T4 was introduced into D39 (D39∇(rrgA-srtD)). This islet insertion mutant of D39 expressed pili as evidenced by a ladder of HMW polymers based on Western blotting with anti-RrgB (FIG. 3B). Pilus expression in D39∇(rrgA-srtD) was dependent on the positive regulator rlrA, because no HMW polymers were detected in an rlrA mutant derivative of D39∇(rrgA-srtD) (FIG. 3B). D39, D39∇(rrgA-srtD), and D39∇(rrgA-srtD)Δ(rlrA) were used to study adherence to A549 lung epithelial cells (FIG. 4). Only pilus-expressing D39∇(rrgA-srtD) bound to these cells (FIG. 4). This binding was similar to that of pilus-expressing T4, whereas an rlrA mutant of T4 showed no detectable binding to A549 cells.

### Example 5

### The rlrA Islet Affects Virulence in Mouse Models

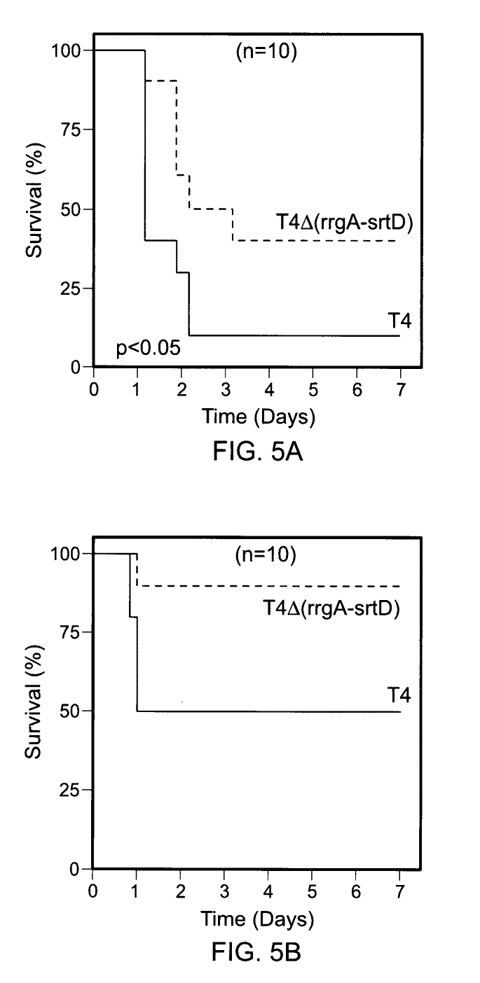

To investigate the role of the pilus in pneumococcal colonization and in invasive disease, strains T4 and T4Δ(rrgA-srtD) were used in murine infection models. To mimic the natural route of infection, 6- to 8-week-old C57BL/6 mice were inoculated intranasally with high [5×10⁶ colony-forming units (cfu)], and medium (5×10⁵ cfu) doses of pneumococci. Colonization was estimated by performing nasopharyngeal-tracheal lavages in animals postmortem. The nonpiliated mutant was less virulent than the wild-type strain as revealed by a higher survival rate of mice infected by the mutant (FIGS. 5A and 5B). This defect in virulence could be restored by reintroducing the rlrA islet.

Both wild-type and mutant pneumococci administered separately were able to colonize mice to a similar degree (not significant by Mann-Whitney U test, P>0.05). However, when equal numbers of wild-type and mutant T4 bacteria were given together intranasally, the pilus-deficient mutant was out-competed by the wild type in the upper airways, lungs, and blood, in the majority of cases (FIG. 5C-E). The type 2 strain D39, the islet insertion derivative D39∇(rrgA-srtD), and the rlrA mutant D39∇(rrgA-srtD)Δ(rlrA), were also used in competition experiments for nasopharyngeal carriage and pneumonia. The nonpiliated wild-type D39 was out-competed by the piliated islet insertion mutant D39∇(rrgA-srtD), whereas the mutant lacking rlrA was not (FIG. 5F). The present data demonstrate that pneumococcal pili play a role in colonization, pneumonia, and invasive disease.

### Example 6

### The rlrA Islet Plays a Role in Host Inflammatory Responses

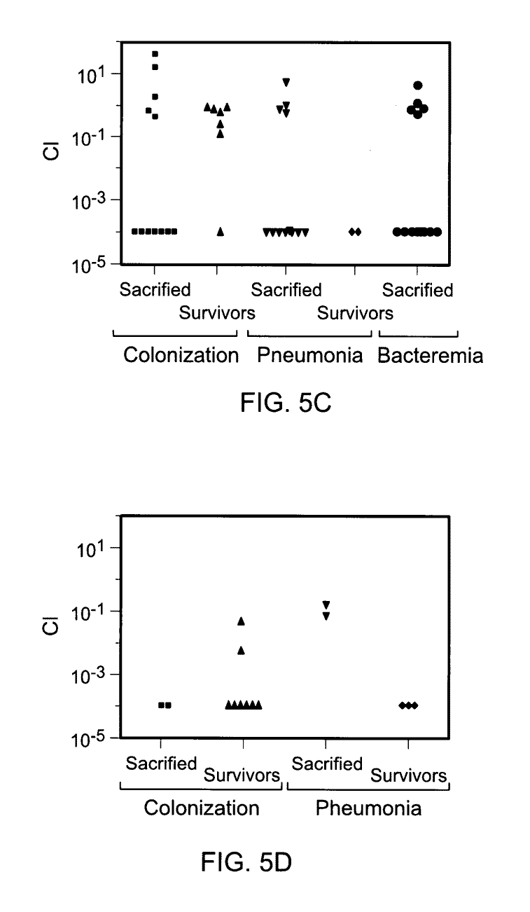

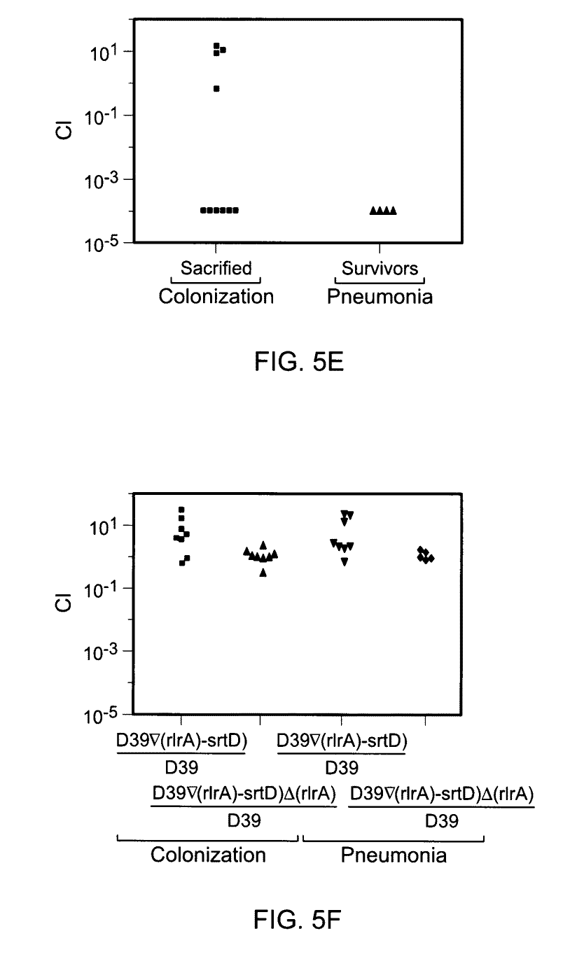

The outcome of a pneumococcal infection is affected by the host inflammatory response, which can promote bacterial clearance as well as contribute to local damage (pneumonia) or systemic damage (of which the most severe form is septic shock). We have recently shown that diverse pneumococcal clones evoke distinct proinflammatory cytokine responses when given i.p. to mice (26). A serotype 6B strain and the T4 and ST1621^{9F} strains, shown here to produce pili, all evoked a high TNF response after i.p. challenge (5). In contrast, a serotype 19F strain of a different clonal type, ST425^{19F}, was not as efficient in colonizing the upper airways of mice and evoked a low TNF response (5). This was also true for a serotype 1 and a serotype 7F isolate (5, 22), which belong to invasive clonal types associated with relatively mild invasive disease and no mortality in humans (22). These clones were analyzed for the presence of the rlrA pilus islet by PCR, sequencing, and Southern blot hybridization. Results demonstrated that rlrA islet-positive pneumococcal strains (ST205⁴ and ST162^{19F} of type 4 and 19F, respectively) elicited a high cytokine response, whereas rlrA islet-negative strains (ST191^{7F}, ST228′, and ST306¹ of type 7F and 1, respectively) induced a low TNF response (5). Presence or absence of the pneumococcal pilus islet could therefore explain the difference in TNF response. To test this possibility directly, the inflammatory response was measured during invasive pneumococcal infection after challenging mice i.p. with piliated wild-type and rrgA-srtD deletion mutants lacking pili. Infections with the two deletion mutants were also performed with higher infection doses to ensure that the low TNF responses were not due to lower numbers of bacteria in the blood stream. The pilus deletion mutants in T4 as well as ST162^{19F} backgrounds showed a significantly lower TNF response (FIG. 6) and IL-6 response (FIG. 7) compared with their respective wild-type strains. By plotting TNF values against bacterial numbers it was evident that the TNF response to piliated pneumococci was significantly higher than to the equivalent number of nonpiliated pneumococci (FIGS. 6C and D). Furthermore, reintroduction of the rlrA islet into T4Δ(rrgA-srtD) restored the high TNF response of piliated T4.

These results demonstrate that S. pneumoniae produces pilus-like structures that project from the bacterial cell surface. The pneumococcal pilus is encoded by the rlrA pilus islet, found in some but not all pneumococcal strains. In encapsulated S. pneumoniae, pili contribute to adhesion to cultured epithelial cells, and to colonization and invasive disease in murine models of infection. Pili expression also augments the host inflammatory response. Pneumococci use a variety of mechanisms to interact with their host at different stages of infection. Expression of pili can facilitate the initial bacterial adherence, promoting colonization of the nasopharynx. Simultaneously, bacteria expressing these structures can be more prone to trigger mucosal inflammation that can promote clearance, but potentially also can lead to invasion of pneumococci into the tissue, if inflammation leads to damage of the mucosal barrier.

### Example 7

### Purification of Streptococcus pneumoniae pili

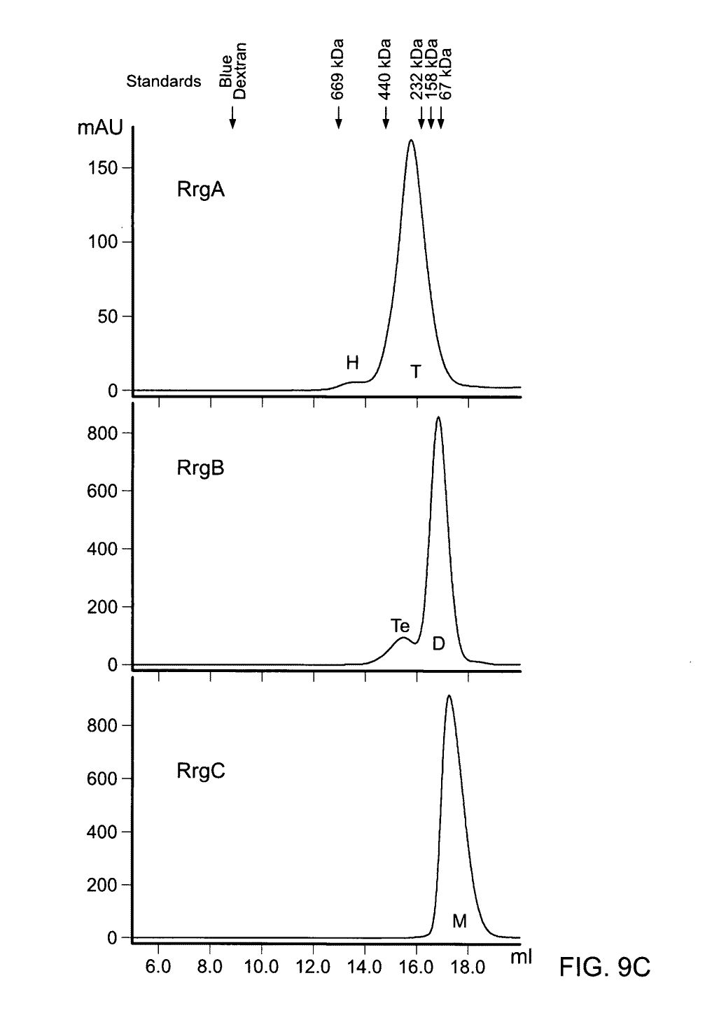

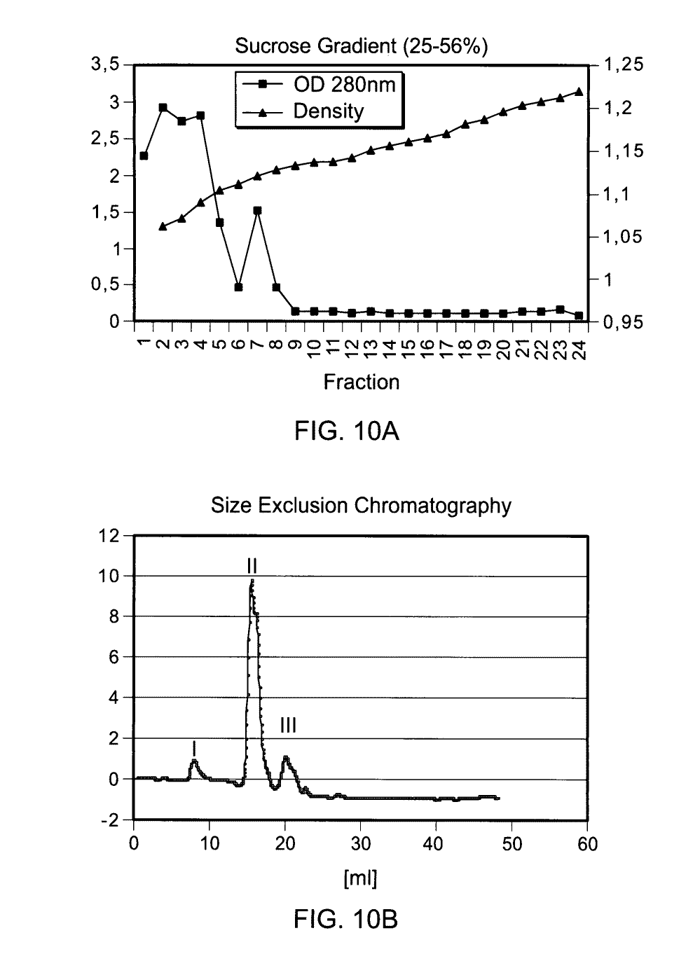

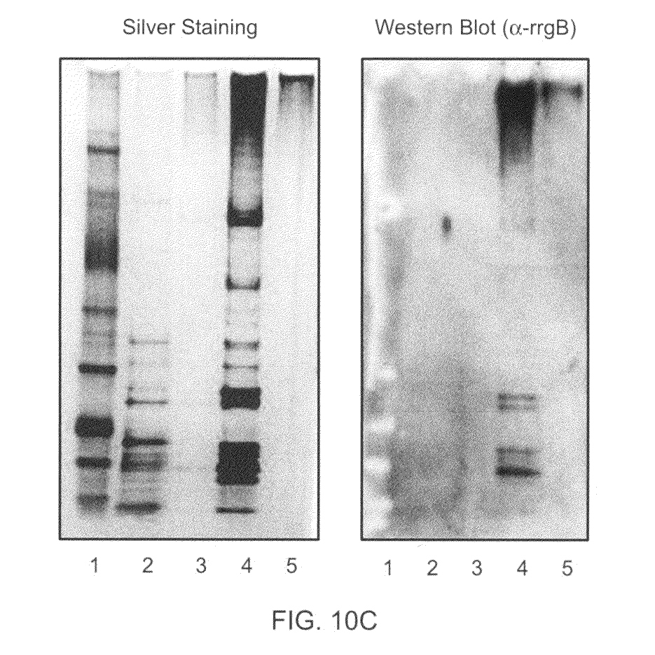

S. pneumoniae TIGR4 glycerol stock (−80° C.) was grown on tryptic soy agar supplemented with 5% defibrinated mutton blood (overnight at 37° C. in 5% CO₂). Fresh bacteria were used to incubate new agar plates and cultivated for about 12 hours at 37° C. in 5% CO₂. Harvested bacteria of about 10 plates were washed once in 35 ml PBS, and resuspended in 2 ml protoplast buffer PPB (10 mM MgCl₂, 50 mM sodium phosphate pH 6.3, 20% sucrose) containing protease inhibitor cocktail set (Calbiochem). About 450 U of mutanolysin in 100 mM sodium phosphate pH 6.3 were added to each half of the suspension and incubated at 37° C. for about 5 to 8 hours with gentle shaking until protoplast formation was detected by microscopy. Supernatant containing digested pilus material was loaded on a sucrose gradient (25 to 56% in 10 mM MgCl₂, 50 mM sodium phosphate pH 6.3) and run for about 20 hours at 38,000 rpm at 4° C. (FIG. 10A). All further steps were performed at 4° C. using buffers containing protease inhibitors. In addition, Benzonase™ nuclease (Novagen) was added to remove DNA and RNA impurities. Collected gradient fractions were tested for pilus material using anti-RrgB antibodies. Pilus-containing fractions were pooled and dialyzed against 10 mM MgCl₂, 50 mM sodium phosphate pH 6.3 for about one day to remove sucrose.

To reduce polydispersity, additional chromatography steps were added. When necessary, pooled pilus preparations were concentrated before loading them on a Superose™ 6 10/300 GL column (Amersham Biosciences) (FIG. 10B). Gel filtration resulted in separation of pilus containing material of different molecular weights. Purified pilus fractions were judged to be homogeneous based on electron microscopy and sodium dodecyl sulphate polyacrylamide gel electrophoresis and immunoblotting with an antibody specific form RrgB (FIG. 10C). Samples were stored at −80° C. or in liquid nitrogen until further use.

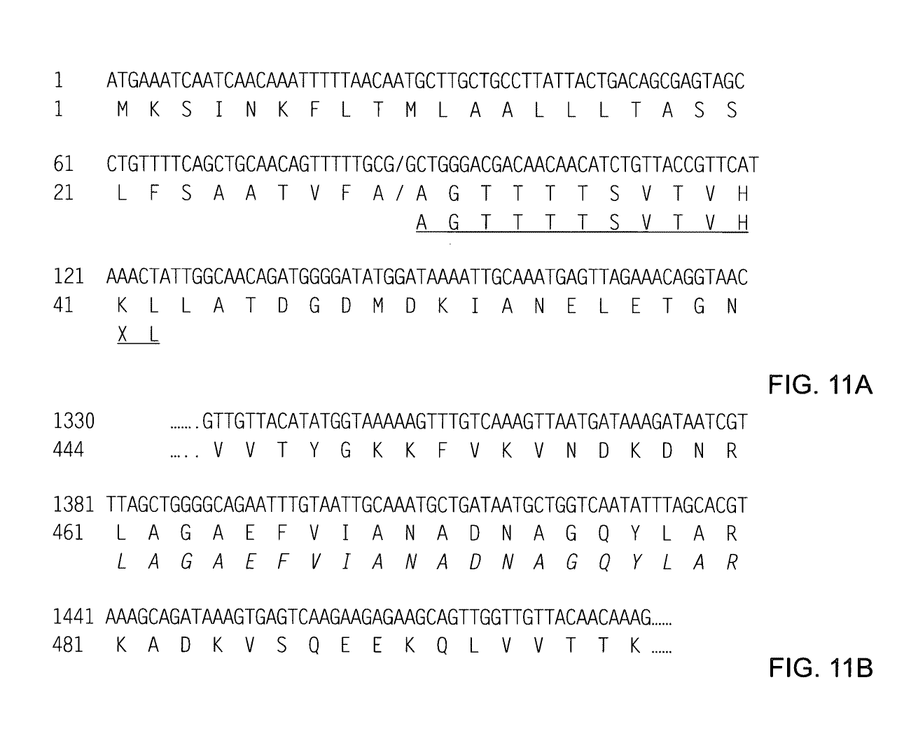

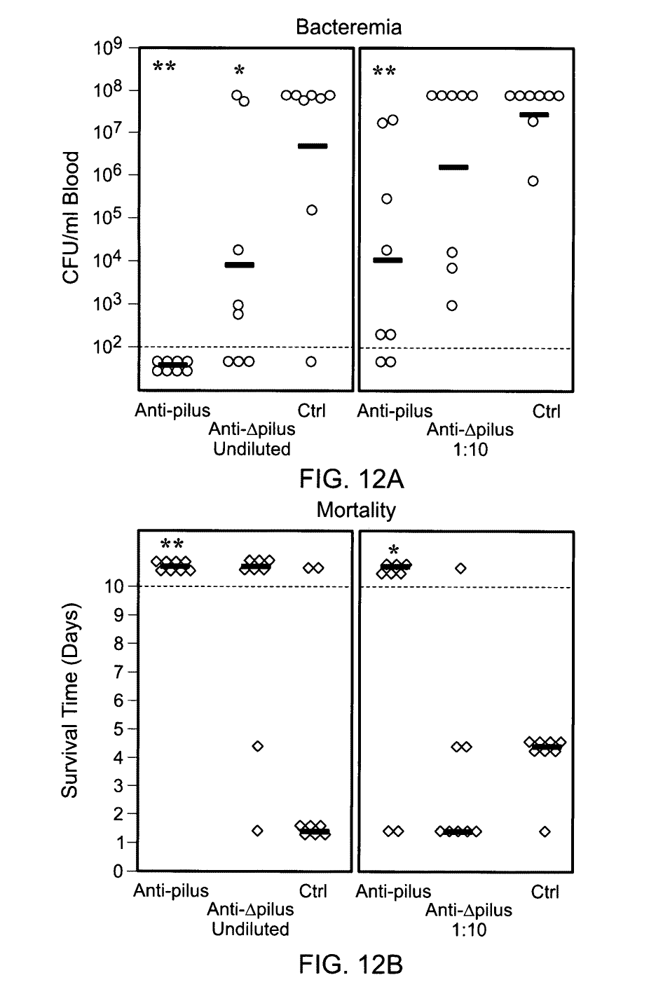

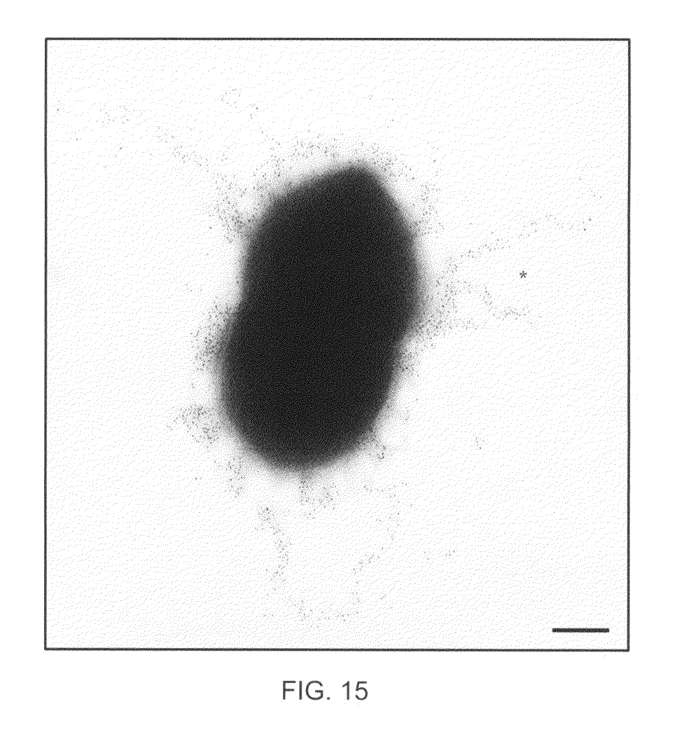

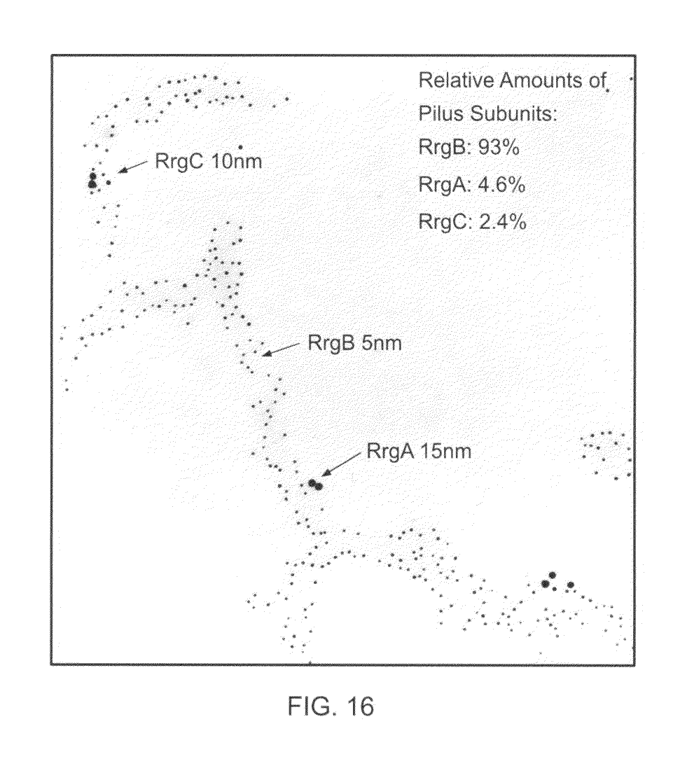

High molecular weight purified pili showed molecular masses ranging from 2×10⁶ to 3×10⁶ Da. Heat treatment of pili in the presence of SDS resulted in its dissociation into smaller molecules, yielding a ladder of lower-molecular-weight bands on a polyacrylamide-SDS gel. Edmann degradation of purified pili identified a sequence that corresponds to the predicted N-terminus of the RrgB protein produced by cleavage of the signal sequence (AGTTTTSVTVHXL; SEQ ID NO:56) (FIG. 11A). Amino acid sequence analysis of pilus tryptic peptide sequences identified a fragment of pneumococcus TIGR4 RrgB protein with the amino acid sequence LAGAEFVIANADNAGQYLAR (SEQ ID NO:7) (FIG. 11B). Electron microscopy investigation was performed on negative stained (1% PTA), immunogold labelled purified pili preparations. Elongated tubular filaments up to 1 μm long and about 10 nm in diameter were observed, similar to those detected on whole bacteria. Besides single pili filaments, bundles of strictly packed individual structures were observed. Antiserum against purified RrgB and RrgC reacted with isolated pili under immunogold EM (FIG. 15) and in western analysis (FIG. 10C). The gold labeling pattern of anti-RrgA, anti-RrgB, and anti-RrgC is shown in FIG. 16.

### Example 8

### Pilus Proteins RrgA and RrgB Associate In Vitro

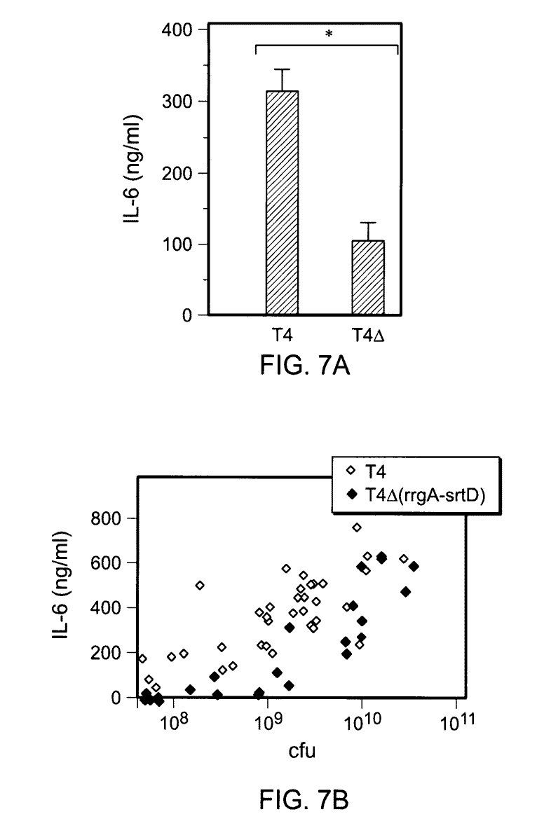

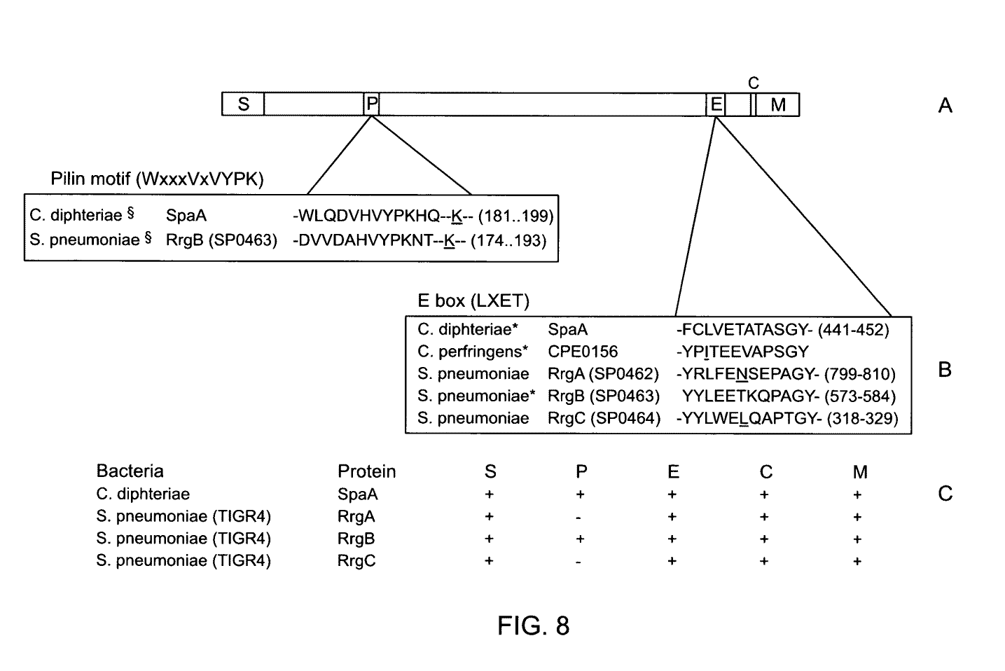

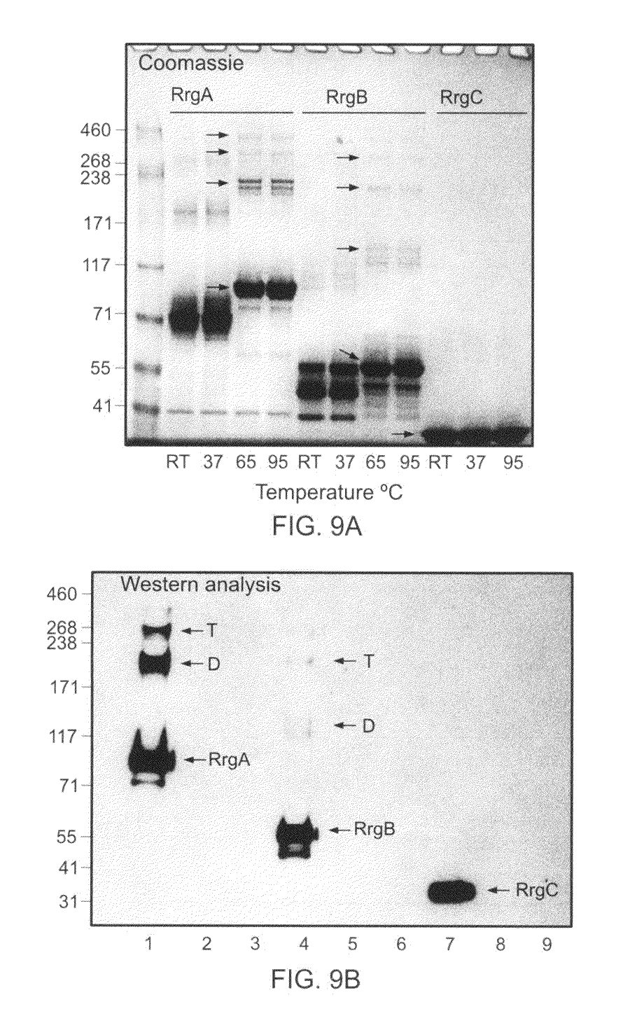

Pilus proteins RrgA, RrgB and RrgC were purified as described in Example 1. The purified protein preparations were incubated in vitro at room temperature, 37° C., 65° C., and 95° C. for 5 minutes. The incubated preparations were run on a denaturing polyacrylamide gel. High molecular weight complexes were observed in the RrgA and RrgB preparations, but not in the RrgC-His preparations (FIG. 9A). The presence of RrgA and RrgB in the high molecular weight complexes was confirmed by Western blotting (FIG. 9B). High molecular weight complexes were also detected in the RrgA and RrgB preparations by size exclusion chromatography (FIG. 9C).

### Example 9

### Antisera Prepared Against Pili are Protective Against Infection

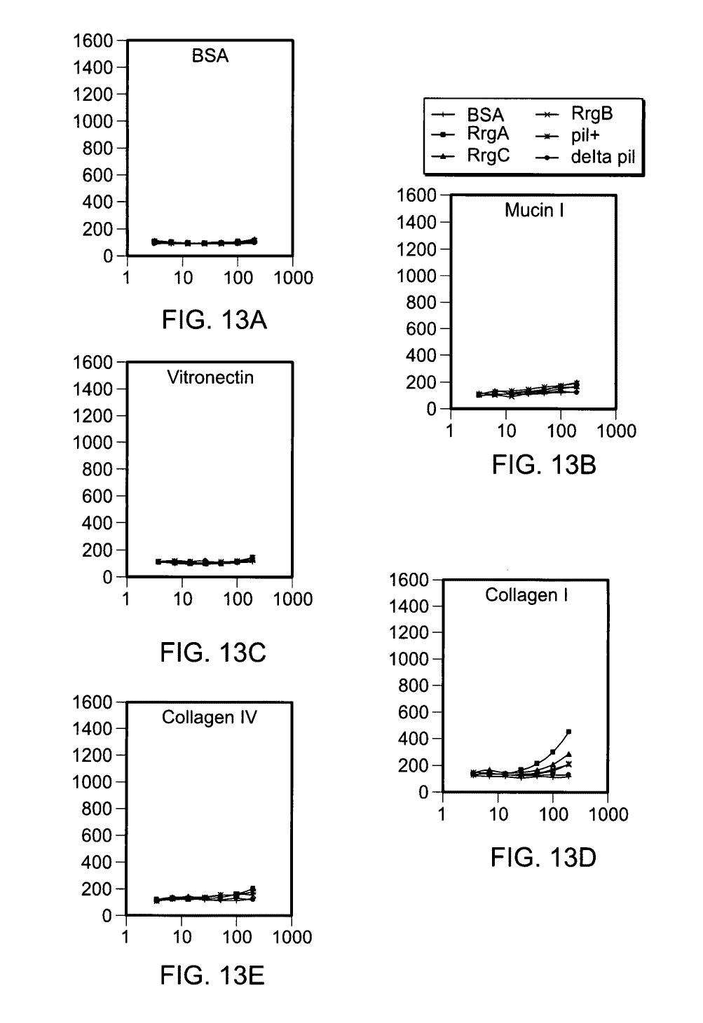

Mice were challenged i.p. with T4 bacteria as described in Example 1, except the mice were administered antisera against purified pili (anti-pilus), antisera against a preparation purified under identical conditions from bacteria that do not produce pili (anti-Apilus), or saline control (ctrl). In parallel experiments, the mice were administered identical antisera diluted 1:10. Animals were observed over ten days for mortality, and bacterial load was measured at 24 hours post challenge. All of the mice treated with undiluted anti-pilus sera had bacterial loads below the level of detection; treatment with 1:10 diluted sera still provided some protection (FIG. 12A). Both diluted and undiluted anti-pilus sera provided a significant reduction of mortality compared to the saline control (FIG. 12B). Furthermore, the sera prepared against pili provided greater protection against bacteremia and mortality than the anti-Apilus sera (FIGS. 12A-B). This example demonstrates that sera specific for purified pili provided significant protection against S. pneumoniae infection in an animal model.

### Example 10

### Purified Pili and Pilus Proteins Bind to Extracellular Matrix Components

Binding of RrgA, RrgB, RrgC, purified pili, and mock-purified pili to extracellular components was determined by ELISA. Binding of pili components to extracellular matrix components mucin I, vitronectin, lactoferrin, collagens I and IV, laminin, Fibronectin and Fibrinogen was measured. Briefly, Maxisorp™ 96-well flat-bottom plates (Nunc, Roskilde, Denmark) were coated for 1 hour at 37° C. followed by an overnight incubation at 4° C. with 2 μg/well with mucin I, vitronectin, lactoferrin, collagens I and IV and Fibrinogen and with 1 μg/well with laminin and fibronectin in phosphate-buffered saline pH 7.4 (PBS). A BSA coated plate served as a negative control. The coated wells were washed 3 times with PBS/0.05% Tween™ 20 and blocked for 2 hours at 37° C. with 200 μl of 1% BSA. The plates were washed 3 times with PBS/0.05% Tween™ 20. Protein samples (RrgA, RrgB and RrgC) were initially diluted to 0.4 μg/μl. with PBS. 200 μl of protein solution and 25 μl pilus preparation (in 200 μl total volume with PBS) and respective controls were transferred into coated-blocked plates in which the samples were serially diluted two-fold with PBS. Plates were incubated for 2 hours at 37° C. and overnight at 4° C. The plates were washed 3 times with PBS/0.05% Tween™ 20 and incubated for 2 hours at 37° C. with primary mouse anti-Rrg antibodies ( 1/10,000 dilutions): RrgA, RrgB and RrgC coated plates with anti-RrgA, anti-RrgB and anti-RrgC respectively, pilus coated plates were incubated with anti-RrgB antibodies. After another 3 washing steps, antigen-specific IgG was revealed with alkaline phosphatase-conjugated goat anti-mouse IgG (Sigma Chemical Co., SA Louis, Mo.) after 2 hours of incubation at 37° C.

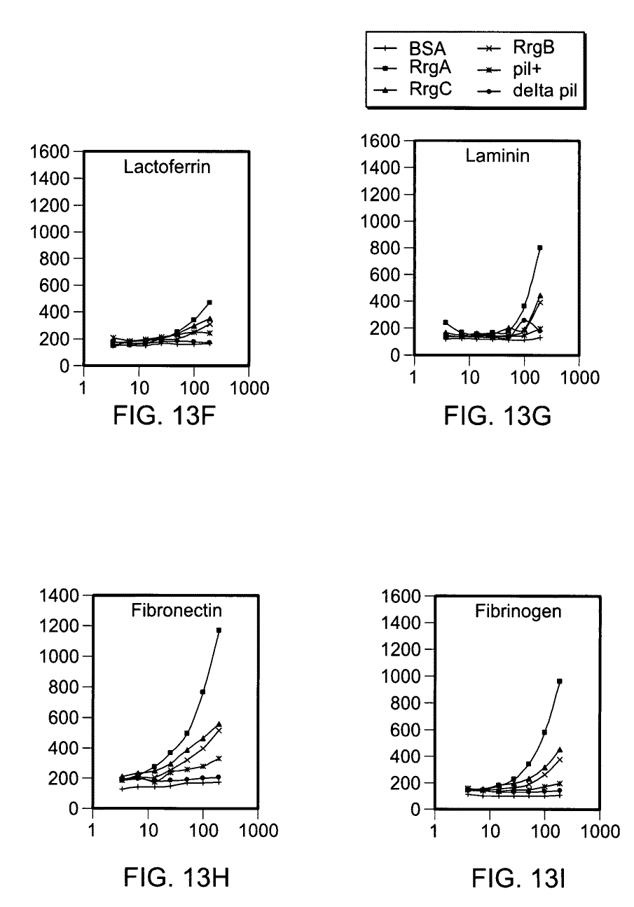

Significant binding was observed to collagen I, lactoferrin, laminin, fibronectin, and fibrinogen (FIG. 13). In all cases, the strongest binding was observed for RrgA followed by RrgC and RrgB at lower levels. Purified pili showed lesser but detectable binding. This example demonstrates binding of purified pili and isolated pilus proteins to extracellular matrix components and suggests a function of pili in adhesion/colonization.

### Example 11

### Purified Pili Induce Cytokine Responses In Vitro

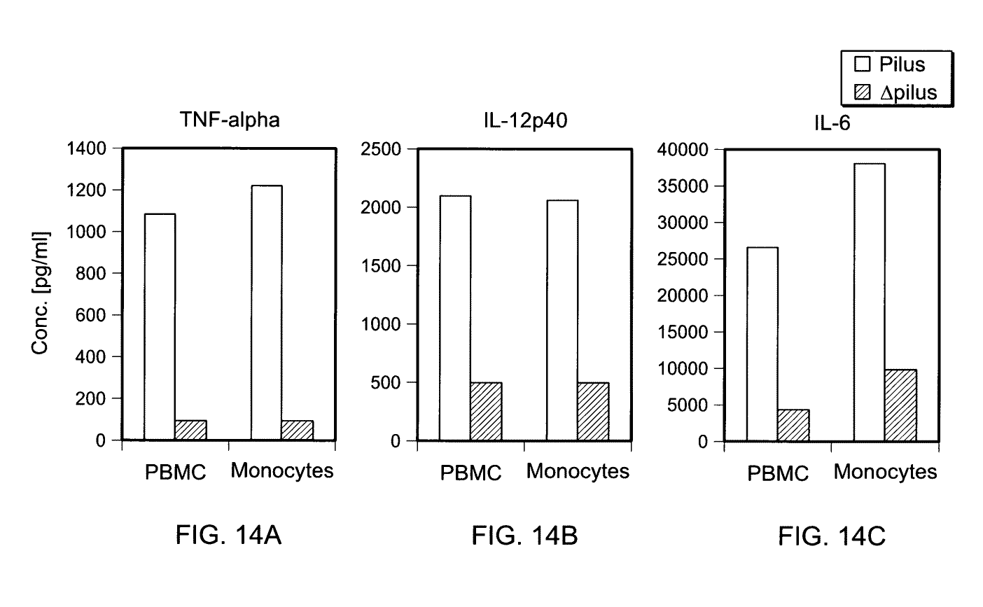

Peripheral blood mononuclear cells (PBMCs) and monocytes were contacted in vitro with a purified pilus preparation and a mock preparation purified from T4 that do not express pili. Production of cytokines by the cells in response to pili was measured by ELISA. Purified pili induced production of inflammatory cytokines TNF-alpha, IL-12p40, and IL-6 compared to the delta pilus control (FIG. 14). No induction was observed for TLRs 2, 7, 8 and 9.

### Example 12

### Electron Microscopy Analysis of Purified Pili

Five microliters of the purified pili preparation were placed on a 300-mesh copper grid coated with a thin carbon film. The grids were then negatively stained by adding 5 microliters of 1% PTA (phosphotungstic acid). The excess liquid was blotted.

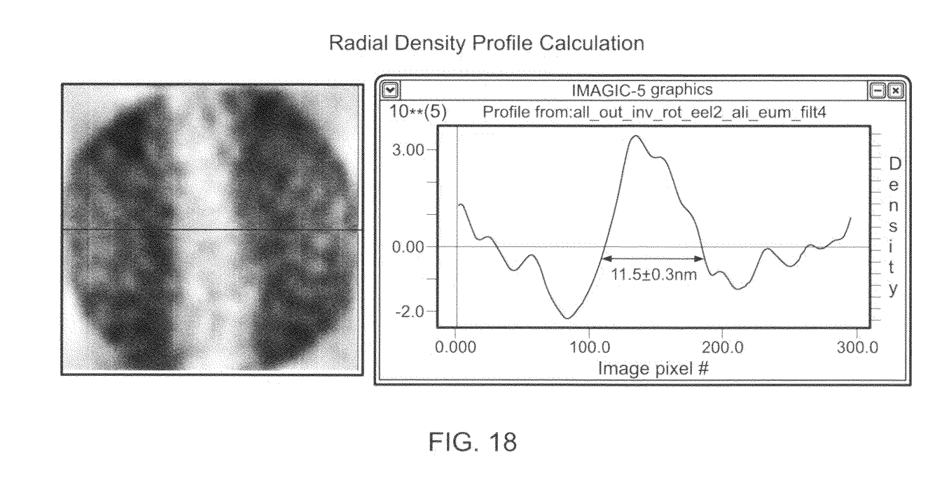

The grids were observed using a FEG200 electron microscope. The images were recorded at an accelerating voltage of 100 kV and nominal magnification of 50000× under low-dose conditions. Pili were observed as elongated, flexible structures (FIG. 18).

The electron micrographs were scanned by and the images were than converted to IMAGIC 5 format (imagic5.de). Identical portions of pili were picked from the digitized negatives by using squared boxes (300×300 pixels) by using EMAN software. Between the whole boxed pili collection only straight pili with same growth direction and similar diameter were processed.

In first analysis, the boxed pili were inverted in density, high-pass and low-pass filtered and than aligned to the projection of a model cylinder with the same diameter (FIG. 18). Rotational alignment was applied using self-correlation function followed by translational alignment perpendicular to the cylinder axis only.

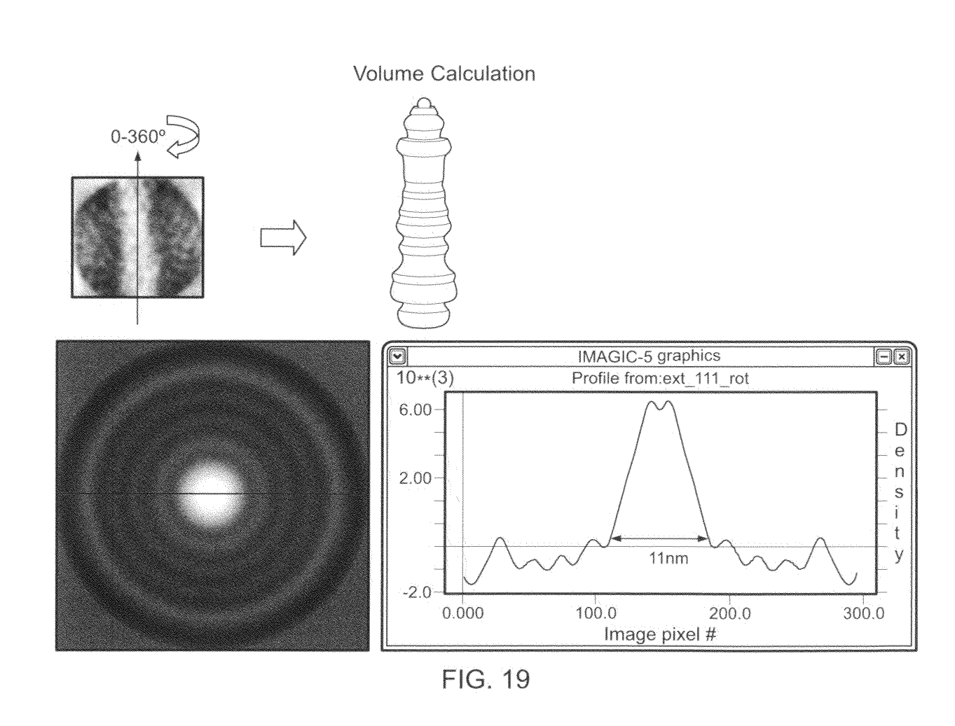

Density profile across the filament axis of the average was calculated and showed by graphical representation. The density profile strongly indicated that the pilus is a compact, solid structure with no hole in the middle and that the overall structure has a calculated average diameter of 11.5 nm (FIG. 18). A similar diameter (11 nm) was calculated from the rotationally symmetrized three-dimensional volume obtained by assigning angles of rotation randomly to the aligned stalk segments (FIG. 19). Moreover, the volume showed that the pili surface is not smooth (FIGS. 18-19).

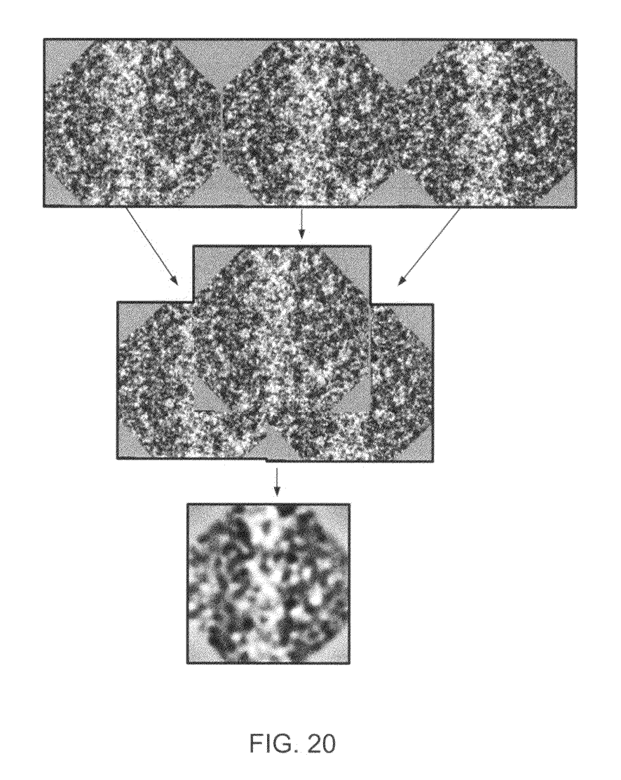

Several of the pre-aligned stalk segments presenting strong structural features of a 13 nm repeat have been further aligned by axial averaging generating an improved 2D image with a stronger signal (FIG. 20).

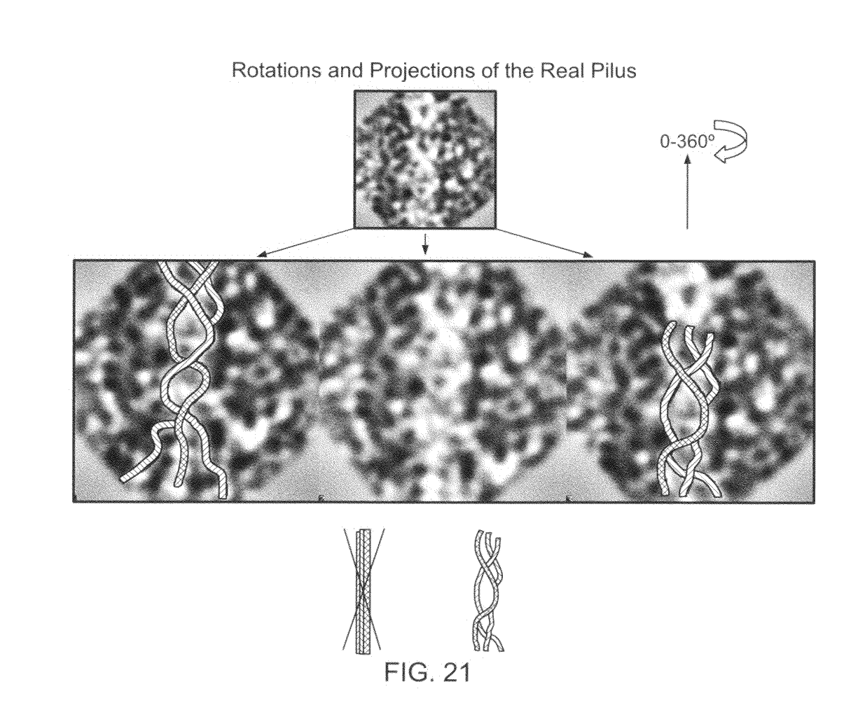

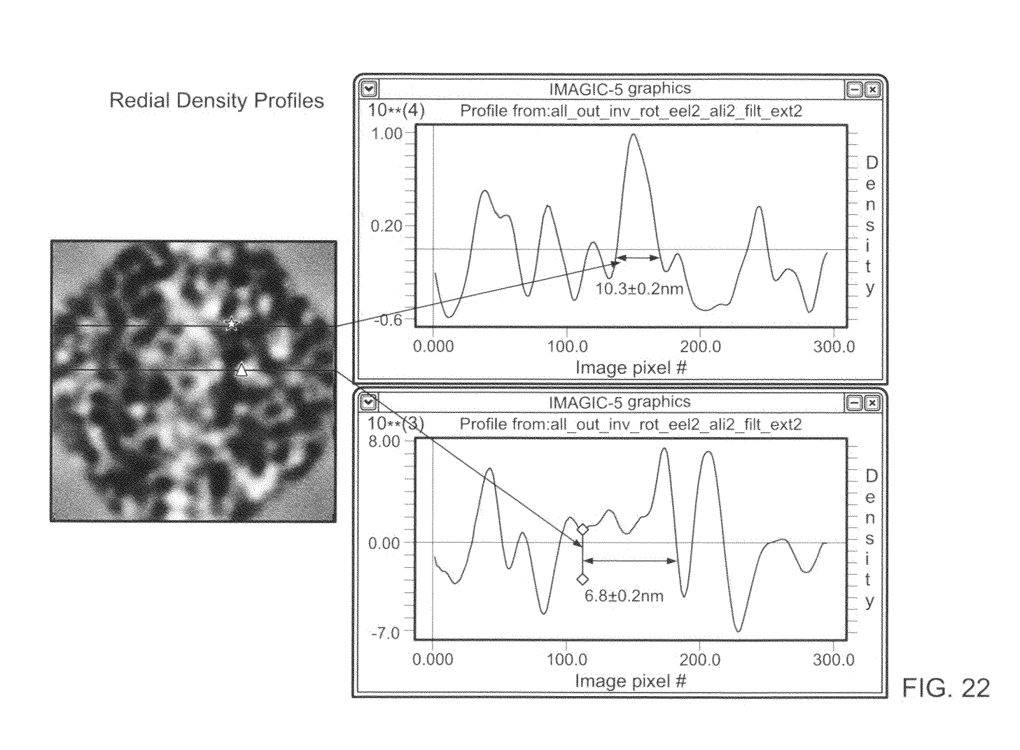

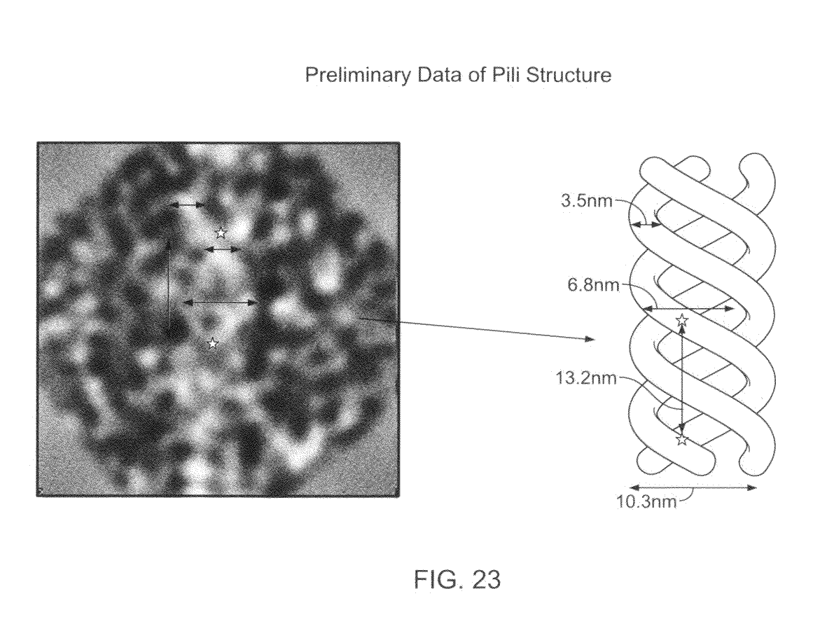

The 2D images (projections of a 3D structure) (FIGS. 21-22) show clearly that the pili are made by at least 3 “protofilaments” arranged in a coiled-coil structure with an average diameter of 10.5-11.0 nm and a pich of 13.2 nm (FIG. 23). The diameter of the pili at the node position is 6.8 nm and every single “protofilament” has a diameter of 3.5 nm (FIG. 23).

### REFERENCES (EACH OF WHICH IS INCORPORATED BY REFERENCE IN ITS ENTIRETY)

1. Bruyn, G. A. W. & van Furth, R. (1991) Eur. J. Clin. Microbiol. Infect. Dis. 10, 897-910.
2. Ryan, M. W. & Antonelli, P. J. (2000) Laryngoscope 110, 961-964.
3. Cutts, F. T., Zaman, S. M., Enwere, G., Jaffar, S., Levine, O. S., Okoko, C. Oluwalana, A., Vaughan, S., Obaro, A., Leach, A., et al. (2005) Lancet 365, 1139-1146.
4. Swiatlo, E., Champlin, F. R., Holman, S. C., Wilson, W. W.&Watt, J. M. (2002) Infect. Immun. 70, 412-415.
5. Sandgren, A., Albiger, B., Orihuela, C., Tuomanen, E., Normark, S. & Henriques-Normark, B. (2005) J. Infect. Dis. 192, 791-800.
6. Henriques Normark, B., Christensson, B., Sandgren, A., Noreen, B., Sylvan, S., Burman, L. G. & Olsson-Liljequist, B. (2003) Microb. Drug Resist. 9, 337-344.
7. Nunes, S., Sá-Leão, R., Carriço, J., Alves, C. R., Mato, R., Avô, A. B., Saldanha, J., Almeida, J. S., Sanches, I. S. & de Lencastre, H. (2005) J. Clin. Microbiol. 43, 1285-1293.
8. Henrichsen, J. (1995) J. Clin. Microbiol. 33, 2759-2762.
9. Lau, G. W., Haataja, S., Lonetto, M., Kensit, S. E., Marra, A., Bryant, A. P., McDevitt, D., Morrison, D. A. & Holden, D. W. (2001) Mol. Microbiol. 40, 555-571.
10. Rosenow, C., Ryan, P., Weiser, J. N., Johnson, S., Fontan, P., Ortqvist, A. & Masure, H. R. (1997) Mol. Microbiol. 25, 819-829.
11. Tuomanen, E. (1999) Current Opin. Biol. 2, 35-39.
12. Ton-That, H., Marraffini, L. A. & Schneewind, O. (2004) Mol. Microbiol. 53, 251-261.
13. Ton-That, H. & Schneewind, O. (2003) Mol. Microbiol. 50, 1429-1438.
14. Kelstrup, J., Theilade, J. & Fejerskov, O. (1979) Scand. J. Dent. Res. 87, 415-423.
15. Mora, M., Bensi, G., Capo, S., Falugi, F., Zingaretti, C., Manetti, A. G. O., Maggi, T., Taddei, A. R., Grandi, G. & Telford, J. L. (2005) Proc. Natl. Acad. Sci. USA 102, 15641-15646.
16. Lauer, P., Rinaudo, C. D., Soriani, M., Margarit, I., Mainone, D., Rosini, R., Taddei, A. R., Mora, M., Rappuoli, R., Grandi, G. & Telford, J. L. (2005) Science 309, 105.
17. Li, T., Khah, M. K., Slavnic, S., Johansson, I. & Strömberg, N. (2001) Infect Immun. 69, 7224-7233.
18. Hava, D. L., Hemsley, C. J. & Camilli, A. (2003) J. Bacteriol. 185, 413-421.
19. Hava, D. L. & Camilli, A. (2002) Mol. Microbiol. 45, 1389-1406.
20. Schwarz-Linek, U., Hook, M. & Potts, J. R. (2004) Mol. Microbiol. 52, 631-641.
21. Hemsley, C., Joyce, E., Hava, D. L., Kawale, A.&Camilli, A. (2003) J. Bacteriol. 185, 6640-6647.
22. Sjöstrom, K., Spindler, K., Örtqvist, A., Kalin, M., Sandgren, A. & Henriques Normark, B. (2006) Clin. Infect. Dis., in press.
23. Lau, P. C. Y., Sung, C. K., Lee, J. H., Morrison, D. A. & Cvitkovitch, G. D. (2002) J. Microbiol. Methods 49, 193-205.
24. Sung, C. K., Li, H., Claverys, J. P. & Morrison, D. A. (2001) Appl. Environ. Microbiol. 67, 5190-5196.
25. Bricker, A. L. & Camilli, A. (1999) FEMS Microbiol. Lett. 172, 131-135.
26. Albiger, B., Sandgren, A., Katsuragi, H., Meyer-Hoffert, U., Beiter, K., Wartha, F., Hornef, M., Normark, S. & Henriques Normark, B. (2005) Cell. Microbiol. 7, 1603-1615.
27. Fernebro, J., Andersson, I., Sublett, J., Morfeldt, E., Novak, R., Tuomanen, E., Normark, S. and Henriques Normark, B. (2004) J. Infect. Dis. 189, 328-338.
28. Gosink, K. K., Mann, E. R., Guglielmo, C., Tuomanen, E. I. & Masure, H. R. (2000) Infect. Immun. 68, 5690-5695.
29. Iannelli, F., Pearce, B. J. & Pozzi, G. (1999) J. Bacteriol. 181, 2652-2654.

### OTHER EMBODIMENTS

A number of embodiments of the invention have been described. Nevertheless, it will be understood that various modifications may be made without departing from the spirit and scope of the invention.

1. A method of producing an isolated Streptococcus pneumoniae pilus comprising subjecting a bacterial cell that produces the pilus to mechanical shearing which comprises ultrasonication and isolating the pilus from the cell.

2. A method of isolating Streptococcus pneumoniae pili comprising:
subjecting bacterial cells that produce Streptococcus pneumoniae pili to mechanical shearing which comprises ultrasonication; and
isolating the pili from the cells.

3. The method of claim 2, wherein isolating comprises one or more density gradient centrifugations or chromatography steps.

4. The method of claim 2, wherein the step of isolating comprises reducing polydispersity.

5. A method of isolating Streptococcus pneumoniae pili, the method comprising:
subjecting Streptococcus pneumoniae cells that produce Streptococcus pneumoniae pili to ultrasonication or digestion with a lytic enzyme;
separating non-cellular components;
degrading nucleic acids with a nuclease; and
isolating Streptococcus pneumoniae pili.

6. The method of claim 5, wherein the Streptococcus pneumoniae cells that produce Streptococcus pneumoniae pili are subjected to digestion with the lytic enzyme and the lytic enzyme is mutanolysin.

7. The method of claim 5 wherein non-cellular components are separated using density gradient centrifugation.

8. The method of claim 5, wherein the Streptococcus pneumoniae cells that produce Streptococcus pneumoniae pili are Streptococcus pneumoniae serotype 4 strain T4 (TIGR4) cells.

9. The method of claim 5, wherein the method further comprises reducing polydispersity by separating the Streptococcus pneumoniae pili by size using gel filtration chromatography.

10. The method of claim 5 wherein the Streptococcus pneumoniae cells that produce Streptococcus pneumoniae pili are subjected to ultrasonication.

11. An isolated pilus or pilus-like multimer comprising a polypeptide comprising the amino acid sequence of a Streptococcus pneumoniae pilus protein with up to 30 amino acid substitutions, insertions, or deletions.

12. The pilus or pilus-like multimer of claim 11 with up to 20 amino acid substitutions, insertions, or deletions.

13. The pilus or pilus-like multimer of claim 11 with up to 10 amino acid substitutions, insertions, or deletions.

14. The pilus or pilus-like multimer of claim 11 with up to 5 amino acid substitutions, insertions, or deletions.
